SEQUENCE LISTING (351 sequences)

SEQ ID NO: 1 - protein, 6 aa, Artificial Sequence
  anti-TLR4 variable heavy chain complementarity determining region 1
GGYSWH

SEQ ID NO: 2 - protein, 16 aa, Artificial Sequence
  anti-TLR4 variable heavy chain complementarity determining region 2
YIHYSGYTDFNPSLKT

SEQ ID NO: 3 - protein, 9 aa, Artificial Sequence
  anti-TLR4 variable heavy chain complementarity determining region 3
KDPSDAFPY

SEQ ID NO: 4 - protein, 11 aa, Artificial Sequence
  anti-TLR4 variable light chain complementarity determining region 1
RASQSISDHLH

SEQ ID NO: 5 - protein, 7 aa, Artificial Sequence
  anti-TLR4 variable light chain complementarity determining region 2
YASHAIS

SEQ ID NO: 6 - protein, 9 aa, Artificial Sequence
  anti-TLR4 variable light chain complementarity determining region 3
QQGHSFPLT

SEQ ID NO: 7 - protein, 118 aa, Artificial Sequence
  anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDPSDAFPYWGQGTLVTVSS

SEQ ID NO: 8 - protein, 107 aa, Artificial Sequence
  anti-TLR4 variable light chain
EIVLTQSPDFQSVTPKEKVTITCRASQSISDHLHWYQQKPDQSPKLLIKYASHAISGVPSRFSGSGSGTDFTLTINSLEAEDAATYYCQQGHSFPLTFGGGTKVEIK

SEQ ID NO: 9 - protein, 467 aa, Artificial Sequence
  anti-TLR4 heavy chain
MGWSWIFLFLLSGTAGVHCQVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDPSDAFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 233 aa, Artificial Sequence
  anti-TLR4 light chain
MEWSWVFLFFLSVTTGVHSEIVLTQSPDFQSVTPKEKVTITCRASQSISDHLHWYQQKPDQSPKLLIKYASHAISGVPSRFSGSGSGTDFTLTINSLEAEDAATYYCQQGHSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 12 - DNA, 1404 bp, Artificial Sequence
  NI-0101 heavy chain
atgggatggagctggatctttctcttcctcctgtcaggaactgcaggtgtacattgccaggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgacgcctttccttactggggccaagggactctggtcactgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaaatgcaaggtctccagtaaagctttccctgcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 13 - DNA, 702 bp, Artificial Sequence
  NI-0101 light chain
atggaatggagctgggtctttctcttcttcctgtcagtaactacaggtgtccactccgaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggtcacagttttccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - protein, 118 aa, Artificial Sequence
  15C1 Hu VH version 4-28
  misc_feature (30)..(30): Xaa may be Thr or Ser
  misc_feature (49)..(49): Xaa may be Ile or Met
  misc_feature (68)..(68): Xaa may be Val or Ile
  misc_feature (70)..(70): Xaa may be Met or Ile
QVQLQESGPGLVKPSDTLSLTCAVSGYSIXGGYSWHWIRQPPGKGLEWXGYIHYSGYTDFNPSLKTRXTXSRDTSKNQFSLKLSSVTAVDTAVYYCARKDPSDGFPYWGQGTLVTVSS

SEQ ID NO: 15 - protein, 118 aa, Artificial Sequence
  15C1 Hu VH version 3-66
  misc_feature (24)..(24): Xaa may be Ala or Val
  misc_feature (49)..(49): Xaa may be Val or Met
  misc_feature (79)..(79): Xaa may be Leu or Phe
EVQLVESGGGLVQPGGSLRLSCAXSGYSITGGYSWHWVRQAPGKGLEWXSYIHYSGYTDFNPSLKTRFTISRDNSKNTXYLQMNSLRAEDTAVYYCARKDPSDGFPYWGQGTLVTVSS

SEQ ID NO: 16 - protein, 107 aa, Artificial Sequence
  15C1 Hu VL version L6
  misc_feature (49)..(49): Xaa may be Lys or Tyr
EIVLTQSPATLSLSPGERATLSCRASQSISDHLHWYQQKPGQAPRLLIXYASHAISGIPARFSGSGSGTDFTLTISSLEPEDFAVYYCQNGHSFPLTFGGGTKVEIK

SEQ ID NO: 17 - protein, 9 aa, Artificial Sequence
  15C1 Hu VL version L6 CDR3
QNGHSFPLT

SEQ ID NO: 18 - protein, 107 aa, Artificial Sequence
  15C1 Hu VL version A26
EIVLTQSPDFQSVTPKEKVTITCRASQSISDHLHWYQQKPDQSPKLLIKYASHAISGVPSRFSGSGSGTDFTLTINSLEAEDAATYYCQNGHSFPLTFGGGTKVEIK

SEQ ID NO: 19 - protein, 120 aa, Artificial Sequence
  18H10 Hu VH version 1-69
  misc_feature (48)..(48): Xaa may be Met or Ile
  misc_feature (74)..(74): Xaa may be Lys or Thr
  misc_feature (81)..(81): Xaa may be Met or Leu
QVQLVQSGAEVKKPGSSVKVSCKASGFNIKDSYIHWVRQAPGQGLEWXGWTDPENVNSIYDPRFQGRVTITADXSTSTAYXELSSLRSEDTAVYYCARGYNGVYYAMDYWGQGTTVTVSS

SEQ ID NO: 20 - protein, 5 aa, Artificial Sequence
  18H10 Hu VH version 1-69 CDR1
DSYIH

SEQ ID NO: 21 - protein, 17 aa, Artificial Sequence
  18H10 Hu VH version 1-69 CDR2
WTDPENVNSIYDPRFQG

SEQ ID NO: 22 - protein, 11 aa, Artificial Sequence
  18H10 Hu VH version 1-69 CDR3
GYNGVYYAMDY

SEQ ID NO: 23 - protein, 106 aa, Artificial Sequence
  18H10 Hu VL version L6
  misc_feature (70)..(70): Xaa may be Phe or Tyr
EIVLTQSPATLSLSPGERATLSCSASSSVIYMHWYQQKPGQAPRLLIYRTYNLASGIPARFSGSGSGTDXTLTISSLEPEDFAVYYCHQWSSFPYTFGQGTKVEIK

SEQ ID NO: 24 - protein, 10 aa, Artificial Sequence
  18H10 Hu VL version L6 CDR1
SASSSVIYMH

SEQ ID NO: 25 - protein, 7 aa, Artificial Sequence
  18H10 Hu VL version L6 CDR2
RTYNLAS

SEQ ID NO: 26 - protein, 9 aa, Artificial Sequence
  18H10 Hu VL version L6 CDR3
HQWSSFPYT

SEQ ID NO: 27 - protein, 121 aa, Artificial Sequence
  7E3 Hu VH version 2-70
  misc_feature (30)..(30): Xaa may be Ser or Thr
  misc_feature (71)..(71): Xaa may be Ile or Phe
  misc_feature (98)..(98): Xaa may be Ile or Ala
QVTLRESGPALVKPTQTLTLTCTFSGFSLXTYNIGVGWIRQPPGKALEWLAHIWWNDNIYYNTVLKSRLTXSKDTSKNQVVLTMTNMDPVDTATYYCXRMAEGRYDAMDYWGQGTLVTVSS

SEQ ID NO: 28 - protein, 7 aa, Artificial Sequence
  7E3 Hu VH version 2-70 CDR1
TYNIGVG

SEQ ID NO: 29 - protein, 16 aa, Artificial Sequence
  7E3 Hu VH version 2-70 CDR2
HIWWNDNIYYNTVLKS

SEQ ID NO: 30 - protein, 11 aa, Artificial Sequence
  7E3 Hu VH version 2-70 CDR3
MAEGRYDAMDY

SEQ ID NO: 31 - protein, 121 aa, Artificial Sequence
  7E3 Hu VH version 3-66
  misc_feature (24)..(24): Xaa may be Phe or Ala
  misc_feature (50)..(50): Xaa may be Val or Leu
  misc_feature (71)..(71): Xaa may be Ile or Phe
  misc_feature (73)..(73): Xaa may be Lys or Arg
  misc_feature (80)..(80): Xaa may be Leu or Val
  misc_feature (98)..(98): Xaa may be Ile or Ala
EVQLVESGGGLVQPGGSLRLSCAXSGFSLTTYNIGVGWVRQAPGKGLEWXSHIWWNDNIYYNTVLKSRLTXSXDNSKNTXYLQMNSLRAEDTAVYYCXRMAEGRYDAMDYWGQGTLVTVSS

SEQ ID NO: 32 - protein, 107 aa, Artificial Sequence
  7E3 Hu VL version L19
  misc_feature (71)..(71): Xaa may be Phe or Tyr
  misc_feature (87)..(87): Xaa may be Tyr or Phe
DIQMTQSPSSVSASVGDRVTITCRASQDITNYLNWYQQKPGKAPKLLIYYTSKLHSGVPSRFSGSGSGTDXTLTISSLQPEDFATYXCQQGNTFPWTFGGGTKVEIK

SEQ ID NO: 33 - protein, 11 aa, Artificial Sequence
  7E3 Hu VL version L19 CDR1
RASQDITNYLN

SEQ ID NO: 34 - protein, 7 aa, Artificial Sequence
  7E3 Hu VL version L19 CDR2
YTSKLHS

SEQ ID NO: 35 - protein, 9 aa, Artificial Sequence
  7E3 Hu VL version L19 CDR3
QQGNTFPWT

SEQ ID NO: 36 - protein, 118 aa, Artificial Sequence; identical to SEQ ID NO: 7
  15C1 humanized VH mutant 1

SEQ ID NO: 37 - DNA, 354 bp, Artificial Sequence
  15C1 humanized VH mutant 1
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgacgcctttccttactggggccaagggactctggtcactgtctcttcc

SEQ ID NO: 38 - protein, 118 aa, Artificial Sequence
  15C1 humanized VH mutant 2
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDPSEGFPYWGQGTLVTVSS

SEQ ID NO: 39 - DNA, 354 bp, Artificial Sequence
  15C1 humanized VH mutant 2
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgagggatttccttactggggccaagggactctggtcactgtctcttcc

SEQ ID NO: 40 - protein, 107 aa, Artificial Sequence
  15C1 humanized VL mutant 1
EIVLTQSPDFQSVTPKEKVTITCRASQSISDHLHWYQQKPDQSPKLLIKYASHAISGVPSRFSGSGSGTDFTLTINSLEAEDAATYYCQNSHSFPLTFGGGTKVEIK

SEQ ID NO: 41 - DNA, 321 bp, Artificial Sequence
  15C1 humanized VL mutant 1
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagaatagtcacagttttccgctcactttcggcggagggaccaaggtggagatcaaa

SEQ ID NO: 42 - protein, 107 aa, Artificial Sequence; identical to SEQ ID NO: 8
  15C1 humanized VL mutant 2

SEQ ID NO: 43 - DNA, 321 bp, Artificial Sequence
  15C1 humanized VL mutant 2
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggtcacagttttccgctcactttcggcggagggaccaaggtggagatcaaa

SEQ ID NO: 44 - protein, 107 aa, Artificial Sequence
  15C1 humanized VL mutant 3
EIVLTQSPDFQSVTPKEKVTITCRASQSISDHLHWYQQKPDQSPKLLIKYASHAISGVPSRFSGSGSGTDFTLTINSLEAEDAATYYCQNSSSFPLTFGGGTKVEIK

SEQ ID NO: 45 - DNA, 321 bp, Artificial Sequence
  15C1 humanized VL mutant 3
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagaatagtagtagttttccgctcactttcggcggagggaccaaggtggagatcaaa

SEQ ID NO: 46 - protein, 107 aa, Artificial Sequence
  15C1 humanized VL mutant 4
EIVLTQSPDFQSVTPKEKVTITCRASQSISDHLHWYQQKPDQSPKLLIKYASHAISGVPSRFSGSGSGTDFTLTINSLEAEDAATYYCQQSHSFPLTFGGGTKVEIK

SEQ ID NO: 47 - DNA, 321 bp, Artificial Sequence
  15C1 humanized VL mutant 4
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagagtcacagttttccgctcactttcggcggagggaccaaggtggagatcaaa

SEQ ID NO: 48 - protein, 9 aa, Artificial Sequence
  anti-TLR4 variable heavy chain complementarity determining region 1
  misc_feature (2)..(2): Xaa may be Phe or Tyr
  misc_feature (5)..(5): Xaa may be Arg, Gly or Trp
  misc_feature (6)..(6): Xaa may be Tyr, Phe or Gly
GXPIXXGYS

SEQ ID NO: 49 - protein, 9 aa, Artificial Sequence
  anti-TLR4 variable heavy chain complementarity determining region 1
GYSITGGYS

SEQ ID NO: 50 - protein, 9 aa, Artificial Sequence
  anti-TLR4 variable heavy chain complementarity determining region 1
GFPIRYGYS

SEQ ID NO: 51 - protein, 9 aa, Artificial Sequence
  anti-TLR4 variable heavy chain complementarity determining region 1
GYPIRFGYS

SEQ ID NO: 52 - protein, 9 aa, Artificial Sequence
  anti-TLR4 variable heavy chain complementarity determining region 1
GYPIRHGYS

SEQ ID NO: 53 - protein, 9 aa, Artificial Sequence
  anti-TLR4 variable heavy chain complementarity determining region 1
GFPIGQGYS

SEQ ID NO: 54 - protein, 9 aa, Artificial Sequence
  anti-TLR4 variable heavy chain complementarity determining region 1
GYPIWGGYS

SEQ ID NO: 55 - protein, 9 aa, Artificial Sequence
  anti-TLR4 variable heavy chain complementarity determining region 1
GYPIGGGYS

SEQ ID NO: 56 - protein, 7 aa, Artificial Sequence
  anti-TLR4 variable heavy chain complementarity determining region 2
IHYSGYT

SEQ ID NO: 57 - protein, 11 aa, Artificial Sequence
  anti-TLR4 variable heavy chain complementarity determining region 3
  misc_feature (7)..(7): Xaa may be Asn, Gln, Asp or Glu
  misc_feature (8)..(9): Xaa may be any hydrophobic amino acid
ARKDSGXXXPY

SEQ ID NO: 58 - protein, 11 aa, Artificial Sequence
  anti-TLR4 variable heavy chain complementarity determining region 3
ARKDSGNYFPY

SEQ ID NO: 59 - protein, 11 aa, Artificial Sequence
  anti-TLR4 variable heavy chain complementarity determining region 3
ARKDSGRLLPY

SEQ ID NO: 60 - protein, 11 aa, Artificial Sequence
  anti-TLR4 variable heavy chain complementarity determining region 3
ARKDSGKWLPY

SEQ ID NO: 61 - protein, 11 aa, Artificial Sequence
  anti-TLR4 variable heavy chain complementarity determining region 3
ARKDSGHLMPY

SEQ ID NO: 62 - protein, 11 aa, Artificial Sequence
  anti-TLR4 variable heavy chain complementarity determining region 3
ARKDSGHNYPY

SEQ ID NO: 63 - protein, 11 aa, Artificial Sequence
  anti-TLR4 variable heavy chain complementarity determining region 3
ARKDSGKNFPY

SEQ ID NO: 64 - protein, 11 aa, Artificial Sequence
  anti-TLR4 variable heavy chain complementarity determining region 3
ARKDSGQLFPY

SEQ ID NO: 65 - protein, 11 aa, Artificial Sequence
  anti-TLR4 variable heavy chain complementarity determining region 3
ARKDSGHNLPY

SEQ ID NO: 66 - protein, 11 aa, Artificial Sequence
  anti-TLR4 variable heavy chain complementarity determining region 3
ARKDSGDYFPY

SEQ ID NO: 67 - protein, 11 aa, Artificial Sequence
  anti-TLR4 variable heavy chain complementarity determining region 3
ARKDSGRYWPY

SEQ ID NO: 68 - protein, 6 aa, Artificial Sequence
  anti-TLR4 variable light chain complementarity determining region 1
QSISDH

SEQ ID NO: 69 - protein, 3 aa, Artificial Sequence
  anti-TLR4 variable light chain complementarity determining region 2
YAS

SEQ ID NO: 70 - protein, 9 aa, Artificial Sequence
  anti-TLR4 variable light chain complementarity determining region 3
  misc_feature (4)..(4): Xaa may be Tyr or Asn
  misc_feature (5)..(5): Xaa may be Asp or Glu
  misc_feature (6)..(6): Xaa may be Phe or Tyr
  misc_feature (8)..(8): Xaa may be any hydrophobic amino acid
QQGXXXPXT

SEQ ID NO: 71 - protein, 9 aa, Artificial Sequence
  anti-TLR4 variable light chain complementarity determining region 3
QQGNDFPVT

SEQ ID NO: 72 - protein, 9 aa, Artificial Sequence
  anti-TLR4 variable light chain complementarity determining region 3
QQGYDEPFT

SEQ ID NO: 73 - protein, 9 aa, Artificial Sequence
  anti-TLR4 variable light chain complementarity determining region 3
QQGYDFPLT

SEQ ID NO: 74 - protein, 9 aa, Artificial Sequence
  anti-TLR4 variable light chain complementarity determining region 3
QQGYDYPLT

SEQ ID NO: 75 - protein, 9 aa, Artificial Sequence
  anti-TLR4 variable light chain complementarity determining region 3
QQGYEFPLT

SEQ ID NO: 76 - protein, 4 aa, Artificial Sequence
  altered human IgG1 gamma heavy chain constant region
SKAF

SEQ ID NO: 77 - protein, 826 aa, Cynomolgus sp.
MTSALRLAGTLIPAMAFLSCVRPESWEPCVEVVPNITYQCMELKFYKIPDNIPFSTKNLDLSFNPLRHLGSYSFLRFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQNIYCKDLQVLHQMPLSNLSLDLSLNPINFIQPGAFKEIRLHKLTLRSNFDDLNVMKTCIQGLAGLEVHRLVLGEFRNERNLEEFDKSSLEGLCNLTIEEFRLTYLDCYLDNIIDLFNCLANVSSFSLVSVNIKRVEDFSYNFRWQHLELVNCKFEQFPTLELKSLKRLTFTANKGGNAFSEVDLPSLEFLDLSRNGLSFKGCCSQSDFGTTSLKYLDLSFNDVITMSSNFLGLEQLEHLDFQHSNLKQMSQFSVFLSLRNLIYLDISHTHTRVAFNGIFDGLLSLKVLKMAGNSFQENFLPDIFTDLKNLTFLDLSQCQLEQLSPTAFDTLNKLQVLNMSHNNFFSLDTFPYKCLPSLQVLDYSLNHIMTSNNQELQHFPSSLAFLNLTQNDFACTCEHQSFLQWIKDQRQLLVEAERMECATPSDKQGMPVLSLNITCQMNKTIIGVSVFSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGQHIFWRRLRKALLDGKSWNPEEQ

SEQ ID NO: 78 - DNA, 354 bp, Artificial Sequence
  1E11 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 79 - protein, 118 aa, Artificial Sequence
  1E11 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 80 - DNA, 321 bp, Artificial Sequence; identical to SEQ ID NO: 43
  1E11 anti-TLR4 variable light chain

SEQ ID NO: 81 - protein, 107 aa, Artificial Sequence; identical to SEQ ID NO: 8
  1E11 anti-TLR4 variable light chain

SEQ ID NO: 82 - DNA, 354 bp, Artificial Sequence
  1A1 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattccggccgcctcctcccttactggggccaagggactctggtcactgtctcttcc

SEQ ID NO: 83 - protein, 118 aa, Artificial Sequence
  1A1 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGRLLPYWGQGTLVTVSS

SEQ ID NO: 84 - DNA, 321 bp, Artificial Sequence; identical to SEQ ID NO: 43
  1A1 anti-TLR4 variable light chain

SEQ ID NO: 85 - protein, 107 aa, Artificial Sequence; identical to SEQ ID NO: 8
  1A1 anti-TLR4 variable light chain

SEQ ID NO: 86 - DNA, 354 bp, Artificial Sequence
  1A6 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggcaagtggttgccttactggggccaagggactctggtcactgtctcttcc

SEQ ID NO: 87 - protein, 118 aa, Artificial Sequence
  1A6 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGKWLPYWGQGTLVTVSS

SEQ ID NO: 88 - DNA, 321 bp, Artificial Sequence; identical to SEQ ID NO: 43
  1A6 anti-TLR4 variable light chain

SEQ ID NO: 89 - protein, 107 aa, Artificial Sequence; identical to SEQ ID NO: 8
  1A6 anti-TLR4 variable light chain

SEQ ID NO: 90 - DNA, 354 bp, Artificial Sequence
  1B12 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcgggcacctcatgccttactggggccaagggactctggtcactgtctcttcc

SEQ ID NO: 91 - protein, 118 aa, Artificial Sequence
  1B12 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGHLMPYWGQGTLVTVSS

SEQ ID NO: 92 - DNA, 321 bp, Artificial Sequence; identical to SEQ ID NO: 43
  1B12 anti-TLR4 variable light chain

SEQ ID NO: 93 - protein, 107 aa, Artificial Sequence; identical to SEQ ID NO: 8
  1B12 anti-TLR4 variable light chain

SEQ ID NO: 94 - DNA, 354 bp, Artificial Sequence
  1C7 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattccgggcacaactacccttactggggccaagggactctggtcactgtctcttcc

SEQ ID NO: 95 - protein, 118 aa, Artificial Sequence
  1C7 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGHNYPYWGQGTLVTVSS

SEQ ID NO: 96 - DNA, 321 bp, Artificial Sequence; identical to SEQ ID NO: 43
  1C7 anti-TLR4 variable light chain

SEQ ID NO: 97 - protein, 107 aa, Artificial Sequence; identical to SEQ ID NO: 8
  1C7 anti-TLR4 variable light chain

SEQ ID NO: 98 - DNA, 354 bp, Artificial Sequence
  1C10 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggcaagaacttcccttactggggccaagggactctggtcactgtctcttcc

SEQ ID NO: 99 - protein, 118 aa, Artificial Sequence
  1C10 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGKNFPYWGQGTLVTVSS

SEQ ID NO: 100 - DNA, 321 bp, Artificial Sequence; identical to SEQ ID NO: 43
  1C10 anti-TLR4 variable light chain

SEQ ID NO: 101 - protein, 107 aa, Artificial Sequence; identical to SEQ ID NO: 8
  1C10 anti-TLR4 variable light chain

SEQ ID NO: 102 - DNA, 354 bp, Artificial Sequence
  1C12 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggccagttgttcccttactggggccaagggactctggtcactgtctcttcc

SEQ ID NO: 103 - protein, 118 aa, Artificial Sequence
  1C12 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGQLFPYWGQGTLVTVSS

SEQ ID NO: 104 - DNA, 321 bp, Artificial Sequence; identical to SEQ ID NO: 43
  1C12 anti-TLR4 variable light chain

SEQ ID NO: 105 - protein, 107 aa, Artificial Sequence; identical to SEQ ID NO: 8
  1C12 anti-TLR4 variable light chain

SEQ ID NO: 106 - DNA, 354 bp, Artificial Sequence
  1D10 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggccacaacttgccttactggggccaagggactctggtcactgtctcttcc

SEQ ID NO: 107 - protein, 118 aa, Artificial Sequence
  1D10 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGHNLPYWGQGTLVTVSS

SEQ ID NO: 108 - DNA, 321 bp, Artificial Sequence; identical to SEQ ID NO: 43
  1D10 anti-TLR4 variable light chain

SEQ ID NO: 109 - protein, 107 aa, Artificial Sequence; identical to SEQ ID NO: 8
  1D10 anti-TLR4 variable light chain

SEQ ID NO: 110 - DNA, 354 bp, Artificial Sequence
  1E11 N103D anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcgactacttcccttactggggccaagggactctggtcactgtctcttcc

SEQ ID NO: 111 - protein, 118 aa, Artificial Sequence
  1E11 N103D anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGDYFPYWGQGTLVTVSS

SEQ ID NO: 112 - DNA, 321 bp, Artificial Sequence; identical to SEQ ID NO: 43
  1E11 N103D anti-TLR4 variable light chain

SEQ ID NO: 113 - protein, 107 aa, Artificial Sequence; identical to SEQ ID NO: 8
  1E11 N103D anti-TLR4 variable light chain

SEQ ID NO: 114 - DNA, 354 bp, Artificial Sequence
  1G12 anti-TLR4 variable heavy chain
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattccgggcggtactggccttactggggccaagggactctggtcactgtctcttcc

SEQ ID NO: 115 - protein, 118 aa, Artificial Sequence
  1G12 anti-TLR4 variable heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGRYWPYWGQGTLVTVSS

SEQ ID NO: 116 - DNA, 321 bp, Artificial Sequence; identical to SEQ ID NO: 43
  1G12 anti-TLR4 variable light chain

SEQ ID NO: 117 - protein, 107 aa, Artificial Sequence; identical to SEQ ID NO: 8
  1G12 anti-TLR4 variable light chain

SEQ ID NO: 118 - DNA, 354 bp, Artificial Sequence
  1E11.C1 anti-TLR4 variable heavy chain
caggtgcagcttcaggagtccggcccaggactggtgaagccttcggacaccctgtccctcacctgcgctgtctctggtttcccgatccgctacgggtatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 119 - protein, 118 aa, Artificial Sequence
  1E11.C1 anti-TLR4 variable heavy chain
QVQLQESGPGLVKPSDTLSLTCAVSGFPIRYGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 120 - DNA, 321 bp, Artificial Sequence; identical to SEQ ID NO: 43
  1E11.C1 anti-TLR4 variable light chain

SEQ ID NO: 121 - protein, 107 aa, Artificial Sequence; identical to SEQ ID NO: 8
  1E11.C1 anti-TLR4 variable light chain

SEQ ID NO: 122 - DNA, 354 bp, Artificial Sequence
  1E11.C2 anti-TLR4 variable heavy chain
caggtgcagcttcaggagtccggcccaggactggtgaagccttcggacaccctgtccctcacctgcgctgtctctggttacccgatccggttcggctatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 123 - protein, 118 aa, Artificial Sequence
  1E11.C2 anti-TLR4 variable heavy chain
QVQLQESGPGLVKPSDTLSLTCAVSGYPIRFGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 124 - DNA, 321 bp, Artificial Sequence; identical to SEQ ID NO: 43
  1E11.C2 anti-TLR4 variable light chain

SEQ ID NO: 125 - protein, 107 aa, Artificial Sequence; identical to SEQ ID NO: 8
  1E11.C2 anti-TLR4 variable light chain

SEQ ID NO: 126 - DNA, 354 bp, Artificial Sequence
  1E11.C3 anti-TLR4 variable heavy chain
caggtgcagcttcaggagtccggcccaggactggtgaagccttcggacaccctgtccctcacctgcgctgtctctggttaccccatccggcacgggtacagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 127 - protein, 118 aa, Artificial Sequence
  1E11.C3 anti-TLR4 variable heavy chain
QVQLQESGPGLVKPSDTLSLTCAVSGYPIRHGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 128 - DNA, 321 bp, Artificial Sequence; identical to SEQ ID NO: 43
  1E11.C3 anti-TLR4 variable light chain

SEQ ID NO: 129 - protein, 107 aa, Artificial Sequence; identical to SEQ ID NO: 8
  1E11.C3 anti-TLR4 variable light chain

SEQ ID NO: 130 - DNA, 354 bp, Artificial Sequence
  1E11.C4 anti-TLR4 variable heavy chain
caggtgcagcttcaggagtccggcccaggactggtgaagccttcggacaccctgtccctcacctgcgctgtctctggtttcccgatcggccaggggtatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 131 - protein, 118 aa, Artificial Sequence
  1E11.C4 anti-TLR4 variable heavy chain
QVQLQESGPGLVKPSDTLSLTCAVSGFPIGQGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 132 - DNA, 321 bp, Artificial Sequence; identical to SEQ ID NO: 43
  1E11.C4 anti-TLR4 variable light chain

SEQ ID NO: 133 - protein, 107 aa, Artificial Sequence; identical to SEQ ID NO: 8
  1E11.C4 anti-TLR4 variable light chain

SEQ ID NO: 134 - DNA, 363 bp, Artificial Sequence
  1E11.C5 anti-TLR4 variable heavy chain
caggtgcagcttcaggagtccggcccaggactggtgaagccttcggacaccctgtccctcacctgcgctgtctctggttacccgatctgggggggctatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttccgcctccacc

SEQ ID NO: 135 - protein, 118 aa, Artificial Sequence
  1E11.C5 anti-TLR4 variable heavy chain
QVQLQESGPGLVKPSDTLSLTCAVSGYPIWGGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 136 - DNA, 321 bp, Artificial Sequence; identical to SEQ ID NO: 43
  1E11.C5 anti-TLR4 variable light chain

SEQ ID NO: 137 - protein, 107 aa, Artificial Sequence; identical to SEQ ID NO: 8
  1E11.C5 anti-TLR4 variable light chain

SEQ ID NO: 138 - DNA, 354 bp, Artificial Sequence
  1E11.C6 anti-TLR4 variable heavy chain
caggtgcagcttcaggagtccggcccaggactggtgaagccttcggacaccctgtccctcacctgcgctgtctctggttaccccatcggcggcggctatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 139 - protein, 118 aa, Artificial Sequence
  1E11.C6 anti-TLR4 variable heavy chain
QVQLQESGPGLVKPSDTLSLTCAVSGYPIGGGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 140 - DNA, 321 bp, Artificial Sequence; identical to SEQ ID NO: 43
  1E11.C6 anti-TLR4 variable light chain

SEQ ID NO: 141 - protein, 107 aa, Artificial Sequence; identical to SEQ ID NO: 8
  1E11.C6 anti-TLR4 variable light chain

SEQ ID NO: 142 - DNA, 354 bp, Artificial Sequence; identical to SEQ ID NO: 78
  1E11.E1 anti-TLR4 variable heavy chain

SEQ ID NO: 143 - protein, 118 aa, Artificial Sequence; identical to SEQ ID NO: 79
  1E11.E1 anti-TLR4 variable heavy chain

SEQ ID NO: 144 - DNA, 321 bp, Artificial Sequence
  1E11.E1 anti-TLR4 variable light chain
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcaggggaacgacttcccggtgactttcggcggagggaccaaggtggagatcaaa

SEQ ID NO: 145 - protein, 107 aa, Artificial Sequence
  1E11.E1 anti-TLR4 variable light chain
EIVLTQSPDFQSVTPKEKVTITCRASQSISDHLHWYQQKPDQSPKLLIKYASHAISGVPSRFSGSGSGTDFTLTINSLEAEDAATYYCQQGNDFPVTFGGGTKVEIK

SEQ ID NO: 146 - DNA, 354 bp, Artificial Sequence; identical to SEQ ID NO: 78
  1E11.E2 anti-TLR4 variable heavy chain

SEQ ID NO: 147 - protein, 118 aa, Artificial Sequence; identical to SEQ ID NO: 79
  1E11.E2 anti-TLR4 variable heavy chain

SEQ ID NO: 148 - DNA, 321 bp, Artificial Sequence
  1E11.E2 anti-TLR4 variable light chain
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcaggggtacgacgagccgttcactttcggcggagggaccaaggtggagatcaaa

SEQ ID NO: 149 - protein, 107 aa, Artificial Sequence
  1E11.E2 anti-TLR4 variable light chain
EIVLTQSPDFQSVTPKEKVTITCRASQSISDHLHWYQQKPDQSPKLLIKYASHAISGVPSRFSGSGSGTDFTLTINSLEAEDAATYYCQQGYDEPFTFGGGTKVEIK

SEQ ID NO: 150 - DNA, 354 bp, Artificial Sequence; identical to SEQ ID NO: 78
  1E11.E3 anti-TLR4 variable heavy chain

SEQ ID NO: 151 - protein, 118 aa, Artificial Sequence; identical to SEQ ID NO: 79
  1E11.E3 anti-TLR4 variable heavy chain

SEQ ID NO: 152 - DNA, 321 bp, Artificial Sequence
  1E11.E3 anti-TLR4 variable light chain
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggctacgacttcccgttgactttcggcggagggaccaaggtggagatcaaa

SEQ ID NO: 153 - protein, 107 aa, Artificial Sequence
  1E11.E3 anti-TLR4 variable light chain
EIVLTQSPDFQSVTPKEKVTITCRASQSISDHLHWYQQKPDQSPKLLIKYASHAISGVPSRFSGSGSGTDFTLTINSLEAEDAATYYCQQGYDFPLTFGGGTKVEIK

SEQ ID NO: 154 - DNA, 354 bp, Artificial Sequence; identical to SEQ ID NO: 78
  1E11.E4 anti-TLR4 variable heavy chain

SEQ ID NO: 155 - protein, 118 aa, Artificial Sequence; identical to SEQ ID NO: 79
  1E11.E4 anti-TLR4 variable heavy chain

SEQ ID NO: 156 - DNA, 321 bp, Artificial Sequence
  1E11.E4 anti-TLR4 variable light chain
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggctacgactacccgctcactttcggcggagggaccaaggtggagatcaaa

SEQ ID NO: 157 - protein, 107 aa, Artificial Sequence
  1E11.E4 anti-TLR4 variable light chain
EIVLTQSPDFQSVTPKEKVTITCRASQSISDHLHWYQQKPDQSPKLLIKYASHAISGVPSRFSGSGSGTDFTLTINSLEAEDAATYYCQQGYDYPLTFGGGTKVEIK

SEQ ID NO: 158 - DNA, 354 bp, Artificial Sequence; identical to SEQ ID NO: 78
  1E11.E5 anti-TLR4 variable heavy chain

SEQ ID NO: 159 - protein, 118 aa, Artificial Sequence; identical to SEQ ID NO: 79
  1E11.E5 anti-TLR4 variable heavy chain

SEQ ID NO: 160 - DNA, 321 bp, Artificial Sequence
  1E11.E5 anti-TLR4 variable light chain
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggctacgagttcccgttgactttcggcggagggaccaaggtggagatcaaa

SEQ ID NO: 161 - protein, 107 aa, Artificial Sequence
  1E11.E5 anti-TLR4 variable light chain
EIVLTQSPDFQSVTPKEKVTITCRASQSISDHLHWYQQKPDQSPKLLIKYASHAISGVPSRFSGSGSGTDFTLTINSLEAEDAATYYCQQGYEFPLTFGGGTKVEIK

SEQ ID NO: 162 - DNA, 354 bp, Artificial Sequence; identical to SEQ ID NO: 122
  1E11.C2E1 anti-TLR4 variable heavy chain

SEQ ID NO: 163 - protein, 118 aa, Artificial Sequence; identical to SEQ ID NO: 123
  1E11.C2E1 anti-TLR4 variable heavy chain

SEQ ID NO: 164 - DNA, 321 bp, Artificial Sequence; identical to SEQ ID NO: 144
  1E11.C2E1 anti-TLR4 variable light chain

SEQ ID NO: 165 - protein, 107 aa, Artificial Sequence; identical to SEQ ID NO: 145
  1E11.C2E1 anti-TLR4 variable light chain

SEQ ID NO: 166 - DNA, 354 bp, Artificial Sequence; identical to SEQ ID NO: 122
  1E11.C2E3 anti-TLR4 variable heavy chain

SEQ ID NO: 167 - protein, 118 aa, Artificial Sequence; identical to SEQ ID NO: 123
  1E11.C2E3 anti-TLR4 variable heavy chain

SEQ ID NO: 168 - DNA, 321 bp, Artificial Sequence; identical to SEQ ID NO: 152
  1E11.C2E3 anti-TLR4 variable light chain

SEQ ID NO: 169 - protein, 107 aa, Artificial Sequence; identical to SEQ ID NO: 153
  1E11.C2E3 anti-TLR4 variable light chain

SEQ ID NO: 170 - DNA, 354 bp, Artificial Sequence; identical to SEQ ID NO: 122
  1E11.C2E4 anti-TLR4 variable heavy chain

SEQ ID NO: 171 - protein, 118 aa, Artificial Sequence; identical to SEQ ID NO: 123
  1E11.C2E4 anti-TLR4 variable heavy chain

SEQ ID NO: 172 - DNA, 321 bp, Artificial Sequence; identical to SEQ ID NO: 156
  1E11.C2E4 anti-TLR4 variable light chain

SEQ ID NO: 173 - protein, 107 aa, Artificial Sequence; identical to SEQ ID NO: 157
  1E11.C2E4 anti-TLR4 variable light chain

SEQ ID NO: 174 - DNA, 354 bp, Artificial Sequence; identical to SEQ ID NO: 122
  1E11.C2E5 anti-TLR4 variable heavy chain

SEQ ID NO: 175 - protein, 118 aa, Artificial Sequence; identical to SEQ ID NO: 123
  1E11.C2E5 anti-TLR4 variable heavy chain

SEQ ID NO: 176 - DNA, 321 bp, Artificial Sequence; identical to SEQ ID NO: 160
  1E11.C2E5 anti-TLR4 variable light chain

SEQ ID NO: 177 - protein, 107 aa, Artificial Sequence; identical to SEQ ID NO: 161
  1E11.C2E5 anti-TLR4 variable light chain

SEQ ID NO: 178 - protein, 447 aa, Artificial Sequence
  1E11 IgG1 formatted anti-TLR4 heavy chain
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGNYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 214 aa, Artificial Sequence
  1E11 IgG1 formatted anti-TLR4 light chain
EIVLTQSPDFQSVTPKEKVTITCRASQSISDHLHWYQQKPDQSPKLLIKYASHAISGVPSRFSGSGSGTDFTLTINSLEAEDAATYYCQQGHSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - DNA, 705 bp, Artificial Sequence
  1E11 IgG1 formatted anti-TLR4 light chain
atgagtgtgcccactcaggtcctggggttgctgctgctgtggcttacagatgccagatgtgaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggtcacagttttccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 181 - DNA, 1398 bp, Artificial Sequence
  1E11 IgG1 formatted anti-TLR4 heavy chain
atggaatggagctgggtctttctcttcttcctgtcagtaactacaggtgtccaccaggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgacgcctttccttactggggccaagggactctggtcactgtctcttccgcctccaccaagggcccatcggtcttccccctggcaccctcctccaagagcacctctgggggcacagcggccctgggctgcctggtcaaggactacttccccgaaccggtgacagtctcgtggaactcaggagccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatctcgggaggagatgaccaagaaccaggtcagcctgacttgcctggtcaaaggcttctatcccagcgacatcgccgtggagtgggagagcaacgggcagccggagaacaactacaagaccacgcctcccgtgctggactccgacggctccttcttcctctatagcaagctcaccgtggacaagtccaggtggcagcaggggaacgtcttctcatgctccgtgatgcatgaggctctgcacaaccactacacgcagaagagcctctccctgtctccgggttaa

SEQ ID NO: 182 - protein, 214 aa, Artificial Sequence; identical to SEQ ID NO: 179
  1E11.C1 IgG1 formatted anti-TLR4 light chain

SEQ ID NO: 183 - protein, 446 aa, Artificial Sequence
  1E11.C1 IgG1 formatted anti-TLR4 heavy chain
QVQLQESGPGLVKPSDTLSLTCAVSGFPIRYGYSWHWIRQPPGKGLEWMGYIHYSGYTDFNPLKTRITISRDTSKNQFSLKLSSVTAVDTAVYYCARKDSGNYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - DNA, 705 bp, Artificial Sequence; identical to SEQ ID NO: 180
  1E11.C1 IgG1 formatted anti-TLR4 light chain

SEQ ID NO: 185 - DNA, 1398 bp, Artificial Sequence
  1E11.C1 IgG1 formatted anti-TLR4 heavy chain
atggaatggagctgggtctttctcttcttcctgtcagtaactacaggtgtccaccaggtgcagcttcaggagtccggcccaggactggtgaagccttcggacaccctgtccctcacctgcgctgtctctggtttcccgatccgctacgggtatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttccgcctccaccaagggcccatcggtcttccccctggcaccctcctccaagagcacctctgggggcacagcggccctgggctgcctggtcaaggactacttccccgaaccggtgacagtctcgtggaactcaggagccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatctcgggaggagatgaccaagaaccaggtcagcctgacttgcctggtcaaaggcttctatcccagcgacatcgccgtggagtgggagagcaacgggcagccggagaacaactacaagaccacgcctcccgtgctggactccgacggctccttcttcctctatagcaagctcaccgtggacaagtccaggtggcagcaggggaacgtcttctcatgctccgtgatgcatgaggctctgcacaaccactacacgcagaagagcctctccctgtctccgggttaa

SEQ ID NO: 186 - protein, 9 aa, Artificial Sequence
  VH CDR3
KDPSDAFPY

SEQ ID NO: 187 - protein, 9 aa, Artificial Sequence
  VH CDR3
KDPSEGFPY

SEQ ID NO: 188 - protein, 9 aa, Artificial Sequence
  VL CDR3
QNSHSFPLT

SEQ ID NO: 189 - protein, 9 aa, Artificial Sequence
  VL CDR3
QQGHSFPLT

SEQ ID NO: 190 - protein, 9 aa, Artificial Sequence
  VL CDR3
QNSSSFPLT

SEQ ID NO: 191 - protein, 8 aa, Artificial Sequence
  VL CDR3
QQSHSFPL

SEQ ID NO: 192 - protein, 4 aa, Artificial Sequence
  N 325 S; K 326 A
SAAF

SEQ ID NO: 193 - protein, 4 aa, Artificial Sequence
  N 325 S; L 328 F
SKAF

SEQ ID NO: 194 - protein, 4 aa, Artificial Sequence
  K 326 A; L 328 F
NAAF

SEQ ID NO: 195 - protein, 4 aa, Artificial sequence
  L 328 F
NKAF

SEQ ID NO: 196 - protein, 4 aa, Homo sapiens
NKAL

SEQ ID NO: 197 - DNA, 357 bp, Artificial Sequence
  chemically synthesized
caggtgcagctggtggagtctgggggaggcgtggtccagcctgggaggtccctgagactctcctgtgcagcctctggattcaccttcagtaactctggcatacactgggtccgccaggctccaggcaagggactggagtgggtggcagttatatcatatgatggaagtaacaaatactacgcagactccgtgaagggccgattcaccatctccagagacaactccaagaacactctgtatctgcaaatgaacagcctgagagctgaggacacggctgtctattattgtgcaagattgagggataatgcggagtatactgattactggggccagggaaccctggtcaccgtctcgagt

SEQ ID NO: 198 - protein, 119 aa, Artificial Sequence
  chemically synthesized
QVQLVESGGGVVQPGRSLRLSCAASGFTFSNSGIHWVRQAPGKGLEWVAVISYDGSNKYYADSVKGRFTISRDNSKNTLYLQMNSLRAEDTAVYYCARLRDNAEYTDYWGQGTLVTVSS

SEQ ID NO: 199 - DNA, 334 bp, Artificial Sequence
  chemically synthesized
aattttatgctgactcagccccactctgtgtcggagtctccggggaagacggtaaccatctcctgcaccggcagcggtggcagcattgccagcaactatgtgcagtggtaccaacagcgcccgggcagttcccccaccactgtcatctatgaggataaccagagaccctctggggtccctgatcggttctctggctccatcgacagctcctccaattctgcctccctcaccatctctgggctgaagactgaggacgaggctgactactactgtcagtcttatgatccgcttccggtgtgggttttcggcggagggaccaagctgaccgtcctag

SEQ ID NO: 200 - protein, 111 aa, Artificial Sequence
  chemically synthesized
NFMLTQPHSVSESPGKTVTISCTGSGGSIASNYVQWYQQRPGSSPTTVIYEDNQRPSGVPDRFSGSIDSSSNSASLTISGLKTEDEADYYCQSYDPLPVWVFGGGTKLTVL

SEQ ID NO: 201 - protein, 5 aa, Artificial Sequence
  chemically synthesized
NSGIH

SEQ ID NO: 202 - protein, 17 aa, Artificial Sequence
  chemically synthesized
VISYDGSNKYYADSVKG

SEQ ID NO: 203 - protein, 10 aa, Artificial Sequence
  chemically synthesized
LRDNAEYTDY

SEQ ID NO: 204 - protein, 13 aa, Artificial Sequence
  chemically synthesized
TGSGGSIASNYVQ

SEQ ID NO: 205 - protein, 7 aa, Artificial Sequence
  chemically synthesized
EDNQRPS

SEQ ID NO: 206 - protein, 10 aa, Artificial Sequence
  chemically synthesized
QSYDPLPVWV

SEQ ID NO: 207 - DNA, 358 bp, Artificial Sequence
  chemically synthesized
caggtgcagctggtggagtctgggggaggcgtggtccagcctgggaggtccctgagactctcctgtgcagcctctggattcaccttcagtaactctggcatacactgggtccgccaggctccaggcaagggactggagtgggtggcagttatatcatatgatggaagtaacaaattctacgcagactccgtgaagggccgattcaccatctccagagacaactccaagaacactctgtatctgcaaatgaacagcctgagagctgaggacacggctgtctattattgtgcaaaattgagggataatgcggagtatactgattactggggccagggaaccctggtcaccgtctcgagtg

SEQ ID NO: 208 - protein, 119 aa, Artificial Sequence
  chemically synthesized
QVQLVESGGGVVQPGRSLRLSCAASGFTFSNSGIHWVRQAPGKGLEWVAVISYDGSNKFYADSVKGRFTISRDNSKNTLYLQMNSLRAEDTAVYYCAKLRDNAEYTDYWGQGTLVTVSS

SEQ ID NO: 209 - DNA, 333 bp, Artificial Sequence
  chemically synthesized
aattttatgctgactcagccccactctgtgtcggagtctccggggaagacggtgaccatctcctgcaccggcagcggtggcagcattgacagaaactatgtgcagtggtaccagcagcgcccgggcagttcccccaccactgtgatctatgaggataaccaaagaccctctggggtcccggatcgattctctggctccatcgacagctcctccaactctgcctccctcaccatctctggactaaaaactgaagacgaggctgactactactgtcagtcttatgatccgcttccggtgtgggttttcggcggagggaccaagctcaccgtccta

SEQ ID NO: 210 - protein, 111 aa, Artificial Sequence
  chemically synthesized
NFMLTQPHSVSESPGKTVTISCTGSGGSIDRNYVQWYQQRPGSSPTTVIYEDNQRPSGVPDRFSGSIDSSSNSASLTISGLKTEDEADYYCQSYDPLPVWVFGGGTKLTVL

SEQ ID NO: 211 - protein, 17 aa, Artificial Sequence
  chemically synthesized
VISYDGSNKFYADSVKG

SEQ ID NO: 212 - protein, 13 aa, Artificial Sequence
  chemically synthesized
TGSGGSIDRNYVQ

SEQ ID NO: 213 - DNA, 357 bp, Artificial Sequence
  chemically synthesized
caggtgcagctggtggagtctgggggaggcgtggtccagcctgggaggtccctgagactctcctgtgcagcctctggattcaccttcagtaactctggcatacactgggtccgccaggctccaggcaagggactggagtgggtggcagttatatcatatgatggaagtaacaaattctacgcagactccgtgaagggccgattcaccatctccagagacaactccaagaacactctgtatctgcaaatgaacagcctgagagctgaggacacggctgtctattattgtgcaaaattgagggataatggtgagtacttagactactggggccagggaaccctggtcaccgtctcgagt

SEQ ID NO: 214 - protein, 119 aa, Artificial Sequence
  chemically synthesized
QVQLVESGGGVVQPGRSLRLSCAASGFTFSNSGIHWVRQAPGKGLEWVAVISYDGSNKFYADSVKGRFTISRDNSKNTLYLQMNSLRAEDTAVYYCAKLRDNGEYLDYWGQGTLVTVSS

SEQ ID NO: 215 - DNA, 333 bp, Artificial Sequence; identical to SEQ ID NO: 209
  chemically synthesized

SEQ ID NO: 216 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAR

SEQ ID NO: 217 - protein, 10 aa, Artificial Sequence
  chemically synthesized
LRDNGEYLDY

SEQ ID NO: 218 - DNA, 334 bp, Artificial Sequence
  chemically synthesized
aattttatgctgactcagccccactctgtgtcggagtctccggggaagacggtgaccatctcctgcaccggcagcggtggcagcattgacagaaactatgtgcagtggtaccagcagcgcccgggcagttcccccaccactgtgatctatgaggataaccaaagaccctctggggtcccggatcgattctctggctccatcgacagctcctccaactctgcctccctcaccatctctggactaaaaactgaagacgaggctgactactactgtcagtcttatgtggagacgcctgagtgggttttcggcggagggaccaagctcaccgtcctag

SEQ ID NO: 219 - protein, 111 aa, Artificial Sequence
  chemically synthesized
NFMLTQPHSVSESPGKTVTISCTGSGGSIDRNYVQWYQQRPGSSPTTVIYEDNQRPSGVPDRFSGSIDSSSNSASLTISGLKTEDEADYYCQSYVETPEWVFGGGTKLTVL

SEQ ID NO: 220 - protein, 10 aa, Artificial Sequence
  chemically synthesized
QSYVETPEWV

SEQ ID NO: 221 - DNA, 357 bp, Artificial Sequence
  chemically synthesized
caggtgcagctggtggagtctgggggaggcgtggtccagcctgggaggtcccacacactctcctgtgcagcctctggattcgccttcaaaaactctggcatacactgggtccgccaggctccaggcaagggactggagtgggtggcagttatatcatatgatggaagtaacaaattctacgcagactccgtgaagggccgattcaccatctccagagacaactcccagaacactgtatatctgcaaatgactgacctgagacctgacgacacggctgtctattattgtgcaagagatgggagtgagagcgagtacttagactactggggcaagggaaccctggtcaccgtctcgagt

SEQ ID NO: 222 - protein, 119 aa, Artificial Sequence
  chemically synthesized
QVQLVESGGGVVQPGRSHTLSCAASGFAFKNSGIHWVRQAPGKGLEWVAVISYDGSNKFYADSVKGRFTISRDNSQNTVYLQMTDLRPDDTAVYYCARDGSESEYLDYWGKGTLVTVSS

SEQ ID NO: 223 - DNA, 335 bp, Artificial Sequence
  chemically synthesized
aattttatgctgactcagccccactctgtgtcggagtctccggggaagacggtgaccatctcctgcaccggcagcggtggcagcattgacagaaactatgtgcagtggtaccagcagcgcccgggcagtgcccccatcactgtgatctatgaggataaccaaagaccctctggggtcccggatcgattctctggctccatcgacagctcctccaactctgcctccctcaccatctctggactacggactgacgacgaggctgactactactgtcagtcttatgatagcatcaatctttgggttttcggcggagggaccaaggtcaccgtcctagg

SEQ ID NO: 224 - protein, 111 aa, Artificial Sequence
  chemically synthesized
NFMLTQPHSVSESPGKTVTISCTGSGGSIDRNYVQWYQQRPGSAPITVIYEDNQRPSGVPDRFSGSIDSSSNSASLTISGLRTDDEADYYCQSYDSINLWVFGGGTKVTVL

SEQ ID NO: 225 - protein, 10 aa, Artificial Sequence
  chemically synthesized
QSYDSINLWV

SEQ ID NO: 226 - DNA, 360 bp, Artificial Sequence
  chemically synthesized
caggtgcagctggtgcagtttgggggaggcgtggtccagcctgggaggtccttgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagaaaatgccaagaactccgtgtatctgcaaatggacagcctgagagtcggggacacggctgtgtattactgtacaagagccgggtatagtactgactggcatcccgactactggggccaggggacaatggtcaccgtctcgagt

SEQ ID NO: 227 - protein, 120 aa, Artificial Sequence
  chemically synthesized
QVQLVQFGGGVVQPGRSLRLSCAASGFTFSSYGMHWVRQAPGKGLEWVAVISYDGSIKYYADSVKGRFTISRENAKNSVYLQMDSLRVGDTAVYYCTRAGYSTDWHPDYWGQGTMVTVSS

SEQ ID NO: 228 - DNA, 324 bp, Artificial Sequence
  chemically synthesized
tcttctgagctgactcaggaccctgctgtgtctgtggccttgggacagacagtcaggatcacatgccaaggagacagcctcagaagctattatgcaagctggtaccagcagaagccaggacaggcccctgtacttgccatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtccta

SEQ ID NO: 229 - protein, 108 aa, Artificial Sequence
  chemically synthesized
SSELTQDPAVSVALGQTVRITCQGDSLRSYYASWYQQKPGQAPVLAIYGKNNRPSGIPDRFSGSSSGNTASLTITGAQAEDEADYYCNSRDSSGNHVVFGGGTKLTVL

SEQ ID NO: 230 - protein, 5 aa, Artificial Sequence
  chemically synthesized
SYGMH

SEQ ID NO: 231 - protein, 17 aa, Artificial Sequence
  chemically synthesized
VISYDGSIKYYADSVKG

SEQ ID NO: 232 - protein, 11 aa, Artificial Sequence
  chemically synthesized
AGYSTDWHPDY

SEQ ID NO: 233 - protein, 11 aa, Artificial Sequence
  chemically synthesized
QGDSLRSYYAS

SEQ ID NO: 234 - protein, 7 aa, Artificial Sequence
  chemically synthesized
GKNNRPS

SEQ ID NO: 235 - protein, 11 aa, Artificial Sequence
  chemically synthesized
NSRDSSGNHVV

SEQ ID NO: 236 - DNA, 366 bp, Artificial Sequence
  chemically synthesized
caggtcaccttgagggagtctggtcctgcgctggtgaaacccacacagaccctcacactgacctgcaccttctctggattctcactcaccactagtggaatgtctgtgatttggatccgtcagcccccagggaaggccctggagtggcttgcacgcattgattcggatgacgagaaacactacaacacatctctgaagaccaggctcgccatctccaaggacacctccaaaaaccaggtggtccttacaatgaccaacatggaccctgtggacacaggcacctattactgtgcacggcttcgggctggttcaggtccatatgtttttgactcctgggggcaagggaccacggtcaccgtctcgagt

SEQ ID NO: 237 - protein, 122 aa, Artificial Sequence
  chemically synthesized
QVTLRESGPALVKPTQTLTLTCTFSGFSLTTSGMSVIWIRQPPGKALEWLARIDSDDEKHYNTSLKTRLAISKDTSKNQVVLTMTNMDPVDTGTYYCARLRAGSGPYVFDSWGQGTTVTVSS

SEQ ID NO: 238 - DNA, 331 bp, Artificial Sequence
  chemically synthesized
cagtctgtgctgactcagccaccctcagcgtctgggacccccgggcagagggtcaccatctcttgttctggaagcagctccaacatcgggagtaacactgtaaactggtaccagcgactcccaggagcggccccccaactcctcatctacaataatgaccagcggccctcagggatccctgaccgattctctggctccaagtctggcacctcaggctccctggtcatcagtgggctccagtctgaagacgaggctgattactactgtgcgtcatgggatgacagtctgaatggtcgggtgttcggcggagggaccaagctgaccgtcctag

SEQ ID NO: 239 - protein, 110 aa, Artificial Sequence
  chemically synthesized
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVL

SEQ ID NO: 240 - protein, 7 aa, Artificial Sequence
  chemically synthesized
TSGMSVI

SEQ ID NO: 241 - protein, 16 aa, Artificial Sequence
  chemically synthesized
RIDSDDEKHYNTSLKT

SEQ ID NO: 242 - protein, 12 aa, Artificial Sequence
  chemically synthesized
LRAGSGPYVFDS

SEQ ID NO: 243 - protein, 13 aa, Artificial Sequence
  chemically synthesized
SGSSSNIGSNTVN

SEQ ID NO: 244 - protein, 7 aa, Artificial Sequence
  chemically synthesized
NNDQRPS

SEQ ID NO: 245 - protein, 11 aa, Artificial Sequence
  chemically synthesized
ASWDDSLNGRV

SEQ ID NO: 246 - DNA, 369 bp, Artificial Sequence
  chemically synthesized
caggtgcagctggtggagtctgggggaggcgtggtccggcctgggaggtccctgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagacaattccaagaacacgctgtatctgcaaatgaacagcctgagagctgaggacacggccatttattactgtgcgagagtgatggggacggatccccactcctactactacatggacgtctgggggaaggggaccctggtcaccgtctcgagt

SEQ ID NO: 247 - protein, 123 aa, Artificial Sequence
  chemically synthesized
QVQLVESGGGVVRPGRSLRLSCAASGFTFSSYGMHWVRQAPGKGLEWVAVISYDGSIKYYADSVKGRFTISRDNSKNTLYLQMNSLRAEDTAIYYCARVMGTDPHSYYYMDVWGKGTLVTVSS

SEQ ID NO: 248 - protein, 14 aa, Artificial Sequence
  chemically synthesized
VMGTDPHSYYYMDV

SEQ ID NO: 249 - DNA, 354 bp, Artificial Sequence
  chemically synthesized
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagacaattccaagaacacgctgtatctgcaaatgaacagcctgagagctgaggacgcggctgtgtattactgtgcgagagcaccagatggccaccaacttgactactggggcaggggcaccctggtcaccgtctcgagt

SEQ ID NO: 250 - protein, 118 aa, Artificial Sequence
  chemically synthesized
QVQLVESGGGVVQPGRSLRLSCAASGFTFSSYGMHWVRQAPGKGLEWVAVISYDGSIKYYADSVKGRFTISRDNSKNTLYLQMNSLRAEDAAVYYCARAPDGHQLDYWGRGTLVTVSS

SEQ ID NO: 251 - DNA, 330 bp, Artificial Sequence
  chemically synthesized
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaagatgaccaaagaccctctgacgtccctgatcgcttctctggctccatcgacagctcctccaactctgcctccctcaccatctctggactgaggactgaggacgaggctgactactactgtcagtcttatgttagcagcaagtgggtgttcggcggagggaccaagctgaccgtccta

SEQ ID NO: 252 - protein, 110 aa, Artificial Sequence
  chemically synthesized
NFMLTQPHSVSESPGKTVTISCTGSSGSIASNYVQWYQQRPGSAPTTVIYEDDQRPSDVPDRFSGSIDSSSNSASLTISGLRTEDEADYYCQSYVSSKWVFGGGTKLTVL

SEQ ID NO: 253 - protein, 9 aa, Artificial Sequence
  chemically synthesized
APDGHQLDY

SEQ ID NO: 254 - protein, 7 aa, Artificial Sequence
  chemically synthesized
EDDQRPS

SEQ ID NO: 255 - protein, 9 aa, Artificial Sequence
  chemically synthesized
QSYVSSKWV

SEQ ID NO: 256 - DNA, 402 bp, Artificial Sequence
  chemically synthesized
caggtgcagctggtgcagtctggggctgaggtgaagaagcctgggtcctcggtgacggtctcctgcaaggcctctggaggcaccttcagcagcttttctatcacctggctgcgacaggcccctggacaagggcttgagtggatgggagagatcacccctatgtttggtatagcaaactacgcacagaagttccagggtagggtcacgattagcgcggacgagtccacgagcacagcctacatggagttgagtggcctgacatctgaagacacggccatgtattattgtgcgagagatggtcggtttgatgtttccgatcttttgactgacaaacccaaagtaacgataaactacaacgggatggacgtctggggccaaggcaccctggtcaccgtctcgagt

SEQ ID NO: 257 - protein, 134 aa, Artificial Sequence
  chemically synthesized
QVQLVQSGAEVKKPGSSVTVSCKASGGTFSSFSITWLRQAPGQGLEWMGEITPMFGIANYAQKFQGRVTISADESTSTAYMELSGLTSEDTAMYYCARDGRFDVSDLLTDKPKVTINYNGMDVWGQGTLVTVSS

SEQ ID NO: 258 - DNA, 330 bp, Artificial Sequence
  chemically synthesized
cagtctgtgctgactcagccaccctcagcgtctgggacccccgggcagagggtcaccatctcttgttctggaagcagctccaacatcgggagtaacactgtaaactggtaccagcgactcccaggagcggccccccaactcctcatctacaataatgaccagcggccctcagggatccctgaccgattctctggctccaagtctggcacctcaggctccctggtcatcagtgggctccagtctgaagatgaggctgattactactgtgcgtcatgggatgacagtctgaatggtcgggtgttcggcggagggaccaagctgaccgtccta

SEQ ID NO: 259 - protein, 110 aa, Artificial Sequence; identical to SEQ ID NO: 239
  chemically synthesized

SEQ ID NO: 260 - protein, 5 aa, Artificial Sequence
  chemically synthesized
SFSIT

SEQ ID NO: 261 - protein, 17 aa, Artificial Sequence
  chemically synthesized
EITPMFGIANYAQKFQG

SEQ ID NO: 262 - protein, 25 aa, Artificial Sequence
  chemically synthesized
DGRFDVSDLLTDKPKVTINYNGMDV

SEQ ID NO: 263 - DNA, 378 bp, Artificial Sequence
  chemically synthesized
cagctggtggagtctggaggaggcttgatccagcctggggggtccctgagactttcctgtgcagcctctggattcagcgtcagtgacacctacatgaactgggtccgccaggctccagggaagggcctggagtgggtgtcaagtatttatagcgatgatagcacatactacgcagactccgtgaagggcagattcaccatctccagagacaattccaagaacacgctgtttcttcaaataaacagtttgagagccgaggacacggctgtctattactgtgcgagagataaggagtatgtaacatcaactgggggcgcctactactacttctactacatggacgtctggggccagggcaccctggtcaccgtctcgagt

SEQ ID NO: 264 - protein, 126 aa, Artificial Sequence
  chemically synthesized
QLVESGGGLIQPGGSLRLSCAASGFSVSDTYMNWVRQAPGKGLEWVSSIYSDDSTYYADSVKGRFTISRDNSKNTLFLQINSLRAEDTAVYYCARDKEYVTSTGGAYYYFYYMDVWGQGTLVTVSS

SEQ ID NO: 265 - DNA, 330 bp, Artificial Sequence
  chemically synthesized
cagtctgtgctgactcagccaccctcagcgtctgggacccccgggcagagagtctccatctcctgttctggaagcagctccaacatcggaagtgatactgtgaactggtaccagcacctcccaggaacggcccccaaactcctcatctataataataatcagcggccctcaggggtccctgaccgattctctggctccaagtctggcacctcagcctccctggccatcagtgggctccagtctgaggatgaggctgattattactgtgccgcatgggatgacagcctgaatggtctggtattcggcggagggaccaaggtcaccgtccta

SEQ ID NO: 266 - protein, 110 aa, Artificial Sequence
  chemically synthesized
QSVLTQPPSASGTPGQRVSISCSGSSSNIGSDTVNWYQHLPGTAPKLLIYNNNQRPSGVPDRFSGSKSGTSASLAISGLQSEDEADYYCAAWDDSLNGLVFGGGTKVTVL

SEQ ID NO: 267 - protein, 13 aa, Artificial Sequence
  chemically synthesized
SGSSSNIGSDTVN

SEQ ID NO: 268 - protein, 7 aa, Artificial Sequence
  chemically synthesized
NNNQRPS

SEQ ID NO: 269 - protein, 11 aa, Artificial Sequence
  chemically synthesized
AAWDDSLNGLV

SEQ ID NO: 270 - DNA, 351 bp, Artificial Sequence
  chemically synthesized
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagacgagtttgatgcttttgatatctggggccgagggacaatggtcaccgtctcgagt

SEQ ID NO: 271 - protein, 117 aa, Artificial Sequence
  chemically synthesized
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDEFDAFDIWGRGTMVTVSS

SEQ ID NO: 272 - DNA, 324 bp, Artificial Sequence
  chemically synthesized
tcctatgtgctgactcagcccccctcagtgtcggtggccccaggaacgacggccaggattacctgtgggggaaacaatatcggaagtaggagtgtgcattggtaccagcagaagccaggccaggcccctctactggtcatctattatgatagtgaccggccctcagggatccctctgcgattctctggctccaactctggaaacacggccaccctgaccatcagtagggtcgaagccggggatgaggccgactattactgtcaggtgtgggatactagtagtggtcattatgtcttcggaactgggaccaaggtcaccgtccta

SEQ ID NO: 273 - protein, 108 aa, Artificial Sequence
  chemically synthesized
SYVLTQPPSVSVAPGTTARITCGGNNIGSRSVHWYQQKPGQAPLLVIYYDSDRPSGIPLRFSGSNSGNTATLTISRVEAGDEADYYCQVWDTSSGHYVFGTGTKVTVL

SEQ ID NO: 274 - protein, 8 aa, Artificial Sequence
  chemically synthesized
DEFDAFDI

SEQ ID NO: 275 - protein, 11 aa, Artificial Sequence
  chemically synthesized
GGNNIGSRSVH

SEQ ID NO: 276 - protein, 7 aa, Artificial Sequence
  chemically synthesized
YDSDRPS

SEQ ID NO: 277 - protein, 11 aa, Artificial Sequence
  chemically synthesized
QVWDTSSGHYV

SEQ ID NO: 278 - DNA, 354 bp, Artificial Sequence
  chemically synthesized
cagctggtggagtctgggggaggcgtggtccagcctgggaggtccctgagactctcctgtgcagcctctggattcaccttcagtacctatggcatgcactgggtccgccaggctccaggcaaggggctggagtgggtggcagttatatcatatgatggaggtactaaatactatgcagactccgtgaagggccgattcaccatctccagagacaattccatgaaaacgctctatctgcaaatgaacagcctgagaactgaggacacggctgtgtattactgtgcgaaagatctgggggacctacccccgggccttgactactggggccaggggacaatggtcaccgtctcgagt

SEQ ID NO: 279 - protein, 118 aa, Artificial Sequence
  chemically synthesized
QLVESGGGVVQPGRSLRLSCAASGFTFSTYGMHWVRQAPGKGLEWVAVISYDGGTKYYADSVKGRFTISRDNSMKTLYLQMNSLRTEDTAVYYCAKDLGDLPPGLDYWGQGTMVTVSS

SEQ ID NO: 280 - DNA, 333 bp, Artificial Sequence
  chemically synthesized
cagtctgtgctgactcagccaccctcagcgtctgggacccccgggcagagggtcaccatctcttgttctggaagcagctccaacatcggaagtaatactgtaaactggtaccagcagctcccaggagcggcccccaaactcctcatctatactaataatcagcggccctcaggggtccctgaccgattctctggctccaagtctggcacctcagcctccctggccatcagtgggctccagtctgaggatgaggctgattattactgtgcagcatgggatgacagcctgaatggtaatgtggtattcggcggagggaccaaggtcaccgtccta

SEQ ID NO: 281 - protein, 111 aa, Artificial Sequence
  chemically synthesized
QSVLTQPPSASGTPGQRVTISCSGSSSNIGSNTVNWYQQLPGAAPKLLIYTNNQRPSGVPDRFSGSKSGTSASLAISGLQSEDEADYYCAAWDDSLNGNVVFGGGTKVTVL

SEQ ID NO: 282 - protein, 5 aa, Artificial Sequence
  chemically synthesized
TYGMH

SEQ ID NO: 283 - protein, 17 aa, Artificial Sequence
  chemically synthesized
VISYDGGTKYYADSVKG

SEQ ID NO: 284 - protein, 11 aa, Artificial Sequence
  chemically synthesized
DLGDLPPGLDY

SEQ ID NO: 285 - protein, 7 aa, Artificial Sequence
  chemically synthesized
TNNQRPS

SEQ ID NO: 286 - protein, 12 aa, Artificial Sequence
  chemically synthesized
AAWDDSLNGNVV

SEQ ID NO: 287 - DNA, 351 bp, Artificial Sequence
  chemically synthesized
caggtgcagctggtggagtctgggggaggcgtggctcagtctgggaagtccctgagactctcctgtgcagcctctggattcaccttcagtaactatggcatgcactgggtccgccaggctccaggcaaggggctggagtggctggcagtcatatcatatgatggaagtaacagatactatgcagactccgtgaagggccgattcaccatctccagagacaattccaacaacacactgaatctgcaaatgagcagcctgagagctgaggacacggctctatattactgtgcgaaagatgccggggggccgcttgattactggggcaagggcaccctggtcaccgtctcgagt

SEQ ID NO: 288 - protein, 117 aa, Artificial Sequence
  chemically synthesized
QVQLVESGGGVAQSGKSLRLSCAASGFTFSNYGMHWVRQAPGKGLEWLAVISYDGSNRYYADSVKGRFTISRDNSNNTLNLQMSSLRAEDTALYYCAKDAGGPLDYWGKGTLVTVSS

SEQ ID NO: 289 - DNA, 337 bp, Artificial Sequence
  chemically synthesized
cagtctgtgctgactcagctgactcagccaccctcggtgtcactggccccaggacagacggccaccattacttgtgggggagacaacattggacgtaaaagtgtgcactggtaccagcagaagccaggccaggcccctgtgttggtcgtctatgatgacaccgaccggccctcagggatccctgagcgattctctggctccaactctgggaacacggccaccctaaccatcagcagggtcgaagccggggatgaggccgactattactgtcaggtgtgggatagtagtattgatcattcttgggtgttcggcggagggaccaagctgaccgtcctag

SEQ ID NO: 290 - protein, 112 aa, Artificial Sequence
  chemically synthesized
QSVLTQLTQPPSVSLAPGQTATITCGGDNIGRKSVHWYQQKPGQAPVLVVYDDTDRPSGIPERFSGSNSGNTATLTISRVEAGDEADYYCQVWDSSIDHSWVFGGGTKLTVL

SEQ ID NO: 291 - protein, 5 aa, Artificial Sequence
  chemically synthesized
NYGMH

SEQ ID NO: 292 - protein, 17 aa, Artificial Sequence
  chemically synthesized
VISYDGSNRYYADSVKG

SEQ ID NO: 293 - protein, 8 aa, Artificial Sequence
  chemically synthesized
DAGGPLDY

SEQ ID NO: 294 - protein, 11 aa, Artificial Sequence
  chemically synthesized
GGDNIGRKSVH

SEQ ID NO: 295 - protein, 7 aa, Artificial Sequence
  chemically synthesized
DDTDRPS

SEQ ID NO: 296 - protein, 12 aa, Artificial Sequence
  chemically synthesized
QVWDSSIDHSWV

SEQ ID NO: 297 - DNA, 363 bp, Artificial Sequence
  chemically synthesized
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagacaattccaggaacacgctgtatctgcagatgaacagcctgagacctgaggacacggctgtttattactgtgcgaaagattggggatttagcggctccctaacatttgattattggggccaagggacaatggtcaccgtctcgagt

SEQ ID NO: 298 - protein, 121 aa, Artificial Sequence
  chemically synthesized
QVQLVESGGGVVQPGRSLRLSCAASGFTFSSYGMHWVRQAPGKGLEWVAVISYDGSIKYYADSVKGRFTISRDNSRNTLYLQMNSLRPEDTAVYYCAKDWGFSGSLTFDYWGQGTMVTVSS

SEQ ID NO: 299 - DNA, 324 bp, Artificial Sequence
  chemically synthesized
tcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaaggtcaccgtccta

SEQ ID NO: 300 - protein, 108 aa, Artificial Sequence
  chemically synthesized
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDHVVFGGGTKVTVL

SEQ ID NO: 301 - protein, 12 aa, Artificial Sequence
  chemically synthesized
DWGFSGSLTFDY

SEQ ID NO: 302 - protein, 11 aa, Artificial Sequence
  chemically synthesized
GGNNIGSKSVH

SEQ ID NO: 303 - protein, 11 aa, Artificial Sequence
  chemically synthesized
QVWDSSSDHVV

SEQ ID NO: 304 - DNA, 351 bp, Artificial Sequence
  chemically synthesized
caggtgcagctggtggagtctgggggaggcgtggctcactctgggaagtccctgagactctcctgtgcagcctctggattcaccttcagtaactatggcatgcactgggtccgccaggctccaggcaaggggctggagtggctggcagtcatatcatatgatgggagtaatagatactatgcagactccgtgaagggccgattcaccatctccagagacaattccaacaacacgctgaatctgcaaatgagcagcctgagagctgaggacacggctctgtattactgtgcgaaagatgccggggggccgcttgattactggggccggggcaccctggtcaccgtctcgagt

SEQ ID NO: 305 - protein, 117 aa, Artificial Sequence
  chemically synthesized
QVQLVESGGGVAHSGKSLRLSCAASGFTFSNYGMHWVRQAPGKGLEWLAVISYDGSNRYYADSVKGRFTISRDNSNNTLNLQMSSLRAEDTALYYCAKDAGGPLDYWGRGTLVTVSS

SEQ ID NO: 306 - DNA, 330 bp, Artificial Sequence
  chemically synthesized
tcctatgtgctgactcagccaccctcggtgtcagtggccccaggacagatggccagaattacctgtgggggaaacaacattggagataaaagtgtgcaatggtaccagcagaggccaggccaggcccctctactggtcgtctatgatgatagcgaccggccctcagggatccctgagcgcttctctggctcctactctaggaacacggccaccctgaccatcagcagggtcgaagccggggatgaggccgactattactgtcaggtgtgggatagtagtagtgatcatccggaggtggttttcggcggagggaccaagctgaccgtccta

SEQ ID NO: 307 - protein, 110 aa, Artificial Sequence
  chemically synthesized
SYVLTQPPSVSVAPGQMARITCGGNNIGDKSVQWYQQRPGQAPLLVVYDDSDRPSGIPERFSGSYSRNTATLTISRVEAGDEADYYCQVWDSSSDHPEVVFGGGTKLTVL

SEQ ID NO: 308 - protein, 11 aa, Artificial Sequence
  chemically synthesized
GGNNIGDKSVQ

SEQ ID NO: 309 - protein, 7 aa, Artificial Sequence
  chemically synthesized
DDSDRPS

SEQ ID NO: 310 - protein, 13 aa, Artificial Sequence
  chemically synthesized
QVWDSSSDHPEVV

SEQ ID NO: 311 - DNA, 360 bp, Artificial Sequence
  chemically synthesized
caggtccagctggtgcagtctgggggaggcgtggtccagcctgggaggtccctgagactctcctgtgcagcctctggattcaccttcagtacctatggcatgcactgggtccgccaggctccaggcaaggggctggagtgggtggcagttatatcatatgatggaggtactaaatactatgcagactccgtgaagggccgattcaccatctccagagacaattccatgaaaacgctctatctgcaaatgaacagcctgagaactgaggacacggctgtgtattactgtgcgaaagatctgggggacctacccccgggccttgactactggggccgagggacaatggtcaccgtctcgagt

SEQ ID NO: 312 - protein, 120 aa, Artificial Sequence
  chemically synthesized
QVQLVQSGGGVVQPGRSLRLSCAASGFTFSTYGMHWVRQAPGKGLEWVAVISYDGGTKYYADSVKGRFTISRDNSMKTLYLQMNSLRTEDTAVYYCAKDLGDLPPGLDYWGRGTMVTVSS

SEQ ID NO: 313 - DNA, 321 bp, Artificial Sequence
  chemically synthesized
tcctatgagctgactcagccaccctcagtgtcagtggccctgggacagacggccacgattacctgtggggggagcagtattgagagtaaaagtgtacactggtaccaggagaagccaggccaggcccctgtcctggtcatctataaagattccaaccggccctctgtgatccctgagcgattctctggctccaactcggggaacacggccaccctgaccatcggcagagcccaagccggggatgaggctgactattactgtcaggtgtgggacagcagtactggtgtggtattcggcggagggaccaagctgaccgtccta

SEQ ID NO: 314 - protein, 128 aa, Artificial Sequence
  chemically synthesized
CHEMICALLYSYNTHESIZEDSYELTQPPSVSVALGQTATITCGGSSIESKSVHWYQEKPGQAPVLVIYKDSNRPSVIPERFSGSNSGNTATLTIGRAQAGDEADYYCQVWDSSTGVVFGGGTKLTVL

SEQ ID NO: 315 - protein, 11 aa, Artificial Sequence
  chemically synthesized
GGSSIESKSVH

SEQ ID NO: 316 - protein, 7 aa, Artificial Sequence
  chemically synthesized
KDSNRPS

SEQ ID NO: 317 - protein, 10 aa, Artificial Sequence
  chemically synthesized
QVWDSSTGVV

SEQ ID NO: 318 - DNA, 360 bp, Artificial Sequence
  chemically synthesized
caggtccagttggtggagtctgggggaggcgtggtccagcctgggaggtccctgagactctcctgtgcagcctctggattcaccttcagtacctatggcatgcactgggtccgccaggctccaggcaaggggctggagtgggtggcagttatatcatatgatggaggtactaaatactatgcagactccgtgaagggccgattcaccatctccagagacaattccatgaaaacgctctatctgcaaatgaacagcctgagaactgaggacacggctgtgtattactgtgcgaaagatctgggggacctacccccgggccttgactactggggccaggggacaatggtcaccgtctcgagt

SEQ ID NO: 319 - protein, 120 aa, Artificial Sequence
  chemically synthesized
QVQLVESGGGVVQPGRSLRLSCAASGFTFSTYGMHWVRQAPGKGLEWVAVISYDGGTKYYADSVKGRFTISRDNSMKTLYLQMNSLRTEDTAVYYCAKDLGDLPPGLDYWGQGTMVTVSS

SEQ ID NO: 320 - DNA, 333 bp, Artificial Sequence
  chemically synthesized
cagtctgtgctgactcagccaccctcagcgtctgggacccccgggcagagggtcaccatctcttgttctggaagcagctccaacatcggaagtaatactgtaaactggtaccagcagctcccaggagcggcccccaaactcctcatctatactaataatcagcggccctcaggggtccccgaccgattctctggctccaagtctggcacctcagcctccctggccatcagtgggctccagtctgaggatgaggctgattattactgtgcagcatgggatgacagctcggagcctcgtgtggtattcggcggagggaccaaggtcaccgtccta

SEQ ID NO: 321 - protein, 111 aa, Artificial Sequence
  chemically synthesized
QSVLTQPPSASGTPGQRVTISCSGSSSNIGSNTVNWYQQLPGAAPKLLIYTNNQRPSGVPDRFSGSKSGTSASLAISGLQSEDEADYYCAAWDDSSEPRVVFGGGTKVTVL

SEQ ID NO: 322 - protein, 12 aa, Artificial Sequence
  chemically synthesized
AAWDDSSEPRVV

SEQ ID NO: 323 - protein, 5 aa, Artificial Sequence
  chemically synthesized
DTYMN

SEQ ID NO: 324 - protein, 16 aa, Artificial Sequence
  chemically synthesized
SIYSDDSTYYADSVKG

SEQ ID NO: 325 - protein, 20 aa, Artificial Sequence
  chemically synthesized
DKEYVTSTGGAYYYFYYMDV

SEQ ID NO: 326 - protein, 10 aa, Artificial Sequence
  chemically synthesized
DGSESEYLDY

SEQ ID NO: 327 - protein, 13 aa, Artificial Sequence
  chemically synthesized
TGSSGSIASNYVQ

SEQ ID NO: 328 - protein, 217 aa, Artificial Sequence
  chemically synthesized
NFMLTQPHSVSESPGKTVTISCTGSGGSIASNYVQWYQQRPGSSPTTVIYEDNQRPSGVPDRFSGSIDSSSNSASLTISGLKTEDEADYYCQSYDPLPVWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 329 - protein, 449 aa, Artificial Sequence
  chemically synthesized
QVQLVESGGGVVQPGRSLRLSCAASGFTFSNSGIHWVRQAPGKGLEWVAVISYDGSNKYYADSVKGRFTISRDNSKNTLYLQMNSLRAEDTAVYYCARLRDNAEYTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 330 - DNA, 360 bp, Artificial Sequence
  chemically synthesized
gaggtgcagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagacggtggctggtacgactggtacttcgatctctggggcaggggaaccctggtcaccgtctcgagt

SEQ ID NO: 331 - protein, 120 aa, Artificial Sequence
  chemically synthesized
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDGGWYDWYFDLWGRGTLVTVSS

SEQ ID NO: 332 - DNA, 324 bp, Artificial Sequence
  chemically synthesized
tcttctgagctgactcaggaccctgatgtgtccgtggccttgggacagacagtcaggatcacatgccaaggagacagcctcaccagctattatgcaagctggtaccagcagaagccaggacaggcccctgtacttgtcatctctggtaatgacaaccggccctcagggatcccagaccgattctctggctccaactcaggaaacacagcttccttgaccatcactggggctcaggcggaagatgcggctgactattactgtggctcccgggacagcagcggttaccaagtggtgttcggcgcagggaccaagctgaccgtccta

SEQ ID NO: 333 - protein, 108 aa, Artificial Sequence
  chemically synthesized
SSELTQDPDVSVALGQTVRITCQGDSLTSYYASWYQQKPGQAPVLVISGNDNRPSGIPDRFSGSNSGNTASLTITGAQAEDAADYYCGSRDSSGYQVVFGAGTKLTVL

SEQ ID NO: 334 - protein, 11 aa, Artificial Sequence
  chemically synthesized
DGGWYDWYFDL

SEQ ID NO: 335 - protein, 11 aa, Artificial Sequence
  chemically synthesized
QGDSLTSYYAS

SEQ ID NO: 336 - protein, 7 aa, Artificial Sequence
  chemically synthesized
GNDNRPS

SEQ ID NO: 337 - protein, 11 aa, Artificial Sequence
  chemically synthesized
GSRDSSGYQVV

SEQ ID NO: 338 - DNA, 324 bp, Artificial Sequence
  chemically synthesized
tcttctgagctgactcaggaccctgctgtgtctgtggccttgggacagacagtcaggatcacatgccaaggagacagcctcagaagctattatgcaagctggtaccagcggaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtgcttttcggcggagggaccaagctgaccgtccta

SEQ ID NO: 339 - protein, 108 aa, Artificial Sequence
  chemically synthesized
SSELTQDPAVSVALGQTVRITCQGDSLRSYYASWYQRKPGQAPVLVIYGKNNRPSGIPDRFSGSSSGNTASLTITGAQAEDEADYYCNSRDSSGNHVLFGGGTKLTVL

SEQ ID NO: 340 - protein, 450 aa, Artificial Sequence
  5E3 heavy chain amino acid sequence
QIQLVQSGPELKKPGESVKISCKASGYTFTDYVMYWVKQAPGKGLKWMGWINTNTGKPTYADDFKGRFVFSLEASASTANLQISNLKNEDTATYFCTRGNYPGLRVMDAWGQGASVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 341 - protein, 120 aa, Artificial Sequence
  5E3 VH amino acid sequence
QIQLVQSGPELKKPGESVKISCKASGYTFTDYVMYWVKQAPGKGLKWMGWINTNTGKPTYADDFKGRFVFSLEASASTANLQISNLKNEDTATYFCTRGNYPGLRVMDAWGQGASVTVSS

SEQ ID NO: 342 - protein, 214 aa, Artificial Sequence
  5E3 light chain amino acid sequence
DIQMTQSPSSMSVSLGDTVTITCRASQDVGIYVNWFQQKPGKSPRRLIFRATNLADGVPSRFSGSRSGSDYSLTISSLGSEDVAGYHCLQYDEYPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 343 - protein, 107 aa, Artificial Sequence
  5E3 VL amino acid sequence
DIQMTQSPSSMSVSLGDTVTITCRASQDVGIYVNWFQQKPGKSPRRLIFRATNLADGVPSRFSGSRSGSDYSLTISSLGSEDVAGYHCLQYDEYPLTFGSGTKLEIK

SEQ ID NO: 344 - protein, 8 aa, Artificial Sequence
  5E3 CDRH1 amino acid sequence
GYTFTDYV

SEQ ID NO: 345 - protein, 8 aa, Artificial Sequence
  5E3 CDRH2 amino acid sequence
INTNTGKP

SEQ ID NO: 346 - protein, 13 aa, Artificial Sequence
  5E3 CDRH3 amino acid sequence
TRGNYPGLRVMDA

SEQ ID NO: 347 - protein, 6 aa, Artificial Sequence
  5E3 CDRL1 amino acid sequence
QDVGIY

SEQ ID NO: 348 - protein, 3 aa, Artificial Sequence
  5E3 CDRL2 amino acid sequence
RAT

SEQ ID NO: 349 - protein, 9 aa, Artificial Sequence
  5E3 CDRL3 amino acid sequence
LQYDEYPLT

SEQ ID NO: 350 - protein, 137 aa, Artificial Sequence
  NI-0101 VH amino acid sequence
MGWSWIFLFLLSGTAGVHCQVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDPSDAFPYWGQGTLVTVSS

SEQ ID NO: 351 - protein, 126 aa, Artificial Sequence
  NI-0101 VL amino acid sequence
MEWSWVFLFFLSVTTGVHSEIVLTQSPDFQSVTPKEKVTITCRASQSISDHLHWYQQKPDQSPKLLIKYASHAISGVPSRFSGSGSGTDFTLTINSLEAEDAATYYCQQGHSFPLTFGGGTKVEIK

(19) United States
(12) Patent Application Publication
(10) Pub. No.: US 2023/0192881 A1
(43) Pub. Date: Jun. 22, 2023

(54) COMPOSITIONS AND METHODS FOR TREATMENT OF ACUTE RESPIRATORY DISTRESS SYNDROME

(71) Applicant: Edesa Biotech Research Inc., Markham (CA)

(72) Inventors: Michael John BROOKS, Toronto (CA); Blair Richard George GORDON, Toronto (CA); Pardeep NIJHAWAN, Markham (CA)

(21) Appl. No.: 17/920,323

(22) PCT Filed: Apr. 20, 2021
(86) PCT No.: PCT/CA2021/050544

§ 371 Date: Oct. 20, 2022

Related U.S. Application Data
(60) Provisional application No. 63/081,662, filed on Sep. 22, 2020.
(60) Provisional application No. 63/012,783, filed on Apr. 20, 2020.
(60) Provisional application No. 63/012,786, filed on Apr. 20, 2020.

(51) Int. Cl. C07K 16/28; C07K 16/24; A61P 11/00

(52) U.S. Cl. CPC C07K 16/2896; A61P 11/00; C07K 16/248; C07K 2317/24; C07K 2317/524; C07K 2317/565

(57) ABSTRACT

This invention relates generally to monoclonal antibodies or antigen binding fragments thereof that specifically bind Toll-like Receptor 4 (TLR-4), to methods of using the anti-TLR4 antibodies to treat or prevent symptoms of Acute Respiratory Distress Syndrome (ARDS). This invention also relates to monoclonal antibodies or antigen binding fragments thereof that specifically bind to IP-10 and methods of using anti-IL-10 antibodies to treat or prevent symptoms of ARDS. The invention further provides routes of administrations and formulations for said methods.

### CROSS-REFERENCE TO RELATED APPLICATIONS

This application is a U.S. National Phase application, filed under U.S.C. § 371, of International Application No. PCT/CA2021/050544, filed Apr. 20, 2021, which claims the priority to, and benefit of, U.S. Provisional Application No. 63/012,783, filed on Apr. 20, 2020, U.S. Provisional Application No. 63/012,786, filed on Apr. 20, 2020 and U.S. Provisional Application No. 63/081,662, filed on Sep. 22, 2020. The contents of each of these applications are hereby incorporated by reference in their entireties.

### FIELD OF INVENTION

This invention relates to monoclonal antibodies (e.g., humanized antibodies) that bind to Toll Like Receptor 4 (TLR4)/ Myeloid differentiation protein-2 (MD-2) complex), methods of producing such antibodies and methods of using such antibodies in the treatment of acute respiratory distress syndrome (ARDS). This invention also relates to antibodies and antigen binding fragments thereof that bind to IP-10, methods of producing such antibodies and methods of using such antibodies in the treatment of ARDS.

### INCORPORATION OF SEQUENCE LISTING

The contents of the text file named “EDBI-001_N01US_SeqListing_ST25.txt”, which was created on Oct. 19, 2022and is 253 KB in size, are hereby incorporated by reference in their entirety.

### BACKGROUND OF INVENTION

Acute Respiratory Distress Syndrome (ARDS), the clinical correlate of severe acute lung injury (ALI) in humans, is an important cause of morbidity and mortality in critically ill patients (Ware, L. B., and M. A Matthay, 2000. N Engl J Med 342:1334-1349. Goss, C. H. et al. 2003. Crit Care Med 31: 1607-1611. Mendez, J. L. and RD. Hubmayr, 2005. Curr Opin Crit Care 11:29-36. Rubenfeld, G. D. et al 2005. N Engl J Med 353:1685-1693). ARDS is a complex clinical syndrome. Sepsis and infectious pneumonia (including influenza, and coronavirus infections), are leading causes of ALI/ARDS (Ware, L. B., and M. A Matthay, 2000. Goss, C. H. et al. 2003) and ARDS can be aggravated by ventilator-induced injury. ARDS-associated mortality remains high at 30-50% despite optimal ICU supportive care (Ware. L. B., and M. A Matthay, 2000. The Acute Respiratory Distress Syndrome Network, 2000. Matthay, M. A. et al. 2003. Slutsky, A. S. and L. D. Hudson, 2006). Current therapeutic treatments are limited and include broad management techniques such as mechanical ventilation and fluid management. There exists a need for development of new therapeutic treatments of ARDS.

Viral infections, including human and avian influenza viruses and coronaviruses, such as Middle East Respiratory Syndrome (MERS or MERS-CoV), severe acute respiratory syndrome (SARS or SARS-CoV) and novel coronavirus that causes coronavirus 2019 (COVID-19, 2019-nCoV or SARS-CoV-2) are threats to the human population. COVID-19, originating in Wuhan, China, presents a severe threat to the world population and is the source of the current world-wide pandemic. One life-threatening complication that can arise from COVID-19 infection is ARDS. In a current study, more than 40% of individuals that were hospitalized for severe and critical COVID-19 developed ARDS – and over 50% of those diagnosed died from the disease (Wu et. al., JAMA Intern Med. Mar. 13, 2020. doi:10.1001/jamainternmed.2020.0994).

Previous experience with SARS and MERS has revealed that ARDS occurs in patients despite a diminishing viral load, suggesting that antiviral therapy alone may be inadequate to prevent disease progression and even death caused by an exuberant host immune response. Experience with COVID-19 for the past year has suggested two predominant drivers of COVID-19 pathogenesis. During the early stages of the disease, SARS-CoV-2 replication and viral load are the main culprits for disease symptoms. However, as the disease progresses, an over-activated immune/inflammatory response due to the initial viral infection causes tissue damage, inflammation and hospitalization.

Current efforts are focused on containment and quarantine of infected individuals. Because of the serious effect of COVID-19 infections on a host, the widespread infection rate, as well as the worldwide pandemic outbreak of COVID-19, there exists a critical need for new treatments.

Accordingly, there exists a need for development of therapeutics and methods to treat, prevent and improve the survival of patients with ARDS, influenza infections or coronavirus infections, such as COVID-19.

### SUMMARY OF THE INVENTION

The present disclosure provides a method of treating, preventing or alleviating a symptom of acute respiratory distress syndrome (ARDS) in a subject in need thereof comprising administering to the subject a composition comprising an antibody that binds specifically to a Toll-like Receptor 4 (TLR4) and MD-2 complex.

In some embodiments, the antibody comprises a) a heavy chain variable region comprising a complementarity determining region 1 (CDRH1) comprising the amino acid sequence of SEQ ID NOs: 1, 20 or 28; a complementarity determining region 2 (CDRH2) comprising the amino acid sequence of SEQ ID NOs: 2, 21 or 29; and a complementarity determining region 3 (CDRH3) comprising the amino acid sequence of SEQ ID NOs: 3, 22, 30, 186 or 187; and b) a light chain variable region comprising a complementarity determining region 1 (CDRL1) comprising the amino acid sequence of SEQ ID NOs: 4, 24, 33; a complementarity determining region 2 (CDRL2) comprising the amino acid sequence of SEQ ID NOs: 5, 25 or 34; and a complementarity determining region 3 (CDRL3) comprising the amino acid sequence of SEQ ID NOs: 6, 17, 26, 35, 188, 189, 190 or 191.

In some embodiments, the antibody comprises a CDRH1 region comprising the amino acid sequence of SEQ ID NO: 1; a CDRH2 region comprising the amino acid sequence of SEQ ID NO: 2, a CDRH3 region comprising the amino acid sequence of SEQ ID NO: 3; a CDRL1 region comprising the amino acid sequence of SEQ ID NO: 4; a CDRL2 region comprising the amino acid sequence of SEQ ID NO: 5; and a CDRL3 region comprising an amino acid sequence of SEQ ID NO: 6.

In some embodiments, the antibody comprises a CDRH1 region comprising the amino acid sequence of SEQ ID NO: 1; a CDRH2 region comprising the amino acid sequence of SEQ ID NO: 2, a CDRH3 region comprising the amino acid sequence of SEQ ID NO: 3; a CDRL1 region comprising the amino acid sequence of SEQ ID NO: 4; a CDRL2 region comprising the amino acid sequence of SEQ ID NO: 5; and a CDRL3 region comprising an amino acid sequence of SEQ ID NO: 17.

In some embodiments, the antibody comprises a CDRH1 region comprising the amino acid sequence of SEQ ID NO: 20; a CDRH2 region comprising the amino acid sequence of SEQ ID NO: 21, a CDRH3 region comprising the amino acid sequence of SEQ ID NO: 22; a CDRL1 region comprising the amino acid sequence of SEQ ID NO: 24; a CDRL2 region comprising the amino acid sequence of SEQ ID NO: 25; and a CDRL3 region comprising an amino acid sequence of SEQ ID NO: 26.

In some embodiments, the antibody comprises a CDRH1 region comprising the amino acid sequence of SEQ ID NO: 28; a CDRH2 region comprising the amino acid sequence of SEQ ID NO: 29, a CDRH3 region comprising the amino acid sequence of SEQ ID NO: 30; a CDRL1 region comprising the amino acid sequence of SEQ ID NO: 33; a CDRL2 region comprising the amino acid sequence of SEQ ID NO: 34; and a CDRL3 region comprising an amino acid sequence of SEQ ID NO: 35.

In some embodiments, the antibody comprises a heavy chain variable region comprising the amino acid sequence of SEQ ID NOs: 350, 14, 15, 19, 27, 31, 36 or 38 and a light chain variable region comprising the amino acid sequence of SEQ ID NOs: 351, 16, 18, 23, 32, 40, 42, 44 or 46.

In some embodiments, the antibody comprises a heavy chain variable region comprising the amino acid sequence of SEQ ID NO: 350 and a light chain variable region comprising the amino acid sequence of SEQ ID NO: 351. In some embodiments, the antibody comprises a heavy chain variable region comprising the amino acid sequence of SEQ ID NO: 14 and a light chain variable region comprising the amino acid sequence of SEQ ID NO: 16. In some embodiments, the antibody comprises a heavy chain variable region comprising the amino acid sequence of SEQ ID NO: 14 and a light chain variable region comprising the amino acid sequence of SEQ ID NO: 18. In some embodiments, the antibody comprises a heavy chain variable region comprising the amino acid sequence of SEQ ID NO: 15 and a light chain variable region comprising the amino acid sequence of SEQ ID NO: 16. In some embodiments, the antibody comprises a heavy chain variable region comprising the amino acid sequence of SEQ ID NO: 15 and a light chain variable region comprising the amino acid sequence of SEQ ID NO: 18. In some embodiments, the antibody comprises a heavy chain variable region comprising the amino acid sequence of SEQ ID NO: 19 and a light chain variable region comprising the amino acid sequence of SEQ ID NO: 23. In some embodiments, the antibody comprises a heavy chain variable region comprising the amino acid sequence of SEQ ID NO: 27 and a light chain variable region comprising the amino acid sequence of SEQ ID NO: 32. In some embodiments, the antibody comprises a heavy chain variable region comprising the amino acid sequence of SEQ ID NO: 31 and a light chain variable region comprising the amino acid sequence of SEQ ID NO: 32. In some embodiments, the antibody comprises a heavy chain variable region comprising the amino acid sequence of SEQ ID NO: 36 and a light chain variable region comprising the amino acid sequence of SEQ ID NO: 40. In some embodiments, the antibody comprises a heavy chain variable region comprising the amino acid sequence of SEQ ID NO: 36 and a light chain variable region comprising the amino acid sequence of SEQ ID NO: 42. In some embodiments, the antibody comprises a heavy chain variable region comprising the amino acid sequence of SEQ ID NO: 36 and a light chain variable region comprising the amino acid sequence of SEQ ID NO: 44. In some embodiments, the antibody comprises a heavy chain variable region comprising the amino acid sequence of SEQ ID NO: 36 and a light chain variable region comprising the amino acid sequence of SEQ ID NO: 46. In some embodiments, the antibody comprises a heavy chain variable region comprising the amino acid sequence of SEQ ID NO: 38 and a light chain variable region comprising the amino acid sequence of SEQ ID NO: 40. In some embodiments, the said antibody comprises a heavy chain variable region comprising the amino acid sequence of SEQ ID NO: 38 and a light chain variable region comprising the amino acid sequence of SEQ ID NO: 42. In some embodiments, the antibody comprises a heavy chain variable region comprising the amino acid sequence of SEQ ID NO: 38 and a light chain variable region comprising the amino acid sequence of SEQ ID NO: 44. In some embodiments, the antibody comprises a heavy chain variable region comprising the amino acid sequence of SEQ ID NO: 38 and a light chain variable region comprising the amino acid sequence of SEQ ID NO: 46.

In some embodiments, the heavy chain amino acid sequence comprises the amino acid sequence of SEQ ID NO: 9 and the light chain amino acid sequence comprises the amino acid sequence of SEQ ID NO: 10.

In some embodiments, the antibody is a monoclonal antibody. In some embodiments, the antibody is a humanized antibody.

In some embodiments, the antibody is an IgG isotype. In some embodiments, the antibody is an IgGl isotype. In some embodiments, the amino acid residues at EU positions 325-328 of the CH2 domain of a IgG1 consist of an amino acid motif of SKAF (SEQ ID NO: 193).

In some embodiments, the antibody is administered by inhalation, nasally, intravenously, subcutaneously, intramuscularly or any combination thereof. In some embodiments, the antibody is administered at a dose of 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 35 mg/kg, about 40 mg/kg or about 50 mg/kg. In some embodiments, the antibody is administered at about 15 mg/kg.

In some embodiments, the antibody is administered at least one time, at least 2 times, at least 3 times, at least 4 times, at least 5 times, at least 6 times, at least 7 times, at least 8 times, at least 9 times or at least 10 times. In some embodiments, the antibody is administered once time.

In some embodiments, the antibody is administered one time, at a dose of about 15 mg/kg, intravenously.

In some embodiments, a symptom of ARDS is acute onset of bilateral alveolar infiltrates, hypoxemia, acute hypoxemia, lobar collapse, lung collapse, productive cough, fatigue, fever, chest pain, shortness of breath, labored breathing, increased heart rage, low blood pressure, confusion, extreme tiredness, respiratory failure, pulmonary vascular leak, pulmonary edema, alveolar epithelial cell injury, alveolar endothelial cell injury, alveolar capillary membrane barrier disruption or any combination thereof.

In some embodiments, the subject has a coronavirus infection, a viral infection, an influenza infection, sepsis, an aspiration pneumonitis, an infectious pneumonia, severe trauma, a fracture, a pulmonary contusion, an inhalation injury, a transfusion related injury, HSCT, pancreatitis, cytokine storm from cancer therapeutics, a collagen vascular disease, a drug effect from ingestants, a drug effect from inhalants, shock, acute eosinophilic pneumonia, immunologically mediated pulmonary hemorrhage and vasculitis, radiation pneumonitis or any combination thereof.

In some embodiments, the subject has a coronavirus infection. In some embodiments, the subject is suspected of having a coronavirus infection. In some embodiments, the subject has been exposed to a coronavirus or wherein the subject is suspected to have been exposed to a coronavirus; and the subject has not developed a symptom of a coronavirus infection. In some embodiments, the coronavirus is 229E alpha coronavirus, NL63 alpha coronavirus, OC43 beta coronavirus, HKU1 beta coronavirus, Middle East Respiratory Syndrome beta coronavirus (MERS-CoV or MERS), Severe Acute Respiratory Syndrome beta corona virus (SARS-CoV or SARS), a B1.1.7 variant of SARS-CoV, a B.1.351 variant of SARS-CoV, a P.1 variant of SARS-CoV, a B.1.427 variant of SARS-CoV, a B.1.4 variant of SARS-CoV, or novel coronavirus that causes coronavirus disease 2019 (SARS-CoV-2 or COVID-19).

In some embodiments, the symptom of the coronavirus infection is shortness of breath, dyspnea, dry cough, fever, runny nose, nasal congestion, anosmia, loss of smell, muscle aches, muscle pains, fatigue, respiratory sputum production, headache, vomiting, hemoptysis, sore throat, myalgia, diarrhea or any combination thereof.

In some embodiments, the method of the disclosure further comprises administering an antiviral drug, an ACE inhibitor, immune booster drug, a corticosteroid or any combination thereof. In some embodiments, the antiviral drug is Remdesivir, bamlanivimab, etesevimab, casirivimab, imdevimab or a monoclonal antibody targeting the virus. In some embodiments, the ACE inhibitor is hydroxychloroquine or a soluble recombinant ACE2. In some embodiments, the immune booster drug is an anti-IL6 antibody, an anti-IP-10 antibody, an anti IL-1 antibody or an anti-TNF antibody. In some embodiments, the IL6 antibody is tocilizumab or sarilumab. In some embodiments, the immune booster drug is atorvastatin or pravastatin. In some embodiments, the corticosteroid is dexamethasone.

The present disclosure also provides an injectable pharmaceutical formulation comprising about 10 mg/mL of an antibody that binds specifically to TLR4, about 1.88 mg/mL of L-Histidine, about 2.70 mg/mL of L-Histidine monohydrochloride, monohydrate, about 68.46 mg/mL of sucrose and about 0.05 mg/mL of polysorbate 80.

The present disclosure also provides a injectable pharmaceutical formulation comprising about 150 mg/mL of an antibody that binds specifically to TLR4, about 5.24 mg/mL of L-Histidine monohydrochloride, about 40.15 mg/mL of L-Arginine monohydrochloride, about 1.65 mg/mL of L-Arginine and about 0.20 mg/mL of polysorbate 80.

In some embodiments, the antibody that binds specifically to TLR4 comprises a CDRH1 region comprising the amino acid sequence of SEQ ID NO: 1; a CDRH2 region comprising the amino acid sequence of SEQ ID NO: 2, a CDRH3 region comprising the amino acid sequence of SEQ ID NO: 3; a CDRL1 region comprising the amino acid sequence of SEQ ID NO: 4; a CDRL2 region comprising the amino acid sequence of SEQ ID NO: 5; and a CDRL3 region comprising an amino acid sequence of SEQ ID NO: 6. In some embodiments, the antibody that binds specifically to TLR4 comprises a heavy chain variable region comprising the amino acid sequence of SEQ ID NO: 350 and a light chain variable region comprising the amino acid sequence of SEQ ID NO: 351.

The present disclosure provides a method of treating, preventing or alleviating a symptom of acute respiratory distress syndrome (ARDS) in a subject in need thereof comprising administering to the subject a composition comprising an antibody or antigen binding fragment thereof that binds to IP-10.

In some embodiments, the antibody comprises a) a heavy chain variable region comprising a complementarity determining region 1 (CDRH1) comprising the amino acid sequence of SEQ ID NO:201, 230, 240, 260, 282, 291, or 323; a complementarity determining region 2 (CDRH2) comprising the amino acid sequence of SEQ ID NO:202, 211, 231, 241, 261, 283, 292, or 324; and a complementarity determining region 3 (CDRH3) comprising the amino acid sequence of SEQ ID NO: 203, 217, 232, 242, 248, 253, 262, 274, 284, 293, 301, 325, 326 or 334; and b) a light chain variable region comprising a complementarity determining region 1 (CDRL1) comprising the amino acid sequence of SEQ ID NO: 204, 212, 233, 243, 267, 275, 294, 302, 308, 315, 327 or 335; a complementarity determining region 2 (CDRL2) comprising the amino acid sequence of SEQ ID NO: 205, 234, 244, 254, 268, 276, 285, 295, 309, 316 or 336; and

a complementarity determining region 3 (CDRL3) comprising SEQ ID NO: 206, 220, 235, 235, 245, 255, 269, 277, 286, 296, 303, 310, 317, 322 or 337.

In some embodiments, the antibody comprises a CDRH1 region comprising the amino acid sequence of SEQ ID NO: 201; a CDRH2 region comprising the amino acid sequence of SEQ ID NO: 202, a CDRH3 region comprising the amino acid sequence of SEQ ID NO: 203; a CDRL1 region comprising the amino acid sequence of SEQ ID NO: 204; a CDRL2 region comprising the amino acid sequence of SEQ ID NO: 205; and a CDRL3 region comprising an amino acid sequence of SEQ ID NO: 206.

In some embodiments, the antibody comprises a) a heavy chain variable region comprising an amino acid sequence of SEQ ID NOS: 198, 208, 214, 222, 227, 237, 247, 250, 257, 264, 271, 279, 288, 298, 305, 312, 319 or 331 and b) a light chain variable region comprising an amino acid sequence of SEQ ID NOS: 200, 210, 219, 224, 229, 239, 252, 259, 266, 273, 281, 290, 300, 307, 314, 321, 333 or 339.

In some embodiments, the antibody comprises a heavy chain variable region comprising the amino acid sequence of SEQ ID NO: 198 and a light chain variable region comprising the amino acid sequence of SEQ ID NO: 200.

In some embodiments, the antibody is a monoclonal antibody. In some embodiments, the antibody is a humanized antibody. In some embodiments, the antibody is an IgG isotype. In some embodiments, the antibody is an IgGl isotype. In some embodiments, the antibody is administered by inhalation, nasally, intravenously, subcutaneously, intramuscularly or any combination thereof.

In some embodiments, the antibody is administered at a dose of about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 35 mg/kg, about 40 mg/kg or about 50 mg/kg. In some embodiments, the antibody is administered at least one time, at least 2 times, at least 3 times, at least 4 times, at least 5 times, at least 6 times, at least 7 times, at least 8 times, at least 9 times or at least 10 times.

In some embodiments, a symptom of ARDS is acute onset of bilateral alveolar infiltrates, hypoxemia, acute hypoxemia, lobar collapse, lung collapse, productive cough, fatigue, fever, chest pain, shortness of breath, labored breathing, increased heart rage, low blood pressure, confusion, extreme tiredness, respiratory failure, pulmonary vascular leak, pulmonary edema, alveolar epithelial cell injury, alveolar endothelial cell injury, alveolar capillary membrane barrier disruption or any combination thereof.

In some embodiments, the subject has a coronavirus infection, a viral infection, an influenza infection, sepsis, an aspiration pneumonitis, an infectious pneumonia, severe trauma, a fracture, a pulmonary contusion, an inhalation injury, a transfusion related injury, HSCT, pancreatitis, cytokine storm from cancer therapeutics, a collagen vascular disease, a drug effect from ingestants, a drug effect from inhalants, shock, acute eosinophilic pneumonia, immunologically mediated pulmonary hemorrhage and vasculitis, radiation pneumonitis or any combination thereof.

In some embodiments, the subject has a coronavirus infection. In some embodiments, the subject is suspected of having a coronavirus infection. In some embodiments, the subject has been exposed to a coronavirus or wherein the subject is suspected to have been exposed to a coronavirus; and the subject has not developed a symptom of a coronavirus infection. In some embodiments, the coronavirus is 229E alpha coronavirus, NL63 alpha coronavirus, OC43 beta coronavirus, HKU1 beta coronavirus, Middle East Respiratory Syndrome beta coronavirus (MERS-CoV or MERS), Severe Acute Respiratory Syndrome beta corona virus (SARS-CoV or SARS), a B1.1.7 variant of SARS-CoV, a B.1.351 variant of SARS-CoV, a P.1 variant of SARS-CoV, a B.1.427 variant of SARS-CoV, a B.1.4 variant of SARS-CoV, or novel coronavirus that causes coronavirus disease 2019 (SARS-CoV-2 or COVID-19).

In some embodiments, the symptom of the coronavirus infection is shortness of breath, dyspnea, dry cough, fever, runny nose, nasal congestion, anosmia, loss of smell, muscle aches, muscle pains, fatigue, respiratory sputum production, headache, vomiting, hemoptysis, sore throat, myalgia, diarrhea or any combination thereof.

In some embodiments, the method of the disclosure further comprises administering an antiviral drug, an ACE inhibitor, immune booster drug, a corticosteroid or any combination thereof. In some embodiments, the antiviral drug is Remdesivir, bamlanivimab, etesevimab, casirivimab, imdevimab or a monoclonal antibody targeting the virus. In some embodiments, the ACE inhibitor is hydroxychloroquine or a soluble recombinant ACE2. In some embodiments, the immune booster drug is an anti-IL6 antibody, an anti-TLR4 antibody, an anti IL-1 antibody or an anti-TNF antibody. In some embodiments, the anti-TLR4 antibody is NI-0101. In some embodiments, the IL6 antibody is tocilizumab or sarilumab. In some embodiments, the immune booster drug is atorvastatin or pravastatin. In some embodiments, the corticosteroid is dexamethasone.

### BRIEF DESCRIPTION OF DRAWINGS

FIGS. 1A-1C shows that 5E3 inhibits LPS-induced cytokine storm in mouse blood in a dose dependent manner. CD1 mice were injected i.v. with 0.2, 0.04, 0.008, or 0.0016 mg/kg of 5E3 mAb or 0.2 mg/kg of isotype control mAb. 1 h later the mice were injected i.v. with 1 µg/kg of LPS. 2 h later the mice were euthanized and serum cytokines and chemokines were measured FIG. 1A shows the TNF-alpha concentration. FIG. 1B shows the IL-6 concentration. FIG. 1C shows the CCL5 concentration.

FIG. 2 shows a bar graph depicting inhibitory effect of 5E3 and 5E3 IgG2a D265A mutant on LPS-dependent neutrophil recruitment into the lung.

FIGS. 3A-3C shows that 5E3 inhibits LPS-induced IL-6, TNFalpha and CXCL10 secretion in the BAL fluid. 5E3 (black bars), 5E3 IgG₂ₐ D265A (blank bars) or isotype control (grey bar). BAL supernatants were separated from cells by centrifugation. Cytokines and chemokines were quantified. FIG. 3A shows IL-6 concentration in BAL. FIG. 3B shows TNFalpha concentration in BAL. FIG. 3C shows CXCL10 concentration in BAL.

FIG. 4 shows mean serum concentrations (µg/mL) of 5E3 in mice following intravenous administration of 5E3 (log/linear).

### DETAILED DESCRIPTION OF THE INVENTION

This invention relates to monoclonal antibodies (e.g., humanized antibodies or full human antibodies) or antigen binding fragments thereof that bind to Toll Like Receptor 4 (TLR4)/Myeloid differentiation protein-2 (MD-2) complex) and monoclonal antibodies that bind to interferon-inducible protein 10 (IP-10, CXCL10). The IP-10 antibodies are collectively referred to herein as huIP-10 antibodies. The TLR4 antibodies are collectively referred to herein as anti-TLR4 antibodies.

The present invention also provides methods of using these antibodies in the treatment of acute respiratory distress syndrome (ARDS). The present invention also provides methods of using these antibodies in the treatment, prevention or alleviation of a symptom of an influenza or coronavirus infection. Exemplary coronavirus infections include, but are not limited to SARS, MERS and COVID-19.

In some embodiments, the anti-TLR4 antibodies and antigen binding fragments thereof that bind TLR4. Anti-TLR4 antibodies include antibodies that bind the human TLR4/MD-2 receptor complex and also bind TLR4 independently of the presence of MD-2.

In some embodiments, the anti-TLR4 antibodies or antigen binding fragments thereof further comprise at least an FyR binding portion of an Fc region. In some embodiments, the anti-TLR4 antibodies or antigen binding fragments thereof include at least one specific amino acid substitution in the gamma heavy chain constant region such that the altered antibody elicits alterations in antigen-dependent effector function while retaining binding to antigen as compared to an unaltered antibody.

The altered antibodies of the invention include an altered antibody in which at least the amino acid residue at EU position 328 in the CH2 domain of the Fc portion of the antibody has been modified. For example, at least the amino acid residue at EU position 328 has been substituted with phenylalanine. In the altered antibodies described herein, at least the amino acid residue at EU position 328 alone or together with EU amino acid positions 325 and 326 are substituted with a different residue as compared to an unaltered antibody.

These altered antibodies with a modified Fc portion elicit modified effector functions e.g., a modified Fc receptor activity, as compared to an unaltered antibody. For example, the human Fc receptor is CD32A. In some embodiments, the altered antibodies elicit a prevention of proinflammatory mediators release following ligation to CD32A as compared to an unaltered antibody. Thus, the altered antibodies described herein elicit a modified Fc receptor activity, such as the prevention of proinflammatory mediators release while retaining the ability to bind a target antigen. In some embodiments, the altered antibody is a neutralizing antibody, wherein the altered antibody elicits a modified Fc receptor activity, while retaining the ability to neutralize one or more biological activities of a target antigen.

The anti-TLR4 antibodies of the invention inhibit receptor activation and subsequent intracellular signaling via LPS. The anti-TLR4 antibodies neutralize the activation of the TLR4/MD-2 receptor complex. These anti-TLR4 antibodies block LPS-induced and other TLR4 ligand-induced pro-inflammatory cytokine (e.g., IL-6, IL-8, TNFα) production.

### Acute Respiratory Syndrome

ARDS is a type of respiratory failure that is characterized by rapid onset of widespread inflammation in the lungs. The breakdown of the alveolar-capillary barrier leads to flooding of the alveolar space and fluid accumulation in the lungs. A Seattle case study described that 24 critically ill patients infected with SARS-CoV-2 who presented with acute hypoxemia were admitted to an ICU with poor prognosis and a 50% fatality rate. Furthermore, COVID-19 patients admitted to the ICU have shown secondary hemophagocytic lymphohistiocytosis (sHLH) and cytokine profiles which are characteristic of viral infections.

An overwhelming inflammatory response can cause ARDS and acute lung injury (ALI). Patients infected with COVID-19 can develop a surge of proinflammatory cytokines and inflammatory markers in what is known as a “cytokine storm” (CS). The pathology of CS is not fully understood. One of the questions about CS is why some individuals seem particularly susceptible, yet others seem relatively resistant. When viruses attack respiratory epithelial cells, pattern recognition receptors (PRR) of the immune cells recognize the virus and signal the production of cytokines like interferon gamma (IFN-y), tumor necrosis factors (TNFs), interleukins (ILs), and chemokines.

The acute respiratory distress syndrome (ARDS), the clinical correlate of severe acute lung injury (ALI) in humans, is an important cause of morbidity and mortality in critically ill patients (Ware, L. B., and M. A Matthay, 2000. N Engl J Med 342:1334-1349. Goss, C. H. et al. 2003. Crit Care Med 31: 1607-1611. Mendez, J. L. and RD. Hubmayr, 2005. Curr Opin Crit Care 11:29-36. Rubenfeld, G. D. et al 2005. N Engl J Med 353:1685-1693). Acute respiratory distress syndrome (ARDS) is an acute, diffuse inflammatory form of lung injury that is associated with a variety of etiologies. Etiologies and predisposing factors of ARDS include, but are not limited to those described in Table 1.

TABLE 1

Etiologies and Predisposing Factors of Acute Respiratory Syndrome

| | |
|---|---|
| Etiology | Clinical Features/Symptoms |
| Sepsis | Fever hypotension, leukocytosis, lactic acidosis, infectious source |
| Aspiration pneumonitis | Witnessed or risk for aspiration, food, lipid laden macrophages, airway erythema on cronchoscopy |
| Infectious pneumonia (including mycobacterial, viral, fungal, parasitic) | Productive cough, pleuritic pain, fever, leukocytosis, lobar consolidation or bilateral infiltrates in an immunosuppressed patient |
| Severe trauma and/or multiple fractures | History of trauma or fractures within the last week |
| Pulmonary contusion | History of chest trauma (blunt or penetrating), chest pain |
| Burns and smoke inhalation | Exposure to fire or smoke, cough, dyspnea, DIC, particulate matter on bronchoscopy, surface burns |
| Transfusion related acute lung injury and massive transfusions | History of transfusion, dyspnea during or shortly after transfusion |
| HSCT | History of HSCT |
| Pancreatitis | Abdominal pain, vomiting, risk factors (e.g., gall stones, alcohol, viral infection) |
| Inhalation injuries other than smoke (e.g. near drowning, gases) | History of inhalation exposure (e.g., chlorine gas) |
| Thoracic surgery (e.g. post-cardiopulmonary bypass) or other major surgery | History of surgery, intraoperative ventilation, intraoperative transfusion |
| Drugs (chemotherapeutic agents, amiodarone, radiation) | New drugs or radiation exposure in history, lymphocytosis on lavage, lavage may have suggestive features of amiodarone toxicity (“foamy macrophages”) but is non specific |
| Influenza | Fever, cough, sore throat, runny nose, stuffy nose, muscle aches, body aches, headaches, fatigue, vomiting, shortness of breath, persistent pressure in chest or abdomen |
| Coronavirus (MERS, SARS, COVID-19) | Shortness of breath, dyspnea, dry cough, fever, runny nose, nasal congestion, anosmia, loss of smell, muscle aches, muscle pains, sore throat, myalgia, diarrhea |

ARDS is characterized by various symptoms. Symptoms of ARDS include, but are not limited to acute onset of bilateral alveolar infiltrates, hypoxemia, shortness of breath, labored breathing, low blood pressure, confusion, extreme tiredness, respiratory failure, pulmonary vascular leak, pulmonary edema, alveolar epithelial cell injury, alveolar endothelial cell injury, alveolar capillary membrane barrier disruption or any combination thereof.

ARDS can be divided into three pathologic diagnosis and stages. 1) Early exudative stage – This stage occurs during the first 7-10 days is characterized by diffuse alveolar damage (DAD) is a non-specific reaction to lung injury from a variety of causes. Symptoms include, but are not limited to interstitial edema, acute and chronic inflammation, type II cell hyperplasia, hyaline membrane formation. 2) Fibroproliferative stage – After approximately 7-10 days, a proliferative stage develops. Symptoms include but are not limited to proliferation of type II alveolar cells, squamous metaplasia, interstitial infiltration by myofibroblasts, early deposition of collagen, pulmonary hypertension 3) Fibrotic stage – At this stage, symptoms include obliteration of normal lung architecture, fibrosis and cyst formation.

Histologically, ALI/ARDS in humans is characterized by a severe acute inflammatory response in the lungs and neutrophilic alveolitis (Ware. L. B., and M. A Matthay, 2000). Inflammatory stimuli from microbial pathogens, such as endotoxin (lipopolysaccharide, LPS), are well recognized for their ability to induce pulmonary inflammation, and experimental administration of LPS, both systemically and intratracheally, has been used to induce pulmonary inflammation in animal models of ALI (Kitamura. Y, S. et al. 2001. Am J Respir Crit Care Med 163:762-769. Matute-Bello, G. et al. 2004. Clin Diagn Lab Immunol 11:358-361. Rojas, M. et al. 2005. Am J Physiol Lung Cell Mol Physiol 288: L333-341. Altemeier, W. A. et al. 2005. J Immunol 175:3369-3376. Gharib, S. A, et al. 2006. Am J Respir Crit Care Med 173:653-658).

The physiological hallmark of ARDS is disruption of the alveolar-capillary membrane barrier (i.e., pulmonary vascular leak), leading to development of non-cardiogenic pulmonary edema in which a proteinaceous exudate floods the alveolar spaces, impairs gas exchange, and precipitates respiratory failure (Ware, L. B., and M. A Matthay, 2000. Ware, L. B., and M. A Matthay, 2001. Am J Respir Crit. Care Med 163: 1376-1383. Guidot, D. M. et al. 2006. Am J Physiol Lung Cell Mol Physiol 291:L301-306). Both alveolar epithelial and endothelial cell injury and/or death have been implicated in the pathogenesis of ALI/ARDS (Ware, L. B., and M. A Matthay, 2000). However, despite decades of research, few therapeutic strategies for clinical ARDS have emerged and current specific options for treatment are limited (Crimi, E., and A S. Slutsky, 2004. Best Pract Res Clin Anaesthesiol 18:477-492. The Acute Respiratory Distress Syndrome Network, 2000. N Engl J Med 342: 1301-1308. Matthay. M. A, et al. 2003. Am J Respir Crit Care Med 167: 1027-1035. Mehta, D. J. Bhattacharya. M. A Matthay, and A B. Malik, 2004. Am J Physiol Lung Cell Mol Physiol 287:L1081-1090. Slutsky, A S., and L. D. Hudson, 2006. N Engl J Med 354: 1839-1841). ARDS continues to be an important contributor to prolonged mechanical ventilation in the intensive care unit (ICU), and ARDS-associated mortality remains high at 30-50% despite optimal ICU supportive care (Ware. L. B., and M. A Matthay, 2000. The Acute Respiratory Distress Syndrome Network, 2000. Matthay, M. A. et al. 2003. Slutsky, A. S. and L. D. Hudson, 2006).

ARDS is a complex clinical syndrome which is initiated by injury to the lung, often in the setting of pneumonia and/or sepsis, and aggravated by ventilator-induced injury. Some of the early features of ARDS can be reproduced by administration of bacterial endotoxin (LPS), which acts via Toll-like receptor 4 (TLR4), to increase the expression of inflammatory cytokines and chemokines, and upregulate leukocyte adhesion molecules, results in EC activation (Kitamura, Y, S. et al. 2001. Matute-Bello. G. et al. 2004. Rojas, M. et al. 2005. Altemeier. W. A. et al. 2005. Gharib. S. A, et al. 2006. Fan, J, et al. J Clin Invest 112: 1234-1243). Accordingly, the use of an anti-TLR4 antibody or antigen binding fragment thereof to neutralize or antagonize the TLR4 signaling pathway may be useful in treatment of ARDS.

### Coronavirus

Coronavirus disease 2019 (COVID-19) is an infectious respiratory disease caused by the RNA beta-coronavirus Severe Acute Respiratory Syndrome Coronavirus 2 (SARS-CoV-2), which has a phylogenetic similarity to SARS-CoV. The virus was first identified in late December 2019, when it was discovered that several patients with viral pneumonia were epidemiologically related to the Huanan seafood market in Wuhan, China. By 11 Mar. 2020, the virus presence expanded beyond Chinese territory, reaching countries on all continents. Subsequently, the World Health Organization (WHO) declared COVID-19 as a pandemic. As of Apr. 5, 2021, COVID- 19 has affected 221 countries and territories, reporting over 132,211,138 confirmed cases, resulting in more than 2,869,491 deaths.

The signs and symptoms of COVID-19 vary, however, throughout the disease most persons will experience the following: Fever (83-99%), cough (59-82%), fatigue (44-70%), anorexia (40-84%), shortness of breath (31-40%), sputum production (28-33%) and myalgias (11-35%). Headache, confusion, rhinorrhea, sore throat, hemoptysis, and vomiting have been reported but are less frequent (<10%). Therefore, COVID-19 presents clinical manifestations that can range from mild to critical severity. Mild cases can typically resolve at home, whereas moderate or severe COVID-19 patients are hospitalized for observation and supportive care. Older age is associated with a higher risk of developing acute respiratory distress syndrome (ARDS) (hazard ratio [HR], 3.26; 95% CI 2.08-5.11). Other comorbidities that can increase the risk of developing severe or critical disease include cardiovascular disease (HR, 21.4; 95% 4.64 - 98.76), diabetes (HR, 2.38; 95% 1.35-4.05), hypertension (HR, 1.82; 95% 1.13-2.95), chronic lung disease (HR, 5.40; 95% CI 0.96-30.40) and obesity (7, 8).

Viral infections, including human and avian influenza viruses and coronavirus infections are serious threats to the human population. In humans, coronaviruses can cause respiratory tract infections that can range from mild to lethal. There are four main sub-groupings of coronaviruses, known as alpha, beta, gamma, and delta. Coronaviruses belong to the RNA virus belonging to the family Coronaviridae. Coronaviruses that can infect humans include, but are not limited to 29E alpha coronavirus, NL63 alpha coronavirus, OC43 beta coronavirus, HKU1 beta coronavirus, Middle East Respiratory Syndrome beta coronavirus (MERS-CoV or MERS), Severe Acute Respiratory Syndrome beta corona virus (SARS-CoV or SARS), or novel coronavirus that causes coronavirus disease 2019 (COVID-19, 2019-nCoV or SARS-CoV-2), or a mutant and/or variant thereof. Variants of SARS-CoV-2 include but are not limited to B.1.1.7, B. 1.351, P.1, B.1.427 or B.1.429. It is to be understood that new variants of coronavirus with novel mutations or sets of mutations can arise.

Symptoms of COVID-19 coronavirus infection include, but are not limited to shortness of breath, dyspnea, dry cough, fever, runny nose, nasal congestion, anosmia, loss of smell, muscle aches, muscle pains, fatigue, respiratory sputum production, headache, vomiting, hemoptysis, sore throat, myalgia, diarrhea or any combination thereof. Further development of the disease can lead to acute respiratory distress syndrome (ARDS), severe pneumonia, sepsis, septic shock and death.

The mechanism of COVID-19 pathogenesis remains unknown. However, IP-10 (CXCL10) has been shown to be overexpressed in critical patients with viral infections (Wang et. al., Cell Research (2013) 23:577-580. doi:10.1038/cr.2013.25). Furthermore, it is suggested that there are mild or severe cytokine storms in severe patients, which is also an important cause of death. Accordingly, treatment of cytokine storm may be an important part of reducing severity of symptoms. Excessive production of pro-inflammatory cytokines such as IL-6 and TNF-α are the major factors contributing to cytokine release syndrome (CRS) to progression of disease, lung tissue damage and eventually respiratory failures. Thus, anti-TLR4 antibodies which block LPS-induced and other TLR4 ligand-induced pro-inflammatory cytokine (e.g., IL-6, IL-8, TNFα) production may be useful in the treatment of COVID-19 infection.

### TLR4 Antibodies

Toll-like Receptors (TLR) are a type of PRRs that are part of a broad range of sensors that include nucleotide oligomerization domain (NOD)-like receptors (NLRs), retinoic acid-inducible gene (RIG)- like receptors (RLRs) and DNA receptors. Of the ten types of TLRs present in humans, TLR4 is the most extensively studied. TLR4 is expressed in endothelia, intestinal and lung epithelial, mast cells, neutrophils, and macrophages. Excessive activation of the TLR4- mediated inflammatory process has been linked to various inflammatory conditions. Prior to COVID-19, overactivation of TLR4 signaling by host-derived damage-associated molecular pattern molecules (DAMPs), such as oxidized phospholipids (Ox-PL), had been shown to be a key driver of viral-mediated acute lung injury. Patient cohort studies further support that TLR4 plays a prominent role in COVID-19 pathogenesis. The evidence appears to suggest that a proinflammatory cascade is initiated when viral-mediated cell damage occurs in the lungs, resulting in the release of DAMP molecules like S100A8/A9 (Calprotectin), HMGB1, resistin, and Ox-PL. These DAMPs are potent activators of TLR4 found on macrophages, neutrophils, dendritic cells and other innate immune cells and results in the release of pro-inflammatory molecules. Multiple patient cohort studies have recently shown that both HMGB1 and S100A8/A9 serum levels in hospitalized COVID-19 patients positively correlate with both neutrophil count, disease severity and importantly as an early predictor of disease progression. In particular, S100A8/A9 seems to have a higher degree of correlation with poor outcomes even at initial hospitalization than CRP or D-dimer and, S100A8/A9 levels were an incredibly a strong predictor of death.

Recent studies show that SARS-CoV-2 Spike protein directly binds to TLR4 with an affinity level typically observed for virus-receptor interactions. In addition, binding of the Spike protein to TLR4 stimulated the production of pro-inflammatory cytokines, like IL-1β, and the induction of these cytokines could be blocked by a small molecule inhibitor of TLR4.

It has been previously demonstrated that therapeutic antagonism of TLR4 signaling could protect against influenza-induced ALI. In mice, it was shown that administration of a TLR4 antagonist blocked influenza-induced lethality, as well as lung pathology, clinical symptoms, cytokine, and Ox-PL expression and decreased viral titers. Furthermore, antagonism of TLR4 can help modulate the secretion of proinflammatory cytokines (IL-6, IL8, and MIP-1β) by monocytes and dendritic cells. In mice, therapeutic blockade of TLR4 signaling offered protection from influenza- virus lethality and reduced cytokine gene expression in the lungs, protecting from ALI. Furthermore, ablation of the TLR4-S100A8/A9 interaction with the S100A8/A9 inhibitor paquinimod rescued humanized ACE2 mice from SARS-COV-2 infection.

Distinct monocyte and neutrophil populations seem to exist in severe COVID-19 patients and S100A8/A9 is thought to have a role in perpetuating the positive inflammatory feedback loop present in these patients. Furthermore, it has been shown that severe COVID-19 patients have higher levels of neutrophils in the lungs as compared to mild and healthy controls . In fact, neutrophil infiltration has been shown to be a hallmark of Acute Lung Injury (ALI)/ARDS in general.

TLR4 antibodies of the invention include, for example, antibodies having the combination of heavy chain and light chain sequences shown below.

Exemplary antibodies of the invention include, for example, the anti-TLR4 antibodies described in PCT/IB2005/004206, filed Jun. 14, 2005 and published as WO 2007/110678, the anti-TLR4 antibodies described in PCT application PCT/IB2008/003978, filed May 14, 2008 and published as WO 2009/101479, the contents of each of which are hereby incorporated by reference in their entirety, and commercially available antibodies such as HTA125.

Exemplary antibodies of the invention include, for example, the antibody referred to herein as NI-0101, which binds the human TLR4/MD2 complex and also binds TLR4 independently of the presence of MD-2. The sequences of the NI-0101 (hu15c1) antibody are shown below, with the CDR sequences underlined in the VH and VL amino acid sequences:

NI-0101 heavy chain nucleotide sequence:
ATGGGATGGAGCTGGATCTTTCTCTTCCTCCTGTCAGGAACTGCAGGTGT
ACATTGCCAGGTGCAGCTTCAGGAGTCCGGCCCAGGACTGGTGAAGCCTT
CGGACACCCTGTCCCTCACCTGCGCTGTCTCTGGTTACTCCATCACCGGT
GGTTATAGCTGGCACTGGATACGGCAGCCCCCAGGGAAGGGACTGGAGTG
GATGGGGTATATCCACTACAGTGGTTACACTGACTTCAACCCCTCCCTCA
AGACTCGAATCACCATATCACGTGACACGTCCAAGAACCAGTTCTCCCTG
AAGCTGAGCTCTGTGACCGCTGTGGACACTGCAGTGTATTACTGTGCGAG
AAAAGATCCGTCCGACGCCTTTCCTTACTGGGGCCAAGGGACTCTGGTCA
CTGTCTCTTCCGCCTCCACCAAGGGCCCATCGGTCTTCCCCCTGGCACCC
TCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGA
CCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGAC
CTACATCTGCAACGTGAATCACAAGCCCAGCAACACCAAGGTGGACAAGA
GAGTTGAGCCCAAATCTTGTGACAAAACTCACACATGCCCACCGTGCCCA
GCACCTGAACTCCTGGGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAATGCAAGGTCTCCAGTAAAGCTTTCCCTGCC
CCCATCGAGAAAACCATCTCCAAAGCCAAAGGGCAGCCCCGAGAACCACA
GGTGTACACCCTGCCCCCATCCCGGGAGGAGATGACCAAGAACCAGGTCA
GCCTGACCTGCCTGGTCAAAGGCTTCTATCCCAGCGACATCGCCGTGGAG
TGGGAGAGCAATGGGCAGCCGGAGAACAACTACAAGACCACGCCTCCCGT
GCTGGACTCCGACGGCTCCTTCTTCCTCTATAGCAAGCTCACCGTGGACA
AGAGCAGGTGGCAGCAGGGGAACGTCTTCTCATGCTCCGTGATGCATGAG
GCTCTGCACAACCACTACACGCAGAAGAGCCTCTCCCTGTCTCCGGGTAA
ATAG (SEQ ID NO: 12)

NI-0101 heavy chain amino acid sequence:
MGWSWIFLFLLSGTAGVHCQVQLQESGPGLVKPSDTLSLTCAVSGYSITG GYSWH WIRQPPGKGLEWMGYIHYSGYTDFNPSLKT RITISRDTSKNQFSL
KLSSVTAVDTAVYYCARKDPSDAFPY WGQGTLVTVSSASTKGPSVFPLA
PSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSSKAFP
APIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMH
EALHNHYTQKSLSLSPGK (SEQ ID NO: 9)

NI-0101 VH amino acid sequence:
MGWSWIFLFLLSGTAGVHCQVQLQESGPGLVKPSDTLSLTCAVSGYSITG GYSWH WIRQPPGKGLEWMGYIHYSGYTDFNPSLKT RITISRDTSKNQFSL
KLSSVTAVDTAVYYCARKDPSDAFPY WGQGTLVTVSS (SEQ ID NO:
350)

NI-0101 light chain nucleotide sequence:
ATGGAATGGAGCTGGGTCTTTCTCTTCTTCCTGTCAGTAACTACAGGTGT
CCACTCCGAAATTGTGTTGACGCAGTCTCCAGACTTTCAGTCTGTGACTC
CAAAGGAAAAAGTCACCATCACCTGCAGGGCCAGTCAGAGTATCAGCGAC
CACTTACACTGGTACCAACAGAAACCTGATCAGTCTCCCAAGCTCCTCAT
CAAATATGCTTCCCATGCCATTTCTGGGGTCCCATCGAGGTTCAGTGGCA
GTGGGTCTGGGACAGACTTCACTCTCACCATCAATAGCCTAGAGGCTGAA
GATGCTGCAACGTATTACTGTCAGCAGGGTCACAGTTTTCCGCTCACTTT
CGGCGGAGGGACCAAGGTGGAGATCAAACGTACGGTGGCTGCACCATCTG
TCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAG
AGCAGGACAGCAAGGACAGCACCTACAGCCTCAGCAGCACCCTGACGCTG
AGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTT
AG (SEQ ID NO: 13)

NI-0101 light chain amino acid sequence:
MEWSWVFLFFLSVTTGVHSEIVLTQSPDFQSVTPKEKVTITCRASQSISD HLH WYQQKPDQSPKLLIKYASHAIS GVPSRFSGSGSGTDFTLTINSLEAE
DAATYYCQQGHSFPLT FGGGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC (SEQ ID NO: 10)

NI-0101 VL amino acid sequence:
MEWSWVFLFFLSVTTGVHSEIVLTQSPDFQSVTPKEKVTITCRASQSISD HLH WYQQKPDQSPKLLIKYASHAIS GVPSRFSGSGSGTDFTLTINSLEAE
DAATYYCQQGHSFPLT FGGGTKVEIK (SEQ ID NO: 351)

The NI-0101 (hu15c1) antibody includes VH CDRs having the sequences GGYSWH (SEQ ID NO: 1), YIHYSGYTDFNPSLKT (SEQ ID NO: 2), and KDPSDAFPY (SEQ ID NO: 3), and VL CDRs having the sequences RASQSISDHLH (SEQ ID NO: 4), YASHAIS (SEQ ID NO: 5) and QQGHSFPLT (SEQ ID NO: 6).

The amino acid and nucleic acid sequences of the heavy chain variable (VH) and light chain variable (VL) regions of the anti-TLR4/MD-2 antibodies are shown below. In some embodiments, the amino acids encompassing the complementarity determining regions (CDR) as defined by Chothia et al. 1989, E.A. Kabat et al., 1991 are highlighted in underlined and italicized text below. (See Chothia, C, et al., Nature 342:877-883 (1989); Kabat, EA, et al., Sequences of Protein of immunological interest, Fifth Edition, US Department of Health and Human Services, US Government Printing Office (1991)). In some embodiments, exemplary antibodies of the invention include a combination of heavy chain and complementarity determining regions and light chain complementarity determining regions (CDRs) selected from the CDR sequences defined according to the IMGT nomenclature (See IMGT®, the international ImMunoGeneTics Information System®. Available online: http://www.imgt.org/).

Anti-TLR4 antibodies include the antibodies described in co-pending U.S. Applications 11/009939, filed Dec. 10, 2004 and 11/151916, filed Jun. 15, 2004 and in WO 05/065015, filed Dec. 10, 2004 and PCT/US2005/020930, filed Jun. 15, 2004, each of which is hereby incorporated by reference in its entirety. Several exemplary antibodies include the antibodies referred to therein as 18H10, 16G7, 15C1 and 7E3.

Anti-TLR4 antibodies include the antibodies described in co-pending U.S. Application 11/151916, filed Jun. 15, 2004 (U.S. Pat. Publication No. US 2008-0050366 A1) and in PCT/IB2005/004206, filed Jun. 15, 2004 (PCT Publication No. WO 07/110678), each of which is hereby incorporated by reference in its entirety. The sequences of several exemplary antibodies are shown below.

15Cl Hu V_{H} version 4-28
QVQLQESGPG LVKPSDTLSL TCAVSGYSI X₁ GGYSWHWIRQ PPGK
GLEW X₂G YIHYSGYTDF NPSLKTRX₃T X₄ SRDTSKNQFS LKLSS
VTAVD TAVYYCARKD PSDGFPYWGQ GTLVTVSS (SEQ ID NO: 1
4)

CDR 1: GGYSWH (SEQ ID NO: 1)

CDR 2: YIHYSGYTDFNPSLKT (SEQ ID NO: 2)

CDR 3: KDPSDGFPY (SEQ ID NO: 3)

Where X_{1 is} Thr or Ser
Where X₂ is Ile or Met
Where X₃ is Val or Ile
Where X₄ is Met or Ile

15C1 Hu V_{H} version 3-66
EVQLVESGGG LVQPGGSLRL SCAX₁SGYSIT GGYSWHWVRQ APGKG
LEWX₂S YIHYSGYTDFNPSLKTRFTI SRDNSKNTX₃Y LQMNSLRAED
TAVYYCARKD PSDGFPYWGQ GTLVTVSS (SEQ ID NO: 15)

CDR 1: GGYSWH (SEQ ID NO: 1)

CDR 2: YIHYSGYTDFNPSLKT (SEQ ID NO: 2)

CDR 3: KDPSDGFPY (SEQ ID NO: 3)

Where X₁ is Ala or Val
Where X₂ is Val or Met
Where X₃ is Leu or Phe

15C1 Hu VL version L6
EIVLTQSPAT LSLSPGERAT LSCRASQSIS DHLHWYQQKP GQAPRL
LIX₁Y ASHAISGIPA RFSGSGSGTD FTLTISSLEP EDFAVYYCQN
GHSFPLTFGG GTKVEIK (SEQ ID NO: 16)

CDR1: RASQSISDHLH (SEQ ID NO: 4)

CDR2: YASHAIS (SEQ ID NO: 5)

CDR3: QNGHSFPLT (SEQ ID NO: 17)

Where X₁ is Lys or Tyr

15C1 Hu VL version A26
EIVLTQSPDF QSVTPKEKVT ITCRASQSIS DHLHWYQQKP DQSPKLL
IKY ASHAISGVPS RFSGSGSGTD FTLTINSLEA EDAATYYCQN GH
SFPLTFGG GTKVEIK (SEQ ID NO: 18)

CDR1: RASQSISDHLH (SEQ ID NO: 4)

CDR2: YASHAIS (SEQ ID NO: 5)

CDR3: QNGHSFPLT (SEQ ID NO: 17)

18H10 Hu VH version 1-69 QVQLVQSGAE VKKPGSSVKV SCKASGFNIK DSYIHWVRQA PGQGLE
WX₁GW TDPENVNSIY DPRFQGRVTI TADX₂STSTAY X₃ELSSLRSE
D TAVYYCARGY NGVYYAMDYW GQGTTVTVSS (SEQ ID NO: 19)

CDR1: DSYIH (SEQ ID NO: 20)

CDR2: WTDPENVNSIYDPRFQG (SEQ ID NO: 21)

CDR3: GYNGVYYAMDY (SEQ ID NO: 22)

Where X₁ is Met or Ile
Where X₂ is Lys or Thr
Where X₃ is Met or Leu

18H10 Hu VL version L6
EIVLTQSPAT LSLSPGERAT LSCSASSSVI YMHWYQQKPG QAPRLL
IYRT YNLASGIPAR FSGSGSGTDX₁ TLTISSLEPE DFAVYYCHQW
SSFPYTFGQG TKVEIK (SEQ ID NO: 23)

CDR1: SASSSVIYMH (SEQ ID NO: 24)

CDR2: RTYNLAS (SEQ ID NO: 25)

CDR3: HQWSSFPYT (SEQ ID NO: 26)

Where X₁ is Phe or Tyr

7E3 Hu VH version 2-70
QVTLRESGPA LVKPTQTLTL TCTFSGFSLX₁ TYNIGVGWIR QPPGK
ALEWL AHIWWNDNIYYNTVLKSRLT X₂SKDTSKNQV VLTMTNMDPV
DTATYYCX₃RM AEGRYDAMDY WGQGTLVTVS S (SEQ
ID NO: 27)

CDR1: TYNIGVG (SEQ ID NO: 28)

CDR2: HIWWNDNIYYNTVLKS (SEQ ID NO: 29)

CDR3: MAEGRYDAMDY (SEQ ID NO: 30)

Where X₁ is Ser or Thr
Where X₂ is Ile or Phe
Where X₃ is Ile or Ala

7E3 Hu VH version 3-66
EVQLVESGGG LVQPGGSLRL SCAX₁SGFSLT TYNIGVGWVR QAPGK
GLEWX₂ SHIWWNDNIY YNTVLKSRLT X₃SX₄DNSKNTX₅ YLQMNSL
RAE DTAVYYCX₆RM AEGRYDAMDY WGQGTLVTVS S (SEQ ID NO:
31)

CDR1: TYNIGVG (SEQ ID NO: 28)

CDR2: HIWWNDNIYYNTVLKS (SEQ ID NO: 29)

CDR3: MAEGRYDAMDY (SEQ ID NO: 30)

Where X₁ is Phe or Ala
Where X₂ is Val or Leu
Where X₃ is Ile or Phe
Where X₄ is Lys or Arg
Where X₅ is Leu or Val
Where X₆ is Ile or Ala

7E3 Hu VL version L19
DIQMTQSPSS VSASVGDRVT ITCRASQDIT NYLNWYQQKP GKAPKL
LIYY TSKLHSGVPS RFSGSGSGTDX₁TLTISSLQP ED
FATYX₂CQQ GHSFPLTFGG GTKVEIK (SEQ ID NO: 32)

CDR1: RASQDITNYLN (SEQ ID NO: 33)

CDR2: YTSKLHS (SEQ ID NO: 34)

CDR3: QQGNTFPWT (SEQ ID NO: 35)

Where X₁ is Phe or Tyr
Where X₂ is Tyr or Phe

Anti-TLR4 antibodies include the antibodies described in PCT/IB2008/003978, filed May 14, 2008 (PCT Publication No. WO 2009/101479), the contents of which are hereby incorporated by reference in their entirety. These anti-TLR4 antibodies are modified to include one or more mutations in the CDR3 portion. The sequences of several exemplary antibodies are shown below.

15C1 humanized VH mutant 1 amino acid sequence:
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD PSDAFPY WGQGTLVTVSS (SEQ ID NO: 36)

15C1 humanized VH mutant 1 nucleic acid sequence:
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
CCGTCCGACGCCTTTCCTTACTGGGGCCAAGGGACTCTGGTCACTGTCTC
TTCC (SEQ ID NO: 37)

15C1 humanized VH mutant 2 amino acid sequence:
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD PSEGFPY WGQGTLVTVSS (SEQ ID NO: 38)

15C1 humanized VH mutant 2 nucleic acid sequence:
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
CCGTCCGAGGGATTTCCTTACTGGGGCCAAGGGACTCTGGTCACTGTCTC
TTCC (SEQ ID NO: 39)

15C1 humanized VL mutant 1 amino acid sequence:
EIVLTQSPDFQSVTPKEKVTITCRASQSISDHLHWYQQKPDQSPKLLIKY
ASHAISGVPSRFSGSGSGTDFTLTINSLEAEDAATYYCQNSHSFPLT FGG
GTKVEIK (SEQ ID NO: 40)

15C1 humanized VL mutant 1 nucleic acid sequence:
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGAATAGTCACAGTTTTCCGCTCACTTTCGGCGGA
GGGACCAAGGTGGAGATCAAA (SEQ ID NO: 41)

15C1 humanized VL mutant 2 amino acid sequence:
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLT FGG
GTKVEIK (SEQ ID NO: 42)

15C1 humanized VL mutant 2 nucleic acid sequence:
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 43)

15C1 humanized VL mutant 3 amino acid sequence:
EIVLTQSPDFQSVTPKEKVTITCRASQSISDHLHWYQQKPDQSPKLLIKY
ASHAISGVPSRFSGSGSGTDFTLTINSLEAEDAATYYCQNSSSFPLT FGG
GTKVEIK (SEQ ID NO: 44)

15C1 humanized VL mutant 3 nucleic acid sequence:
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGAATAGTAGTAGTTTTCCGCTCACTTTCGGCGGA
GGGACCAAGGTGGAGATCAAA (SEQ ID NO: 45)

15C1 humanized VL mutant 4 amino acid sequence:
EIVLTQSPDFQSVTPKEKVTITCRASQSISDHLHWYQQKPDQSPKLLIKY
ASHAISGVPSRFSGSGSGTDFTLTINSLEAEDAATYYCQQSHSFPLT FGG
GTKVEIK (SEQ ID NO: 46)

15C1 humanized VL mutant 4 nucleic acid sequence:
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGAGTCACAGTTTTCCGCTCACTTTCGGCGGA
GGGACCAAGGTGGAGATCAAA (SEQ ID NO: 47)

The variant CDR3 regions include VH CDR3- KDPSDAFPY (SEQ ID NO: 186); VH CDR3 - KDPSEGFPY (SEQ ID NO: 187); VL CDR3 - QNSHSFPLT (SEQ ID NO: 188); VL CDR3 - QQGHSFPLT (SEQ ID NO: 189); VL CDR3 - QNSSSFPLT (SEQ ID NO: 190); and VL CDR3 - QQSHSFPLT (SEQ ID NO: 191).

Anti-TLR4 antibodies include the antibodies described in PCT/IB2008/003978, filed May 14, 2008 (PCT Publication No. WO 2009/101479), the contents of which are hereby incorporated by reference in their entirety. These anti-TLR4 antibodies are modified to include one or more mutations in the CH2 domain. The sequences of several exemplary antibodies are shown below.

The invention also provides isolated polypeptides that include a gamma 1 Fc (γ1Fc) region, wherein amino acid residues at EU positions 325-328 of a CH2 domain. Table 2 shows exemplary mutations of the EU positions 325-328 of the CH2 domain of an IgG1. The invention also provides isolated polypeptides that include a gamma 1 Fc (γ1Fc) region, wherein amino acid residues at EU positions 325-328 of the CH2 domain of a IgG1 consist of an amino acid motif selected from SAAF (SEQ ID NO: 192), SKAF (SEQ ID NO: 193), NAAF (SEQ ID NO: 194) and NKAF (SEQ ID NO: 195).

TABLE 2

Amino acid sequences of the 6 mutants (C to H) at EU positions 325 to 328 within the CH2 domain of chimeric IgG1 15C1. Human residues are in bold

| chimeric IgG1 15C1 mutants | mutations from human to mouse amino acid residue at defined EU positions | number of mouse residues left overall in human CH2 | amino acid residues at EU positions 325 to 328 | SEQ ID NO: |
|---|---|---|---|---|
| human | | | NKAL | 196 |
| #H | L 328 F | 1 | NKAF | 195 |
| #E | K 326 A | 1 | NAAL | 197 |
| # D | N 325 S; K 326 A | 2 | SAAL | 198 |
| # G | K 326 A; L 328 F | 2 | NSAF | 194 |
| #F | N 325 S; L 328 F | 2 | SKAF | 193 |
| # C | N 325 S; K 326 A | 3 | SAAF | 192 |
| Mouse | | | SAAF | 192 |

In some embodiments, the altered antibodies include both a variant Fc region and a variant CDR3 region. In some embodiments, the altered antibodies include both a variant Fc region shown and a variant CDR3 region (SEQ ID NOs: 186-191). In some embodiments, the altered antibodies include both a variant CH2 domain in the Fc region and a variant CDR3 region. In some embodiments, the altered antibodies include both a variant CH2 domain in the Fc region shown in Table 2 and a variant CDR3 region e.g., SEQ ID NOs: 186-191. In some embodiments, the altered antibodies include both a variant CH2 domain in the Fc region that is mutated at one or more of the residues that correspond to residues 325, 326 and/or 328 (using the numbering of the residues in the gamma heavy chain as in the EU index, Edelman, et al.) and a variant CDR3 region. In some embodiments, the altered antibodies include both a variant CH2 domain in the Fc region that is mutated at one or more of the residues that correspond to residues 325, 326 and/or 328 (using the numbering of the residues in the gamma heavy chain as in the EU index, Edelman, et al.) and a variant CDR3 region SEQ ID NOs: 186-191.

The invention also provides methods of targeting human CD32A by a monoclonal antibody in which at least EU amino acid position 328 of the gamma heavy chain constant region together with one or two of the amino acid residues that correspond to EU positions 325 and 326 of the heavy gamma chain constant region are substituted with the corresponding EU amino acid residue of mouse IgG1 at the same position which is different from the corresponding amino acid residue in an unaltered antibody, such that the antibody elicits increased inhibition of pro-inflammatory mediators release upon binding to human CD32A while retaining binding to antigen as compared to an unaltered antibody. In some embodiments, the altered antibody further includes variant VH CDR3- KDPSDAFPY (SEQ ID NO: 186); VH CDR3 - KDPSEGFPY (SEQ ID NO: 187); VL CDR3 - QNSHSFPLT (SEQ ID NO: 188); VL CDR3 - QQGHSFPLT (SEQ ID NO: 189); VL CDR3 -QNSSSFPLT (SEQ ID NO: 190); and VL CDR3 - QQSHSFPLT (SEQ ID NO: 191).

In some embodiments, the amino acid residue that corresponds to EU position 325 of the gamma heavy chain constant region is substituted with serine. In some embodiments, the amino acid residue that corresponds to EU position 326 of gamma heavy chain constant region is substituted with alanine. In some embodiments, the amino acid residue that corresponds to EU position 328 of the gamma heavy chain constant region is substituted with phenylalanine. In some embodiments, the altered antibody further includes variant VH CDR3- KDPSDAFPY (SEQ ID NO: 186); VH CDR3 - KDPSEGFPY (SEQ ID NO: 187); VL CDR3 - QNSHSFPLT (SEQ ID NO: 188); VL CDR3 - QQGHSFPLT (SEQ ID NO: 189); VL CDR3 - QNSSSFPLT (SEQ ID NO: 190); and VL CDR3 - QQSHSFPLT (SEQ ID NO: 191).

Antibodies of the invention interfere with or otherwise antagonize signaling via human TLR4 and/or human TLR4/MD-2 complexes. In some embodiments, antibodies of the invention also bind cynomolgus monkey TLR4 and/or cynomolgus monkey TLR4/MD-2 complexes. In some embodiments, the antibody binds to an epitope that includes one or more amino acid residues on human and/or cynomolgus monkey TLR4 having the following sequences:

>Human TLR4 amino acid sequence
MMSASRLAGTLIPAMAFLSCVRPESWEPCVEVVPNITYQCMELNFYKIPD
NLPFSTKNLDLSFNPLRHLGSYSFFSFPELQVLDLSRCEIQTIEDGAYQS
LSHLSTLILTGNPIQSLALGAFSGLSSLQKLVAVETNLASLENFPIGHLK
TLKELNVAHNLIQSFKLPEYFSNLTNLEHLDLSSNKIQSIYCTDLRVLHQ
MPLLNLSLDLSLNPMNFIQPGAFKEIRLHKLTLRNNFDSLNVMKTCIQGL
AGLEVHRLVLGEFRNEGNLEKFDKSALEGLCNLTIEEFRLAYLDYYLDDI
IDLFNCLTNVSSFSLVSVTIERVKDFSYNFGWQHLELVNCKFGQFPTLKL
KSLKRLTFTSNKGGNAFSEVDLPSLEFLDLSRNGLSFKGCCSQSDFGTTS
LKYLDLSFNGVITMSSNFLGLEQLEHLDFQHSNLKQMSEFSVFLSLRNLI
YLDISHTHTRVAFNGIFNGLSSLEVLKMAGNSFQENFLPDIFTELRNLTF
LDLSQCQLEQLSPTAFNSLSSLQVLNMSHNNFFSLDTFPYKCLNSLQVLD
YSLNHIMTSKKQELQHFPSSLAFLNLTQNDFACTCEHQSFLQWIKDQRQL
LVEVERMECATPSDKQGMPVLSLNITCQMNKTIIGVSVLSVLVVSVVAVL
VYKFYFHLMLLAGCIKYGRGENIYDAFVIYSSQDEDWVRNELVKNLEEGV
PPFQLCLHYRDFIPGVAIAANIIHEGFHKSRKVIVVVSQHFIQSRWCIFE
YEIAQTWQFLSSRAGIIFIVLQKVEKTLLRQQVELYRLLSRNTYLEWEDS
VLGRHIFWRRLRKALLDGKSWNPEGTVGTGCNWQEATSI (SEQ ID NO
: 11)

>Cynomolgus monkey TLR4 amino acid sequence 1
MTSALRLAGTLIPAMAFLSCVRPESWEPCVEVVPNITYQCMELKFYKIPD
NIPFSTKNLDLSFNPLRHLGSYSFLRFPELQVLDLSRCEIQTIEDGAYQS
LSHLSTLILTGNPIQSLALGAFSGLSSLQKLVAVETNLASLENFPIGHLK
TLKELNVAHNLIQSFKLPEYFSNLTNLEHLDLSSNKIQNIYCKDLQVLHQ
MPLSNLSLDLSLNPINFIQPGAFKEIRLHKLTLRSNFDDLNVMKTCIQGL
AGLEVHRLVLGEFRNERNLEEFDKSSLEGLCNLTIEEFRLTYLDCYLDNI
IDLFNCLANVSSFSLVSVNIKRVEDFSYNFRWQHLELVNCKFEQFPTLEL
KSLKRLTFTANKGGNAFSEVDLPSLEFLDLSRNGLSFKGCCSQSDFGTTS
LKYLDLSFNDVITMSSNFLGLEQLEHLDFQHSNLKQMSQFSVFLSLRNLI
YLDISHTHTRVAFNGIFDGLLSLKVLKMAGNSFQENFLPDIFTDLKNLTF
LDLSQCQLEQLSPTAFDTLNKLQVLNMSHNNFFSLDTFPYKCLPSLQVLD
YSLNHIMTSNNQELQHFPSSLAFLNLTQNDFACTCEHQSFLQWIKDQRQL
LVEAERMECATPSDKQGMPVLSLNITCQMNKTIIGVSVFSVLVVSVVAVL
VYKFYFHLMLLAGCIKYGRGENIYDAFVIYSSQDEDWVRNELVKNLEEGV
PPFQLCLHYRDFIPGVAIAANIIHEGFHKSRKVIVVVSQHFIQSRWCIFE
YEIAQTWQFLSSRAGIIFIVLQKVEKTLLRQQVELYRLLSRNTYLEWEDS
VLGQHIFWRRLRKALLDGKSWNPEEQ (SEQ ID NO: 77)

Antibodies of the invention interfere with or otherwise antagonize signaling via human and/or cynomolgus monkey TLR4 and/or human and/or cynomolgus monkey TLR4/MD-2 complexes. In some embodiments, the antibody binds to an epitope that includes one or more amino acid residues on human and/or cynomolgus monkey TLR4 between residues 289 and 375 of SEQ ID NO: 11-(human TLR4) and/or SEQ ID NO: 77 (cynomolgus TLR4). For example, TLR4 antibodies specifically bind to an epitope that includes residue 349 of SEQ ID NO: 11 (human) and/or SEQ ID NO: 77 (cynomolgus). In some embodiments, the epitope also includes additional residues, for example, residues selected from the group consisting of at least residues 328 and 329 of SEQ ID NO: 11 (human) and/or SEQ ID NO: 77 (cynomolgus); at least residue 351 of SEQ ID NO: 11 (human) and/or SEQ ID NO: 77 (cynomolgus); and at least residues 369 through 371 of SEQ ID NO: 11 (human) and/or SEQ ID NO: 77 (cynomolgus), and any combination thereof.

In some embodiments, the anti-TLR4 antibody or immunologically active fragment thereof is or is derived from monoclonal antibodies recognizing human and/or cynomolgus monkey TLR4/MD-2 receptor expressed on the cell surface. The antibodies are capable of blocking, e.g., neutralizing, receptor activation and subsequent intracellular signaling induced TLR4 ligands, e.g., LPS or any other TLR4 ligand described herein. Antibodies of the invention include antibodies that bind human and cynomolgus monkey TLR4/MD-2 receptor complex and also bind TLR4 independently of the presence of MD-2.

In some embodiments, the anti-TLR4 antibody or immunologically active fragment thereof interferes with or otherwise antagonizes signaling via human and/or cynomolgus monkey TLR4/MD-2 receptor expressed on the cell surface, e.g., by blocking receptor activation and subsequent intracellular signaling induced by LPS. Exemplary monoclonal antibodies of these embodiments include: 1A1, 1A6, 1B12, 1C7, 1C10, 1C12, 1D10, 1E11, 1E11 N103D, 1G12, 1E11.C1, 1E11.C2, 1E11.C3, 1E11.C4, 1E11.C5, 1E11.C6, 1E11.E1, 1E11.E2, 1E11.E3, 1E11.E4, 1E11.E5, 1E11.C2E1, 1E11.C2E3, 1E11.C2E4 and 1E11.C2E5. The sequences of these antibodies are shown below.

These antibodies have distinct specificities. Some antibodies show specificity for both the human and cynomolgus monkey TLR4 and/or both the human and cynomolgus monkey TLR4/MD-2 receptor complex, and they have been shown to inhibit receptor activation and subsequent intracellular signaling via LPS. For example, 1C12, 1E11, 1E11 N103D, 1E11.C1, 1E11.C2, 1E11.C3, 1E11.C4, 1E11.C5, 1E11.C6, 1E11.C2E1, 1E11.C2E2, 1E11.C2E3, 1E11.C2E4 and 1E11.C2E5 bind both human and cynomolgus monkey TLR4 independently of the presence of human or cynomolgus monkey MD-2. 1A1, 1A6, 1B12, 1C7, 1C10, 1D10 and 1G12 only bind to cynomolgus monkey TLR4 independently of the presence of cynomolgus monkey MD-2. 1E11.E1, 1E11.E2, 1E11.E3, 1E11.E4 and 1E11.E5 bind only to human TLR4 independently of the presence of human MD-2.

In some embodiments, the invention provides an isolated antibody that specifically binds Toll-like receptor 4 (TLR4), wherein the antibody binds to an epitope that includes at least residue 349 of SEQ ID NO: 11 and an epitope that includes at least residue 349 of SEQ ID NO: 77. In some embodiments, the antibody includes a heavy chain with three complementarity determining regions (CDRs) including a variable heavy chain complementarity determining region 1 (CDRH1) amino acid sequence of GYSITGGYS (SEQ ID NO: 49); a variable heavy chain complementarity determining region 2 (CDRH2) amino acid sequence of IHYSGYT (SEQ ID NO: 56); and a variable heavy chain complementarity determining region 3 (CDRH3) amino acid sequence of ARKDSG(X₁)(X₂)(X₃)PY (SEQ ID NO: 57), where Xi is N, Q, D or E, X₂ is any hydrophobic amino acid, and X₃ is any hydrophobic amino acid; and a light chain with three CDRs including a variable light chain complementarity determining region 1 (CDRL1) amino acid sequence of QSISDH (SEQ ID NO: 68); a variable light chain complementarity determining region 2 (CDRL2) amino acid sequence of YAS (SEQ ID NO: 69); and a variable light chain complementarity determining region 3 (CDRL3) amino acid sequence of QQGHSFPLT (SEQ ID NO: 6). In some embodiments, the epitope further includes at least residues 328 and 329 of SEQ ID NO: 11 and SEQ ID NO: 77. In some embodiments, the epitope further includes at least residue 351 of SEQ ID NO: 11 and SEQ ID NO: 77. In some embodiments, the epitope further includes one or more residues between residues 369 through 371 of SEQ ID NO: 11 and SEQ ID NO: 77. In some embodiments, the epitope further includes at least residues 369 through 371 of SEQ ID NO: 11 and SEQ ID NO: 77. In some embodiments, the antibody specifically binds to an epitope that includes at least residues 328, 329, 349, 351 and 369 through 371 of SEQ ID NO: 11 and SEQ ID NO: 77. In some embodiments, the antibody further includes an amino acid substitution in the gamma heavy chain constant region at EU amino acid position 325 and an amino acid substitution at EU amino acid position 328. In some embodiments, the amino acid substituted at EU amino acid position 325 is serine, and wherein the amino acid substituted at EU amino acid position 328 is phenylalanine.

In some embodiments, the three heavy chain CDRs include an amino acid sequence at least 90%, 91%, 92%, 93%, 94%, 95%, 96%, 97% 98%, 99% or more identical to a variable heavy chain complementarity determining region 1 (VH CDR1, also referred to herein as CDRH1) amino acid sequence selected from the group consisting of G(F/Y)PI(R/G/W)(Y/F/G)GYS (SEQ ID NO: 48), GYSITGGYS (SEQ ID NO: 49); GFPIRYGYS (SEQ ID NO: 50); GYPIRFGYS (SEQ ID NO: 51); GYPIRHGYS (SEQ ID NO: 52); GFPIGQGYS (SEQ ID NO: 53); GYPIWGGYS (SEQ ID NO: 54) and GYPIGGGYS (SEQ ID NO: 55), a variable heavy chain complementarity determining region 2 (VH CDR2, also referred to herein as CDRH2) amino acid sequence of IHYSGYT (SEQ ID NO: 56); and a variable heavy chain complementarity determining region 3 (VH CDR3, also referred to herein as CDRH3) amino acid sequence selected from the group consisting of ARKDSG(N/Q/D/E)X₁X₂PY (SEQ ID NO: 57) where X₁ and X₂ are each independently any hydrophobic amino acid, ARKDSGNYFPY (SEQ ID NO: 58); ARKDSGRLLPY (SEQ ID NO: 59); ARKDSGKWLPY (SEQ ID NO: 60); ARKDSGHLMPY (SEQ ID NO: 61); ARKDSGHNYPY (SEQ ID NO: 62); ARKDSGKNFPY (SEQ ID NO: 63); ARKDSGQLFPY (SEQ ID NO: 64); ARKDSGHNLPY (SEQ ID NO: 65); ARKDSGDYFPY (SEQ ID NO: 66) and ARKDSGRYWPY (SEQ ID NO: 67). The three light chain CDRs include an amino acid sequence at least 90%, 91%, 92%, 93%, 94%, 95%, 96%, 97% 98%, 99% or more identical to a variable light chain complementarity determining region 1 (VL CDR1, also referred to herein as CDRL1) amino acid sequence of QSISDH (SEQ ID NO: 68); a variable light chain complementarity determining region 2 (VL CDR2, also referred to herein as CDRL2) amino acid sequence of YAS (SEQ ID NO: 69); and a variable light chain complementarity determining region 3 (VL CDR3, also referred to herein as CDRL3) amino acid sequence selected from the group consisting of QQG(Y/N)(D/E)(F/Y)PXT (SEQ ID NO: 70) where X is any hydrophobic amino acid, QQGHSFPLT (SEQ ID NO: 6); QQGNDFPVT (SEQ ID NO: 71); QQGYDEPFT (SEQ ID NO: 72); QQGYDFPLT (SEQ ID NO: 73); QQGYDYPLT (SEQ ID NO: 74) and QQGYEFPLT (SEQ ID NO: 75). The antibodies bind to human and cynomolgus monkey TLR4/MD-2 complex, to human and cynomolgus TLR4 when not complexed with human and cynomolgus MD-2, to human TLR4/MD-2 complex, to human TLR4 when not complexed with human MD-2, to cynomolgus monkey TLR4/MD-2 complex or cynomolgus TLR4 when not complexed with cynomolgus MD-2.

An exemplary TLR4 monoclonal antibody is the 1E11 antibody described herein. As shown below, the 1E11 antibody includes a heavy chain variable region (SEQ ID NO: 79) encoded by the nucleic acid sequence shown in SEQ ID NO: 78, and a light chain variable region (SEQ ID NO: 81) encoded by the nucleic acid sequence shown in SEQ ID NO: 80.

>1E11 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 78)

>1E11 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 79)

>1E11 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 80)

>1E11 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 81)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11 antibody have the following sequences: GYSITGGYS (SEQ ID NO: 49); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1A1 antibody described herein.

>1A1 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCCGGCCGCCTCCTCCCTTACTGGGGCCAAGGGACTCTGGTCACTGTCTC
TTCC (SEQ ID NO: 82)

>1A1 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGRLLPYWGQGTLVTVSS (SEQ ID NO: 83)

>1A1 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 84)

>1A1 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 85)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1A1 antibody have the following sequences: GYSITGGYS (SEQ ID NO: 49); IHYSGYT (SEQ ID NO: 56); and ARKDSGRLLPY (SEQ ID NO: 59). The light chain CDRs of the 1A1 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1A6 antibody described herein.

>1A6 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCAAGTGGTTGCCTTACTGGGGCCAAGGGACTCTGGTCACTGTCTC
TTCC (SEQ ID NO: 86)

>1A6 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGKWLPYWGQGTLVTVSS (SEQ ID NO: 87)

>1A6 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 88)

>1A6 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 89)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1A6 antibody have the following sequences: GYSITGGYS (SEQ ID NO: 49); IHYSGYT (SEQ ID NO: 56); and ARKDSGKWLPY (SEQ ID NO: 60). The light chain CDRs of the 1A6 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1B12 antibody described herein.

>1B12 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGGCACCTCATGCCTTACTGGGGCCAAGGGACTCTGGTCACTGTCTC
TTCC (SEQ ID NO: 90)

>1B12 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGHLMPYWGQGTLVTVSS (SEQ ID NO: 91)

>1B12 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 92)

>1B12 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 93)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1A6 antibody have the following sequences: GYSITGGYS (SEQ ID NO: 49); IHYSGYT (SEQ ID NO: 56); and ARKDSGHLMPY (SEQ ID NO: 61). The light chain CDRs of the 1B12 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1C7 antibody described herein.

>1C7 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCCGGGCACAACTACCCTTACTGGGGCCAAGGGACTCTGGTCACTGTCTC
TTCC (SEQ ID NO: 94)

>1C7 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGHNYPYWGQGTLVTVSS (SEQ ID NO: 95)

>1C7 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 96)

>1C7 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 97)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1C7 antibody have the following sequences: GYSITGGYS (SEQ ID NO: 49); IHYSGYT (SEQ ID NO: 56); and ARKDSGHNYPY (SEQ ID NO: 62). The light chain CDRs of the 1C7 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1C10 antibody described herein.

>1C10 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCAAGAACTTCCCTTACTGGGGCCAAGGGACTCTGGTCACTGTCTC
TTCC (SEQ ID NO: 98)

>1C10 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGKNFPYWGQGTLVTVSS (SEQ ID NO: 99)

>1C10 VL nucleic acid sequence GAAATTGTGTTGACGCAGTCTCCAGACTTTCAGTCTGTGACTCCAAAGGA AAAAGTCACCATCACCTGCAGGGCCAGTCAGAGTATCAGCGACCACTTAC ACTGGTACCAACAGAAACCTGATCAGTCTCCCAAGCTCCTCATCAAATAT GCTTCCCATGCCATTTCTGGGGTCCCATCGAGGTTCAGTGGCAGTGGGTC TGGGACAGACTTCACTCTCACCATCAATAGCCTAGAGGCTGAAGATGCTG CAACGTATTACTGTCAGCAGGGTCACAGTTTTCCGCTCACTTTCGGCGGA GGGACCAAGGTGGAGATCAAA (SEQ ID NO: 100)

>1C10 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 101)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1C10 antibody have the following sequences: GYSITGGYS (SEQ ID NO: 49); IHYSGYT (SEQ ID NO: 56); and ARKDSGKNFPY (SEQ ID NO: 63). The light chain CDRs of the 1C10 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1C12 antibody described herein.

>1C12 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCCAGTTGTTCCCTTACTGGGGCCAAGGGACTCTGGTCACTGTCTC
TTCC (SEQ ID NO: 102)

>1C12 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGQLFPYWGQGTLVTVSS (SEQ ID NO: 103)

>1C12 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 104)

>1C12 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 105)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1C12 antibody have the following sequences: GYSITGGYS (SEQ ID NO: 49); IHYSGYT (SEQ ID NO: 56); and ARKDSGQLFPY (SEQ ID NO: 64). The light chain CDRs of the 1C12 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1D10 antibody described herein.

>1D10 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCCACAACTTGCCTTACTGGGGCCAAGGGACTCTGGTCACTGTCTC
TTCC (SEQ ID NO: 106)

>1D10 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGHNLPYWGQGTLVTVSS (SEQ ID NO: 107)

>1D10 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 108)

>1D10 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 109)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1D10 antibody have the following sequences: GYSITGGYS (SEQ ID NO: 49); IHYSGYT (SEQ ID NO: 56); and ARKDSGHNLPY (SEQ ID NO: 65). The light chain CDRs of the 1D10 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11 N103D antibody described herein.

>1E11 N103D VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCGACTACTTCCCTTACTGGGGCCAAGGGACTCTGGTCACTGTCTC
TTCC (SEQ ID NO: 110)

>1E11 N103D VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGDYFPYWGQGTLVTVSS (SEQ ID NO: 111)

>1E11 N103D VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 112)

>1E11 N103D VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 113)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11 N103D antibody have the following sequences: GYSITGGYS (SEQ ID NO: 49); IHYSGYT (SEQ ID NO: 56); and ARKDSGDYFPY (SEQ ID NO: 66). The light chain CDRs of the 1E11 N103D antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1G12 antibody described herein.

>1G12 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCCGGGCGGTACTGGCCTTACTGGGGCCAAGGGACTCTGGTCACTGTCTC
TTCC (SEQ ID NO: 114)

>1G12 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGRYWPYWGQGTLVTVSS (SEQ ID NO: 115)

>1G12 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 116)

>1G12 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 117)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1G12 antibody have the following sequences: GYSITGGYS (SEQ ID NO: 49); IHYSGYT (SEQ ID NO: 56); and ARKDSGRYWPY (SEQ ID NO: 67). The light chain CDRs of the 1E11 N103D antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11.C1 antibody described herein.

>1E11.C1 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTTCCCGATCCGCTACGGGTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 118)

>1E11.C1 VH amino acid sequence
QVQLQESGPGLVKPSDTLSLTCAVSGFPIRYGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 119)

>1E11.C1 VL amino acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 120)

>1E11.C1 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 121)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C1 antibody have the following sequences: GFPIRYGYS (SEQ ID NO: 50); IHYSGYT (SEQ ID NO: 56); and ARKDSGNYFPY (SEQ ID NO: 58). The light chain CDRs of the 1E11.C1 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11.C2 antibody described herein.

>1E11.C2 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 122)

>1E11.C2 VH amino acid sequence
QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 123)

>1E11.C2 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 124)

>1E11.C2 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 125)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2 antibody have the following sequences: GYPIRFGYS (SEQ ID NO: 51); IHYSGYT (SEQ ID NO: 56); and ARKDSGNYFPY (SEQ ID NO: 58). The light chain CDRs of the 1E11.C1 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11.C3 antibody described herein.

>1E11.C3 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCCATCCGGCACGGGTACA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 126)

>1E11.C3 VH amino acid sequence
QVQLQESGPGLVKPSDTLSLTCAVSGYPIRHGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 127)

>1E11.C3 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 128)

>1E11.C3 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 129)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C3 antibody have the following sequences: GYPIRHGYS (SEQ ID NO: 52); IHYSGYT (SEQ ID NO: 56); and ARKDSGNYFPY (SEQ ID NO: 58). The light chain CDRs of the 1E11.C1 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11.C4 antibody described herein.

>1E11.C4 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTTCCCGATCGGCCAGGGGTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 130)

>1E11.C4 VH amino acid sequence
QVQLQESGPGLVKPSDTLSLTCAVSGFPIGQGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 131)

>1E11.C4 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 132)

>1E11.C4 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 133)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C4 antibody have the following sequences: GFPIGQGYS (SEQ ID NO: 53); IHYSGYT (SEQ ID NO: 56); and ARKDSGNYFPY (SEQ ID NO: 58). The light chain CDRs of the 1E11.C1 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11.C5 antibody described herein.

>1E11.C5 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCGATCTGGGGGGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCCGCCTCCACC (SEQ ID NO: 134)

>1E11.C5 VH amino acid sequence
QVQLQESGPGLVKPSDTLSLTCAVSGYPIWGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 135)

>1E11.C5 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 136)

>1E11.C5 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 137)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C5 antibody have the following sequences: GYPIWGGYS (SEQ ID NO: 54); IHYSGYT (SEQ ID NO: 56); and ARKDSGNYFPY (SEQ ID NO: 58). The light chain CDRs of the 1E11.C1 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11.C6 antibody described herein.

>1E11.C6 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCCATCGGCGGCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 138)

>1E11.C6 VH amino acid sequence
QVQLQESGPGLVKPSDTLSLTCAVSGYPIGGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 139)

>1E11.C6 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA (SEQ ID NO: 140)

>1E11.C6 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK (SEQ ID NO: 141)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C6 antibody have the following sequences: GYPIGGGYS (SEQ ID NO: 55); IHYSGYT (SEQ ID NO: 56); and ARKDSGNYFPY (SEQ ID NO: 58). The light chain CDRs of the 1E11.C1 antibody have the following sequences: QSISDH (SEQ ID NO: 68); YAS (SEQ ID NO: 69); and QQGHSFPLT (SEQ ID NO: 6).

An exemplary TLR4 monoclonal antibody is the 1E11.E1 antibody described herein.

>1E11.E1 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 142)

>1E11.E1 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 143)

>1E11.E1 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGGAACGACTTCCCGGTGACTTTCGGCGGA
GGGACCAAGGTGGAGATCAAA (SEQ ID NO: 144)

>1E11.E1 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGNDFPVTFGG
GTKVEIK (SEQ ID NO: 145)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E1 antibody have the following sequences: GYSITGGYS (SEQ ID NO: 49); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGNDFPVT (SEQ ID NO: 71).

An exemplary TLR4 monoclonal antibody is the 1E11.E2 antibody described herein.

>1E11.E2 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 146)

>1E11.E2 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 147)

>1E11.E2 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGGTACGACGAGCCGTTCACTTTCGGCGGA
GGGACCAAGGTGGAGATCAAA (SEQ ID NO: 148)

>1E11.E2 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGYDEPFTFGG
GTKVEIK (SEQ ID NO: 149)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E2 antibody have the following sequences: GYSITGGYS (SEQ ID NO: 49); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGYDEPFT (SEQ ID NO: 72).

An exemplary TLR4 monoclonal antibody is the 1E11.E3 antibody described herein.

>1E11.E3 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 150)
>1E11.E3 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 151)

>1E11.E3 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTTCCCGTTGACTTTCGGCGGA
GGGACCAAGGTGGAGATCAAA (SEQ ID NO: 152)

>1E11.E3 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGYDFPLTFGG
GTKVEIK (SEQ ID NO: 153)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E3 antibody have the following sequences: GYSITGGYS (SEQ ID NO: 49); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGYDFPLT (SEQ ID NO: 73).

An exemplary TLR4 monoclonal antibody is the 1E11.E4 antibody described herein.

>1E11.E4 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 154)

>1E11.E4 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 155)

>1E11.E4 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTACCCGCTCACTTTCGGCGGA
GGGACCAAGGTGGAGATCAAA (SEQ ID NO: 156)

>1E11.E4 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGYDYPLTFGG
GTKVEIK (SEQ ID NO: 157)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E4 antibody have the following sequences: GYSITGGYS (SEQ ID NO: 49); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGYDYPLT (SEQ ID NO: 74).

An exemplary TLR4 monoclonal antibody is the 1E11.E5 antibody described herein.

>1E11.E5 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 158)

>1E11.E5 VH amino acid sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 159)

>1E11.E5 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGAGTTCCCGTTGACTTTCGGCGGA
GGGACCAAGGTGGAGATCAAA (SEQ ID NO: 160)

>1E11.E5 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGYEFPLTFGG
GTKVEIK (SEQ ID NO: 161)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E5 antibody have the following sequences: GYSITGGYS (SEQ ID NO: 49); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGYEFPLT (SEQ ID NO: 75).

An exemplary TLR4 monoclonal antibody is the 1E11.C2E1 antibody described herein.

>1E11.C2E1 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 162)

>1E11.C2E1 VH amino acid sequence
QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 163)

>1E11.C2E1 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGGAACGACTTCCCGGTGACTTTCGGCGGA
GGGACCAAGGTGGAGATCAAA (SEQ ID NO: 164)

>1E11.C2E1 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGNDFPVTFGG
GTKVEIK (SEQ ID NO: 165)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2E1 antibody have the following sequences: GYPIRFGYS (SEQ ID NO: 51); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGNDFPVT (SEQ ID NO: 71).

An exemplary TLR4 monoclonal antibody is the 1E11.C2E3 antibody described herein.

>1E11.C2E3 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 166)

>1E11.C2E3 VH amino acid sequence
QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 167)

>1E11.C2E3 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTTCCCGTTGACTTTCGGCGGA
GGGACCAAGGTGGAGATCAAA (SEQ ID NO: 168)

>1E11.C2E3 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGYDFPLTFGG
GTKVEIK (SEQ ID NO: 169)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2E3 antibody have the following sequences: GYPIRFGYS (SEQ ID NO: 51); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGYDFPLT (SEQ ID NO: 73).

An exemplary TLR4 monoclonal antibody is the 1E11.C2E4 antibody described herein.

>1E11.C2E4 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 170)

>1E11.C2E4 VH amino acid sequence
QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 171)

>1E11.C2E4 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTACCCGCTCACTTTCGGCGGA
GGGACCAAGGTGGAGATCAAA (SEQ ID NO: 172)

>1E11.C2E4 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGYDYPLTFGG
GTKVEIK (SEQ ID NO: 173)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2E4 antibody have the following sequences: GYPIRFGYS (SEQ ID NO: 51); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGYDYPLT (SEQ ID NO: 74).

An exemplary TLR4 monoclonal antibody is the 1E11.C2E5 antibody described herein.

>1E11.C2E5 VH nucleic acid sequence
CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC (SEQ ID NO: 174)

>1E11.C2E5 VH amino acid sequence
QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS (SEQ ID NO: 175)

>1E11.C2E5 VL nucleic acid sequence
GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGAGTTCCCGTTGACTTTCGGCGGA
GGGACCAAGGTGGAGATCAAA (SEQ ID NO: 176)

>1E11.C2E5 VL amino acid sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGYEFPLTFGG
GTKVEIK (SEQ ID NO: 177)

The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2E5 antibody have the following sequences: GYPIRFGYS (SEQ ID NO: 51); IHYSGYT (SEQ ID NO: 56); and ARKDSGNYFPY (SEQ ID NO: 58). The light chain CDRs of the 1E11 antibody have the following sequences: QSISDH (SEQ ID NO: 68); YAS (SEQ ID NO: 69); and QQGYEFPLT (SEQ ID NO: 75).

In some embodiments, the TLR4 antibodies are formatted in an IgG isotype. In some embodiments, the TLR4 antibodies are formatted in an IgG1 isotype.

An exemplary IgG1-formatted antibody is the IgG1-formatted 1E11 antibody comprising the heavy chain sequence of SEQ ID NO: 178 and the light chain sequence of SEQ ID NO: 179, as shown below:

>1E11 Heavy Chain Amino Acid Sequence
QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG (S
EQ ID NO: 178)

>1E11 Light Chain Amino Acid Sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 179)

>1E11 Light Chain Nucleic Acid Sequence
ATGAGTGTGCCCACTCAGGTCCTGGGGTTGCTGCTGCTGTGGCTTACAGA
TGCCAGATGTGAAATTGTGTTGACGCAGTCTCCAGACTTTCAGTCTGTGA
CTCCAAAGGAAAAAGTCACCATCACCTGCAGGGCCAGTCAGAGTATCAGC
GACCACTTACACTGGTACCAACAGAAACCTGATCAGTCTCCCAAGCTCCT
CATCAAATATGCTTCCCATGCCATTTCTGGGGTCCCATCGAGGTTCAGTG
GCAGTGGGTCTGGGACAGACTTCACTCTCACCATCAATAGCCTAGAGGCT
GAAGATGCTGCAACGTATTACTGTCAGCAGGGTCACAGTTTTCCGCTCAC
TTTCGGCGGAGGGACCAAGGTGGAGATCAAACGTACGGTGGCTGCACCAT
CTGTCTTCATCTTCCCGCCATCTGATGAGCAGTTGAAATCTGGAACTGCC
TCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCA
CAGAGCAGGACAGCAAGGACAGCACCTACAGCCTCAGCAGCACCCTGACG
CTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGT
GTTAA (SEQ ID NO: 180)

>1E11 Heavy Chain Nucleic Acid Sequence
ATGGAATGGAGCTGGGTCTTTCTCTTCTTCCTGTCAGTAACTACAGGTGT
CCACCAGGTGCAGCTTCAGGAGTCCGGCCCAGGACTGGTGAAGCCTTCGG
ACACCCTGTCCCTCACCTGCGCTGTCTCTGGTTACTCCATCACCGGTGGT
TATAGCTGGCACTGGATACGGCAGCCCCCAGGGAAGGGACTGGAGTGGAT
GGGGTATATCCACTACAGTGGTTACACTGACTTCAACCCCTCCCTCAAGA
CTCGAATCACCATATCACGTGACACGTCCAAGAACCAGTTCTCCCTGAAG
CTGAGCTCTGTGACCGCTGTGGACACTGCAGTGTATTACTGTGCGAGAAA
AGATCCGTCCGACGCCTTTCCTTACTGGGGCCAAGGGACTCTGGTCACTG
TCTCTTCCGCCTCCACCAAGGGCCCATCGGTCTTCCCCCTGGCACCCTCC
TCCAAGAGCACCTCTGGGGGCACAGCGGCCCTGGGCTGCCTGGTCAAGGA
CTACTTCCCCGAACCGGTGACAGTCTCGTGGAACTCAGGAGCCCTGACCA
GCGGCGTGCACACCTTCCCGGCTGTCCTACAGTCCTCAGGACTCTACTCC
CTCAGCAGCGTGGTGACTGTGCCCTCCAGCAGCTTGGGCACCCAGACCTA
CATCTGCAACGTGAATCACAAGCCCAGCAACACCAAGGTGGACAAGAGAG
TTGAGCCCAAATCTTGTGACAAAACTCACACATGCCCACCGTGCCCAGCA
CCTGAACTCCTGGGGGGACCGTCAGTCTTCCTCTTCCCCCCAAAACCCAA
GGACACCCTCATGATCTCCCGGACCCCTGAGGTCACATGCGTGGTGGTGG
ACGTGAGCCACGAAGACCCTGAGGTCAAGTTCAACTGGTACGTGGACGGC
GTGGAGGTGCATAATGCCAAGACAAAGCCGCGGGAGGAGCAGTACAACAG
CACGTACCGTGTGGTCAGCGTCCTCACCGTCCTGCACCAGGACTGGCTGA
ATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGT
GTATACCCTGCCCCCATCTCGGGAGGAGATGACCAAGAACCAGGTCAGCC
TGACTTGCCTGGTCAAAGGCTTCTATCCCAGCGACATCGCCGTGGAGTGG
GAGAGCAACGGGCAGCCGGAGAACAACTACAAGACCACGCCTCCCGTGCT
GGACTCCGACGGCTCCTTCTTCCTCTATAGCAAGCTCACCGTGGACAAGT
CCAGGTGGCAGCAGGGGAACGTCTTCTCATGCTCCGTGATGCATGAGGCT
CTGCACAACCACTACACGCAGAAGAGCCTCTCCCTGTCTCCGGGTTAA (
SEQ ID NO: 181)

An exemplary IgG1-formatted antibody is the IgG1-formatted 1E11.C11 antibody comprising the heavy chain sequence of SEQ ID NO: 182 and the light chain sequence of SEQ ID NO: 183, as shown below:

>1E11.C1 Light Chain Amino Acid Sequence
EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 182)

>1E11.C1 Heavy Chain Amino Acid Sequence
QVQLQESGPGLVKPSDTLSLTCAVSGFPIRYGYSWHWIRQPPGKGLEWMG
YIHYSGYTDFNPLKTRITISRDTSKNQFSLKLSSVTAVDTAVYYCARKDS
GNYFPYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG (SE
Q ID NO: 183)

>1E11.C1 Light Chain Nucleic Acid Sequence
ATGAGTGTGCCCACTCAGGTCCTGGGGTTGCTGCTGCTGTGGCTTACAGA
TGCCAGATGTGAAATTGTGTTGACGCAGTCTCCAGACTTTCAGTCTGTGA
CTCCAAAGGAAAAAGTCACCATCACCTGCAGGGCCAGTCAGAGTATCAGC
GACCACTTACACTGGTACCAACAGAAACCTGATCAGTCTCCCAAGCTCCT
CATCAAATATGCTTCCCATGCCATTTCTGGGGTCCCATCGAGGTTCAGTG
GCAGTGGGTCTGGGACAGACTTCACTCTCACCATCAATAGCCTAGAGGCT
GAAGATGCTGCAACGTATTACTGTCAGCAGGGTCACAGTTTTCCGCTCAC
TTTCGGCGGAGGGACCAAGGTGGAGATCAAACGTACGGTGGCTGCACCAT
CTGTCTTCATCTTCCCGCCATCTGATGAGCAGTTGAAATCTGGAACTGCC
TCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCA
CAGAGCAGGACAGCAAGGACAGCACCTACAGCCTCAGCAGCACCCTGACG
CTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGT
GTTAA (SEQ ID NO: 184)

>1E11.C1 Heavy Chain Nucleic Acid Sequence
ATGGAATGGAGCTGGGTCTTTCTCTTCTTCCTGTCAGTAACTACAGGTGT
CCACCAGGTGCAGCTTCAGGAGTCCGGCCCAGGACTGGTGAAGCCTTCGG
ACACCCTGTCCCTCACCTGCGCTGTCTCTGGTTTCCCGATCCGCTACGGG
TATAGCTGGCACTGGATACGGCAGCCCCCAGGGAAGGGACTGGAGTGGAT
GGGGTATATCCACTACAGTGGTTACACTGACTTCAACCCCTCCCTCAAGA
CTCGAATCACCATATCACGTGACACGTCCAAGAACCAGTTCTCCCTGAAG
CTGAGCTCTGTGACCGCTGTGGACACTGCAGTGTATTACTGTGCGAGAAA
AGATTCGGGCAACTACTTCCCTTACTGGGGCCAAGGGACTCTGGTCACTG
TCTCTTCCGCCTCCACCAAGGGCCCATCGGTCTTCCCCCTGGCACCCTCC
TCCAAGAGCACCTCTGGGGGCACAGCGGCCCTGGGCTGCCTGGTCAAGGA
CTACTTCCCCGAACCGGTGACAGTCTCGTGGAACTCAGGAGCCCTGACCA
GCGGCGTGCACACCTTCCCGGCTGTCCTACAGTCCTCAGGACTCTACTCC
CTCAGCAGCGTGGTGACTGTGCCCTCCAGCAGCTTGGGCACCCAGACCTA
CATCTGCAACGTGAATCACAAGCCCAGCAACACCAAGGTGGACAAGAGAG
TTGAGCCCAAATCTTGTGACAAAACTCACACATGCCCACCGTGCCCAGCA
CCTGAACTCCTGGGGGGACCGTCAGTCTTCCTCTTCCCCCCAAAACCCAA
GGACACCCTCATGATCTCCCGGACCCCTGAGGTCACATGCGTGGTGGTGG
ACGTGAGCCACGAAGACCCTGAGGTCAAGTTCAACTGGTACGTGGACGGC
GTGGAGGTGCATAATGCCAAGACAAAGCCGCGGGAGGAGCAGTACAACAG
CACGTACCGTGTGGTCAGCGTCCTCACCGTCCTGCACCAGGACTGGCTGA
ATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGT
GTATACCCTGCCCCCATCTCGGGAGGAGATGACCAAGAACCAGGTCAGCC
TGACTTGCCTGGTCAAAGGCTTCTATCCCAGCGACATCGCCGTGGAGTGG
GAGAGCAACGGGCAGCCGGAGAACAACTACAAGACCACGCCTCCCGTGCT
GGACTCCGACGGCTCCTTCTTCCTCTATAGCAAGCTCACCGTGGACAAGT
CCAGGTGGCAGCAGGGGAACGTCTTCTCATGCTCCGTGATGCATGAGGCT
CTGCACAACCACTACACGCAGAAGAGCCTCTCCCTGTCTCCGGGTTAA (
SEQ ID NO: 185)

In some embodiments, TLR4 antibodies of the invention specifically bind human and/or cynomolgus TLR4/MD-2 complex, wherein the antibody binds to an epitope that includes one or more amino acid residues on human and/or cynomolgus TLR4 between residues 325 and 374 of SEQ ID NO: 11 (human) and SEQ ID NO: 77 (cynomolgus). Alternatively, the monoclonal antibody is an antibody that binds to the same epitope as 1A1, 1A6, 1B12, 1C7, 1C10, 1C12, 1D10, 1E11, 1E11 N103D, 1G12, 1E11.C1, 1E11.C2, 1E11.C3, 1E11.C4, 1E11.C5, 1E11.C6, 1E11.E1, 1E11.E2, 1E11.E3, 1E11.E4, 1E11.E5, 1E11.C2E1, 1E11.C2E3, 1E11.C2E4 and 1E11.C2E5.

The anti-TLR4 antibodies of the invention include an altered antibody in which at least the amino acid residue at EU position 325 and at least the amino acid residue at EU position 328 in the CH2 domain of the Fc portion of the antibody has been modified. For example, at least the amino acid residue at EU position 325 has been substituted with serine, and at least the amino acid residue at EU position 328 has been substituted with phenylalanine.

These anti-TLR4 antibodies with a modified Fc portion elicit modified effector functions e.g., a modified Fc receptor activity, as compared to an unaltered antibody. For example, the human Fc receptor is CD32A. In some embodiments, these anti-TLR4 antibodies elicit a prevention of proinflammatory mediators release following ligation to CD32A as compared to an unaltered antibody. Thus, these anti-TLR4 antibodies elicit a modified Fc receptor activity, such as the prevention of proinflammatory mediators release while retaining the ability to bind a target antigen. In some embodiments, these anti-TLR4 antibodies are neutralizing antibodies, wherein the anti-TLR4 antibody elicits a modified Fc receptor activity, while retaining the ability to neutralize one or more biological activities of a target antigen.

For example, anti-TLR4 antibodies of the invention include monoclonal antibodies that bind the human TLR4/MD-2 receptor complex. This receptor complex is activated by lipopolysaccharide (LPS), the major component of the outer membrane of gram-negative bacteria. The anti-TLR4 antibodies of the invention inhibit receptor activation and subsequent intracellular signaling via LPS. Thus, the anti-TLR4 antibodies neutralize the activation of the TLR4/MD-2 receptor complex. In particular, the invention provides anti-TLR4 antibodies that recognize the TLR4/MD-2 receptor complex expressed on the cell surface. These anti-TLR4 antibodies block LPS-induced and other TLR4 ligand-induced pro-inflammatory cytokine (e.g., IL-6, IL-8, TNFα) production. In addition, some anti-TLR4 antibodies of the invention also recognize TLR4 when not complexed with MD-2. The altered antibody is, e.g., a humanized antibody.

Monoclonal antibodies of the invention (e.g., murine monoclonal, humanized antibodies or fully human monoclonal antibodies) specifically bind TLR4. Also included in the invention are antibodies that bind to the same epitope as the antibodies described herein. For example, antibodies of the invention that specifically bind TLR4 and/or the TLR4/MD-2 complex bind to an epitope that includes one or more amino acid residues on human TLR4 (SEQ ID NO: 11).

In some embodiments, the TLR4 antibody is a surrogate TLR4 antibody formatted in a mouse IgG2a κ Fc isotype. This chimeric surrogate mAb is referred to as 5E3.

An exemplary TLR4 monoclonal antibody is the “5E3” or “5E3 Rat_VH_mIgG2a FC IGHV9-4” or “5E3 Chimeric Rat_mIgG2a” antibody described herein is shown below. The amino acids encompassing the complementarity determining regions (CDR) are as defined by M.P. Lefranc (See Lefranc, M.-P., Current Protocols in Immunology, J. Wiley and Sons, New York supplement 40, A1.P.1-A.1P.37 (2000) LIGM:230). CDR sequences are shown in bolded and underlined font below.

>5E3 heavy chain amino acid sequence
QIQLVQSGPELKKPGESVKISCKASGYTFTDYV MYWVKQAPGKGLKWMGW
INTNTGKP TYADDFKGRFVFSLEASASTANLQISNLKNEDTATYFCTRGN YPGLRVMDA WGQGASVTVSSAKTTAPSVYPLAPVCGDTTGSSVTLGCLVK
GYFPEPVTLTWNSGSLSSGVHTFPAVLQSDLYTLSSSVTVTSSTWPSQSI
TCNVAHPASSTKVDKKIEPRGPTIKPCPPCKCPAPNLLGGPSVFIFPPKI
KDVLMISLSPIVTCVVVDVSEDDPDVQISWFVNNVEVHTAQTQTHREDYN
STLRVVSALPIQHQDWMSGKEFKCKVNNKDLPAPIERTISKPKGSVRAPQ
VYVLPPPEEEMTKKQVTLTCMVTDFMPEDIYVEWTNNGKTELNYKNTEPV
LDSDGSYFMYSKLRVEKKNWVERNSYSCSWHEGLHNHHTTKSFSRTPGK
(SEQ ID NO: 340)

>5E3 VH amino acid sequence
QIQLVQSGPELKKPGESVKISCKASGYTFTDYV MYWVKQAPGKGLKWMGW
INTNTGKP TYADDFKGRFVFSLEASASTANLQISNLKNEDTATYFCTRGN YPGLRVMDA WGQGASVTVSS(SEQ ID NO: 341)

>5E3 light chain amino acid sequence
DIQMTQSPSSMSVSLGDTVTITCRASQDVGIY VNWFQQKPGKSPRRLIFR AT NLADGVPSRFSGSRSGSDYSLTISSLGSEDVAGYHCLQYDEYPLT FGS
GTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC (SEQ ID NO: 342)

>5E3 VL amino acid sequence
DIQMTQSPSSMSVSLGDTVTITCRASQDVGIY VNWFQQKPGKSPRRLIFR AT NLADGVPSRFSGSRSGSDYSLTISSLGSEDVAGYHCLQYDEYPLT FGS
GTKLEIK (SEQ ID NO: 343)

The heavy chain CDRs of the 5E3 antibody have the following sequences: GYTFTDYV (SEQ ID NO: 344); INTNTGKP (SEQ ID NO: 345); and TRGNYPGLRVMDA (SEQ ID NO: 346). The light chain CDRs of the 5E3 antibody have the following sequences: QDVGIY (SEQ ID NO: 347); RAT (SEQ ID NO: 348); and LQYDEYPLT (SEQ ID NO: 349).

Those skilled in the art will recognize that it is possible to determine, without undue experimentation, if a monoclonal antibody (e.g., a murine monoclonal or humanized antibody) has the same specificity as a monoclonal antibody described herein by ascertaining whether the former prevents the latter from binding to the TLR4/MD-2 complex or to TLR4 when not complexed to MD-2. If the monoclonal antibody being tested competes with the monoclonal antibody of the invention, as shown by a decrease in binding by the monoclonal antibody of the invention, then the two monoclonal antibodies bind to the same, or a closely related, epitope. An alternative method for determining whether a monoclonal antibody has the specificity of monoclonal antibody of the invention is to pre-incubate the monoclonal antibody of the invention with the TLR4/MD-2 complex or a soluble TLR4 protein (with which it is normally reactive), and then add the monoclonal antibody being tested to determine if the monoclonal antibody being tested is inhibited in its ability to bind the TLR4/MD-2 complex or to bind TLR4 and TLR4 complexed with MD-2. If the monoclonal antibody being tested is inhibited then, in all likelihood, it has the same, or functionally equivalent, epitopic specificity as the monoclonal antibody of the invention.

### HUIP-10 Antibodies

The huIP-10 antibodies are, for example, IP-10 antagonists or inhibitors that modulate at least one biological activity of IP-10. Biological activities of IP-10 include, for example, binding the IP-10 receptor (CXCR3), IP-10 induced calcium flux, IP-10 induced cell chemotaxis, IP-10 binding to glycosaminoglycan, and IP-10 oligomerization. For example, the huIP-10 antibodies completely or partially inhibit IP-10 activity by partially or completely blocking the binding of IP-10 to the IP-10 receptor (CXCR3). The IP-10 antibodies are considered to completely inhibit IP-10 activity when the level of IP-10 activity in the presence of the huIP-10 antibody is decreased by at least 95%, e.g., by 96%, 97%, 98%, 99% or 100% as compared to the level of IP-10 activity in the absence of binding with a huIP-10 antibody described herein. The IP-10 antibodies are considered to partially inhibit IP-10 activity when the level of IP-10 activity in the presence of the huIP-10 antibody is decreased by less than 95%, e.g., 10%, 20%, 25%, 30%, 40%, 50%, 60%, 75%, 80%, 85% or 90% as compared to the level of IP-10 activity in the absence of binding with a huIP-10 antibody described herein.

The huIP-10 antibodies of the invention are produced by immunizing an animal with IP-10, such as, for example, murine or human IP-10 or an immunogenic fragment, derivative or variant thereof. Alternatively, the animal is immunized with cells transfected with a vector containing a nucleic acid molecule encoding IP-10, such that IP-10 is expressed and associated with the surface of the transfected cells. Alternatively, the antibodies are obtained by screening a library that contains antibody or antigen binding domain sequences for binding to IP-10. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”).

huIP-10 antibodies of the invention include, for example, the heavy chain complementarity determining regions (CDRs) shown below in Table 3, the light chain CDRs shown in Table 4, and combinations thereof.

TABLE 3

VH CDR sequences from antibody clones that bind and neutralize IP-10

| | | | |
|---|---|---|---|
| Clone ID | Heavy CDR1 | Heavy CDR2 | Heavy CDR3 |
| NI-0801 | NSGIH.... | VISY.....DGSNKYYADSVKG | LRDNAEYT...............DY |
| CF1N1R3P4_C7 | NSGIH.... | VISY.....DGSNKFYADSVKG | LRDNAEYT...............DY |
| CF1H1R3P3_G11 | NSGIH.... | VISY.....DGSNKFYADSVKG | LRDNGEYL...............DY |
| CF1A11R3P3_F3 | NSGIH.... | VISY.....DGSNKFYADSVKG | LRDNGEYL...............DY |
| CF1H1R3P4_B5 | NSGIH.... | VISY.....DGSNKFYADSVKG | LRDNGEYL...............DY |
| CC21R3P1_F1 | NSGIH.... | VISY.....DGSNKFYADSVKG | DGSESEYL...............DY |
| CC21R3P1_B9 | SYGMH.... | VISY.....DGSNKYYADSVKG | DGGWYDWYF..............DL |
| CB21R3P3_E5 | SYGMH.... | VISY.....DGSIKYYADSVKG | APDGHQL................DY |
| CC21R3P1_H6 | NYGMH.... | VISY.....DGSNRYYADSVKG | DAGGPL.................DY |
| CC21R3P5_C5 | TYGMH.... | VISY.....DGGTKYYADSVKG | DLGDLPPGL..............DY |
| CB1R3P4_D3 | SYGMH.... | VISY.....DGSIKYYADSVKG | AGYSTDWHP..............DY |
| CB2R2P4_C3 | TSGMSVI.. | RID......SDDEKHYNTSLKT | LRAGSGPYVF.............DS |
| CC21R3P4_F4 | NYGMH.... | VISY.....DGSNRYYADSVKG | DAGGPL.................DY |
| CC21R3P1_C1a | SYGMH.... | VISY.....DGSNKYYADSVKG | DEFDAF.................DI |
| CC21R3P3_C1 | SYGMH.... | VISY.....DGSIKYYADSVKG | DWGFSGSLTF.............DY |
| CC21R3P1_E7 | TYGMH.... | VISY.....DGGTKYYADSVKG | DLGDLPPGL..............DY |
| CE7C1R3H8_J9 | TYGMH.... | VISY.....DGGTKYYADSVKG | DLGDLPPGL..............DY |
| CB21R3P1_F1 | SYGMH.... | VISY.....DGSIKYYADSVKG | VMGTDPHSYYYM...........DV |
| CC21R3P1_A2 | DTYMN.... | SIY......SDDSTYYADSVKG | DKEYVTSTGGAYYYFYYM.....DV |
| CB21R3P6_G7 | SFSIT.... | EITP.....MFGIANYAQKFQG | DGRFDVSDLLTDKPKVTINYNGMDV |

TABLE 4

VL CDR sequences from antibody clones that bind and neutralize IP-10

| | | | |
|---|---|---|---|
| Clone ID | Light CDR1 | Light CDR2 | Light CDR3 |
| NI-0801 | TGSGGS......IASNYVQ | ED.....NQRPS | QSYDPLPV........WV |
| CF1N1R3P4_C7 | TGSGGS......IDRNYVQ | ED.....NQRPS | QSYDPLPV........WV |
| CF1H1R3P3_G11 | TGSGGS......IDRNYVQ | ED.....NQRPS | QSYDPLPV........WV |
| CF1A11 R3P3_F3 | TGSGGS......IDRNYVQ | ED.....NQRPS | QSYDSINL........WV |
| CF1H1R3P4_B5 | TGSGGS......IDRNYVQ | ED.....NQRPS | QSYVETPE........WV |
| CC21R3P1_F1 | TGSGGS......IDRNYVQ | ED.....NQRPS | QSYDSINL........WV |
| CC21R3P1_B9 | QGDS........LTSYYAS | GN.....DNRPS | GSRDSSGYQ ....... VV |
| CB21R3P3_E5 | TGSSGS......IASNYVQ | ED.....DQRPS | QSYVSSK.........WV |
| CC21R3P1_H6 | GGDN........IGRKSVH | DD.....TDRPS | QVWDSSIDHS......WV |
| CC21R3P5_C5 | GGSS........IESKSVH | KD.....SNRPS | QVWDSSTG........VV |
| CB1R3P4_D3 | QGDS........LRSYYAS | GK.....NNRPS | NSRDSSGNH.......VV |
| CB2R2P4_C3 | SGSSSN......IGSNTVN | NN.....DQRPS | ASWDDSLNG.......RV |
| CC21 R3P4_F4 | GGNN........IGDKSVQ | DD.....SDRPS | QVWDSSSDHPE.....VV |
| CC21R3P1_C1a | GGNN........IGSRSVH | YD.....SDRPS | QVWDTSSGH.......YV |
| CC21R3P3_C1 | GGNN........IGSKSVH | YD.....SDRPS | QVWDSSSDH.......VV |
| CC21R3P1_E7 | SGSSSN......IGSNTVN | TN.....NQRPS | AAWDDSLNGN......VV |
| CE7C1R3H8_J9 | SGSSSN......IGSNTVN | TN.....NQRPS | AAWDDSSEPR......VV |
| CB21R3P1_F1 | QGDS........LRSYYAS | GK.....NNRPS | NSRDSSGNH.......VL |
| CC21R3P1_A2 | SGSSSN......IGSDTVN | NN.....NQRPS | AAWDDSLNG.......LV |
| CB21R3P6_G7 | SGSSSN......IGSNTVN | NN.....DQRPS | ASWDDSLNG.......RV |

An exemplary huIP-10 monoclonal antibody is the NI-0801 antibody described herein. As shown below, the NI-0801 antibody includes a heavy chain variable region (SEQ ID NO: 198) encoded by the nucleic acid sequence shown in SEQ ID NO: 197, and a light chain variable region (SEQ ID NO: 199) encoded by the nucleic acid sequence shown in SEQ ID NO: 200.

>NI-0801 VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAACTCTGGCA
TACACTGGGTCCGCCAGGCTCCAGGCAAGGGACTGGAGTGGGTGGCAGTT
ATATCATATGATGGAAGTAACAAATACTACGCAGACTCCGTGAAGGGCCG
ATTCACCATCTCCAGAGACAACTCCAAGAACACTCTGTATCTGCAAATGA
ACAGCCTGAGAGCTGAGGACACGGCTGTCTATTATTGTGCAAGATTGAGG
GATAATGCGGAGTATACTGATTACTGGGGCCAGGGAACCCTGGTCACCGT
CTCGAGT (SEQ ID NO: 197)

>NI-0801 VH amino acid sequence
QVQLVESGGGVVQPGRSLRLSCAASGFTFSNSGIHWVRQAPGKGLEWVAV
ISYDGSNKYYADSVKGRFTISRDNSKNTLYLQMNSLRAEDTAVYYCARLR
DNAEYTDYWGQGTLVTVSS (SEQ ID NO: 198)

>NI-0801 VL nucleic acid sequence
AATTTTATGCTGACTCAGCCCCACTCTGTGTCGGAGTCTCCGGGGAAGAC
GGTAACCATCTCCTGCACCGGCAGCGGTGGCAGCATTGCCAGCAACTATG
TGCAGTGGTACCAACAGCGCCCGGGCAGTTCCCCCACCACTGTCATCTAT
GAGGATAACCAGAGACCCTCTGGGGTCCCTGATCGGTTCTCTGGCTCCAT
CGACAGCTCCTCCAATTCTGCCTCCCTCACCATCTCTGGGCTGAAGACTG
AGGACGAGGCTGACTACTACTGTCAGTCTTATGATCCGCTTCCGGTGTGG
GTTTTCGGCGGAGGGACCAAGCTGACCGTCCTAG (SEQ ID NO: 199)

>NI-0801 VL amino acid sequence
NFMLTQPHSVSESPGKTVTISCTGSGGSIASNYVQWYQQRPGSSPTTVIY
EDNQRPSGVPDRFSGSIDSSSNSASLTISGLKTEDEADYYCQSYDPLPVW
VFGGGTKLTVL (SEQ ID NO: 200)

The nucleic acid and amino acid sequences of the IgG1 reformatted NI-0801 antibody is shown below:

>NI-0801 Light Chain Amino Acid Sequence
NFMLTQPHSVSESPGKTVTISCTGSGGSIASNYVQWYQQRPGSSPTTVIY
EDNQRPSGVPDRFSGSIDSSSNSASLTISGLKTEDEADYYCQSYDPLPVW
VFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS (SEQ ID NO :328)

>NI-0801 Heavy Chain Amino Acid Sequence
QVQLVESGGGVVQPGRSLRLSCAASGFTFSNSGIHWVRQAPGKGLEWVAV
ISYDGSNKYYADSVKGRFTISRDNSKNTLYLQMNSLRAEDTAVYYCARLR
DNAEYTDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
(SEQ ID NO :329)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the NI-0801 antibody have the following sequences: NSGIH (SEQ ID NO: 201); VISYDGSNKYYADSVKG (SEQ ID NO: 202); and LRDNAEYTDY (SEQ ID NO: 203). The light chain CDRs of the NI-0801 antibody have the following sequences: TGSGGSIASNYVQ (SEQ ID NO: 204); EDNQRPS (SEQ ID NO: 205); and QSYDPLPVWV (SEQ ID NO: 206).

An exemplary huIP-10 monoclonal antibody is the CF1N1R3P4_C7 (“C7”) antibody described herein. As shown below, the C7 antibody includes a heavy chain variable region (SEQ ID NO: 208) encoded by the nucleic acid sequence shown in SEQ ID NO: 207, and a light chain variable region (SEQ ID NO: 210) encoded by the nucleic acid sequence shown in SEQ ID NO: 209.

>CF1N1R3P4 C7 VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAACTCTGGCA
TACACTGGGTCCGCCAGGCTCCAGGCAAGGGACTGGAGTGGGTGGCAGTT
ATATCATATGATGGAAGTAACAAATTCTACGCAGACTCCGTGAAGGGCCG
ATTCACCATCTCCAGAGACAACTCCAAGAACACTCTGTATCTGCAAATGA
ACAGCCTGAGAGCTGAGGACACGGCTGTCTATTATTGTGCAAAATTGAGG
GATAATGCGGAGTATACTGATTACTGGGGCCAGGGAACCCTGGTCACCGT
CTCGAGTG (SEQ ID NO: 207)

>CF1N1R3P4 C7 VH amino acid sequence
QVQLVESGGGVVQPGRSLRLSCAASGFTFSNSGIHWVRQAPGKGLEWVAV
ISYDGSNKFYADSVKGRFTISRDNSKNTLYLQMNSLRAEDTAVYYCAKLR
DNAEYTDYWGQGTLVTVSS (SEQ ID NO: 208)

> CF1N1R3P4 C7 VL nucleic acid sequence
AATTTTATGCTGACTCAGCCCCACTCTGTGTCGGAGTCTCCGGGGAAGAC
GGTGACCATCTCCTGCACCGGCAGCGGTGGCAGCATTGACAGAAACTATG
TGCAGTGGTACCAGCAGCGCCCGGGCAGTTCCCCCACCACTGTGATCTAT
GAGGATAACCAAAGACCCTCTGGGGTCCCGGATCGATTCTCTGGCTCCAT
CGACAGCTCCTCCAACTCTGCCTCCCTCACCATCTCTGGACTAAAAACTG
AAGACGAGGCTGACTACTACTGTCAGTCTTATGATCCGCTTCCGGTGTGG
GTTTTCGGCGGAGGGACCAAGCTCACCGTCCTA (SEQ ID NO: 209)

> CF1N1R3P4 C7 VL amino acid sequence
NFMLTQPHSVSESPGKTVTISCTGSGGSIDRNYVQWYQQRPGSSPTTVIY
EDNQRPSGVPDRFSGSIDSSSNSASLTISGLKTEDEADYYCQSYDPLPVW
VFGGGTKLTVL (SEQ ID NO: 210)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the C7 antibody have the following sequences: NSGIH (SEQ ID NO: 201); VISYDGSNKFYADSVKG (SEQ ID NO: 211); and LRDNAEYTDY (SEQ ID NO: 203). The light chain CDRs of the C7 antibody have the following sequences: TGSGGSIDSNYVQ (SEQ ID NO: 212); EDNQRPS (SEQ ID NO: 205); and QSYDPLPVWV (SEQ ID NO: 206).

An exemplary huIP-10 monoclonal antibody is the CF1H1R3P3_G11 (“G11”) antibody described herein. As shown below, the G11 antibody includes a heavy chain variable region (SEQ ID NO: 214) encoded by the nucleic acid sequence shown in SEQ ID NO: 213, and a light chain variable region (SEQ ID NO: 216) encoded by the nucleic acid sequence shown in SEQ ID NO: 215.

> CF1H1R3P3 _G11 VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAACTCTGGCA
TACACTGGGTCCGCCAGGCTCCAGGCAAGGGACTGGAGTGGGTGGCAGTT
ATATCATATGATGGAAGTAACAAATTCTACGCAGACTCCGTGAAGGGCCG
ATTCACCATCTCCAGAGACAACTCCAAGAACACTCTGTATCTGCAAATGA
ACAGCCTGAGAGCTGAGGACACGGCTGTCTATTATTGTGCAAAATTGAGG
GATAATGGTGAGTACTTAGACTACTGGGGCCAGGGAACCCTGGTCACCGT
CTCGAGT (SEQ ID NO: 213)

> CF1H1R3P3 _G11 VH amino acid sequence
QVQLVESGGGVVQPGRSLRLSCAASGFTFSNSGIHWVRQAPGKGLEWVAV
ISYDGSNKFYADSVKGRFTISRDNSKNTLYLQMNSLRAEDTAVYYCAKLR
DNGEYLDYWGQGTLVTVSS (SEQ ID NO: 214)

> CF1H1R3P3 G11 VL nucleic acid sequence
AATTTTATGCTGACTCAGCCCCACTCTGTGTCGGAGTCTCCGGGGAAGAC
GGTGACCATCTCCTGCACCGGCAGCGGTGGCAGCATTGACAGAAACTATG
TGCAGTGGTACCAGCAGCGCCCGGGCAGTTCCCCCACCACTGTGATCTAT
GAGGATAACCAAAGACCCTCTGGGGTCCCGGATCGATTCTCTGGCTCCAT
CGACAGCTCCTCCAACTCTGCCTCCCTCACCATCTCTGGACTAAAAACTG
AAGACGAGGCTGACTACTACTGTCAGTCTTATGATCCGCTTCCGGTGTGG
GTTTTCGGCGGAGGGACCAAGCTCACCGTCCTA (SEQ ID NO: 215)

> CF1H1R3P3 G11 VL amino acid sequence
NFMLTQPHSVSESPGKTVTISCTGSGGSIDRNYVQWYQQRPGSSPTTVIY
EDNQRPSGVPDRFSGSIDSSSNSASLTISGLKTEDEADYYCQSYDPLPVW
VFGGGTKLTVL (SEQ ID NO: 210)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the G11 antibody have the following sequences: NSGIH (SEQ ID NO: 201); VISYDGSNKFYADSVKG (SEQ ID NO: 211); and LRDNGEYLDY (SEQ ID NO: 217). The light chain CDRs of the G11 antibody have the following sequences: TGSGGSIDSNYVQ (SEQ ID NO: 212); EDNQRPS (SEQ ID NO: 205); and QSYDPLPVWV (SEQ ID NO: 206).

An exemplary huIP-10 monoclonal antibody is the CF1H1R3P4_B5 (“B5”) antibody described herein. As shown below, the B5 antibody includes a heavy chain variable region (SEQ ID NO: 214) encoded by the nucleic acid sequence shown in SEQ ID NO: 213, and a light chain variable region (SEQ ID NO: 216) encoded by the nucleic acid sequence shown in SEQ ID NO: 215.

> CF1H1R3P4 B5 VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAACTCTGGCA
TACACTGGGTCCGCCAGGCTCCAGGCAAGGGACTGGAGTGGGTGGCAGTT
ATATCATATGATGGAAGTAACAAATTCTACGCAGACTCCGTGAAGGGCCG
ATTCACCATCTCCAGAGACAACTCCAAGAACACTCTGTATCTGCAAATGA
ACAGCCTGAGAGCTGAGGACACGGCTGTCTATTATTGTGCAAAATTGAGG
GATAATGGTGAGTACTTAGACTACTGGGGCCAGGGAACCCTGGTCACCGT
CTCGAGT (SEQ ID NO: 213)

> CF1H1R3P4 B5 VH amino acid sequence
QVQLVESGGGVVQPGRSLRLSCAASGFTFSNSGIHWVRQAPGKGLEWVAV
ISYDGSNKFYADSVKGRFTISRDNSKNTLYLQMNSLRAEDTAVYYCAKLR
DNGEYLDYWGQGTLVTVSS (SEQ ID NO: 214)

> CF1H1R3P4 B5 VL nucleic acid sequence
AATTTTATGCTGACTCAGCCCCACTCTGTGTCGGAGTCTCCGGGGAAGAC
GGTGACCATCTCCTGCACCGGCAGCGGTGGCAGCATTGACAGAAACTATG
TGCAGTGGTACCAGCAGCGCCCGGGCAGTTCCCCCACCACTGTGATCTAT
GAGGATAACCAAAGACCCTCTGGGGTCCCGGATCGATTCTCTGGCTCCAT
CGACAGCTCCTCCAACTCTGCCTCCCTCACCATCTCTGGACTAAAAACTG
AAGACGAGGCTGACTACTACTGTCAGTCTTATGTGGAGACGCCTGAGTGG
GTTTTCGGCGGAGGGACCAAGCTCACCGTCCTAG (SEQ ID NO: 218)

> CF1H1R3P4 B5 VL amino acid sequence
NFMLTQPHSVSESPGKTVTISCTGSGGSIDRNYVQWYQQRPGSSPTTVIY
EDNQRPSGVPDRFSGSIDSSSNSASLTISGLKTEDEADYYCQSYVETPEW
VFGGGTKLTVL (SEQ ID NO: 219)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the B5 antibody have the following sequences: NSGIH (SEQ ID NO: 201); VISYDGSNKFYADSVKG (SEQ ID NO: 211); and LRDNGEYLDY (SEQ ID NO: 217). The light chain CDRs of the B5 antibody have the following sequences: TGSGGSIDSNYVQ (SEQ ID NO: 212); EDNQRPS (SEQ ID NO: 205); and QSYVETPEWV (SEQ ID NO: 220).

An exemplary huIP-10 monoclonal antibody is the CF1A11R3P3_F3 (“F3”) antibody described herein. As shown below, the F3 antibody includes a heavy chain variable region (SEQ ID NO: 214) encoded by the nucleic acid sequence shown in SEQ ID NO: 213, and a light chain variable region (SEQ ID NO: 224) encoded by the nucleic acid sequence shown in SEQ ID NO: 223.

>CF1A11R3P3F3 VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAACTCTGGCA
TACACTGGGTCCGCCAGGCTCCAGGCAAGGGACTGGAGTGGGTGGCAGTT
ATATCATATGATGGAAGTAACAAATTCTACGCAGACTCCGTGAAGGGCCG
ATTCACCATCTCCAGAGACAACTCCAAGAACACTCTGTATCTGCAAATGA
ACAGCCTGAGAGCTGAGGACACGGCTGTCTATTATTGTGCAAAATTGAGG
GATAATGGTGAGTACTTAGACTACTGGGGCCAGGGAACCCTGGTCACCGT
CTCGAGT (SEQ ID NO : 213)

>CF1A11R3P3F3 VH amino acid sequence
QVQLVESGGGVVQPGRSLRLSCAASGFTFSNSGIHWVRQAPGKGLEWVAV
ISYDGSNKFYADSVKGRFTISRDNSKNTLYLQMNSLRAEDTAVYYCAKLR
DNGEYLDYWGQGTLVTVSS (SEQ ID NO : 214)

>CF1A11R3P3F3 VL nucleic acid sequence
AATTTTATGCTGACTCAGCCCCACTCTGTGTCGGAGTCTCCGGGGAAGAC
GGTGACCATCTCCTGCACCGGCAGCGGTGGCAGCATTGACAGAAACTATG
TGCAGTGGTACCAGCAGCGCCCGGGCAGTGCCCCCATCACTGTGATCTAT
GAGGATAACCAAAGACCCTCTGGGGTCCCGGATCGATTCTCTGGCTCCAT
CGACAGCTCCTCCAACTCTGCCTCCCTCACCATCTCTGGACTACGGACTG
ACGACGAGGCTGACTACTACTGTCAGTCTTATGATAGCATCAATCTTTGG
GTTTTCGGCGGAGGGACCAAGGTCACCGTCCTAGG (SEQ ID NO : 2
23)

>CF1A11R3P3F3 VL amino acid sequence
NFMLTQPHSVSESPGKTVTISCTGSGGSIDRNYVQWYQQRPGSAPITVIY
EDNQRPSGVPDRFSGSIDSSSNSASLTISGLRTDDEADYYCQSYDSINLW
VFGGGTKVTVL (SEQ ID NO : 224)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the F3 antibody have the following sequences: NSGIH (SEQ ID NO: 201); VISYDGSNKFYADSVKG (SEQ ID NO: 211); and LRDNGEYLDY (SEQ ID NO: 217). The light chain CDRs of the F3 antibody have the following sequences: TGSGGSIDSNYVQ (SEQ ID NO: 212); EDNQRPS (SEQ ID NO: 205); and QSYDSINLWV (SEQ ID NO: 225).

An exemplary huIP-10 monoclonal antibody is the CB1R3P4_D3 (“D3”) antibody described herein. As shown below, the D3 antibody includes a heavy chain variable region (SEQ ID NO: 227) encoded by the nucleic acid sequence shown in SEQ ID NO: 226, and a light chain variable region (SEQ ID NO: 229) encoded by the nucleic acid sequence shown in SEQ ID NO: 228.

>CB1R3P4D3 VH nucleic acid sequence
CAGGTGCAGCTGGTGCAGTTTGGGGGAGGCGTGGTCCAGCCTGGGAGGTC
CTTGAGACTCTCCTGTGCAGCCTCTGGATTCACCTTCAGTAGCTATGGCA
TGCACTGGGTCCGCCAGGCTCCAGGCAAGGGGCTGGAGTGGGTGGCAGTT
ATATCATATGATGGAAGTATTAAATACTATGCAGACTCCGTGAAGGGCCG
ATTCACCATCTCCAGAGAAAATGCCAAGAACTCCGTGTATCTGCAAATGG
ACAGCCTGAGAGTCGGGGACACGGCTGTGTATTACTGTACAAGAGCCGGG
TATAGTACTGACTGGCATCCCGACTACTGGGGCCAGGGGACAATGGTCAC
CGTCTCGAGT (SEQ ID NO :226)

>CB1R3P4D3 VH amino acid sequence
QVQLVQFGGGVVQPGRSLRLSCAASGFTFSSYGMHWVRQAPGKGLEWVAV
ISYDGSIKYYADSVKGRFTISRENAKNSVYLQMDSLRVGDTAVYYCTRAG
YSTDWHPDYWGQGTMVTVSS (SEQ ID NO :227)

>CB1R3P4D3 VL nucleic acid sequence
TCTTCTGAGCTGACTCAGGACCCTGCTGTGTCTGTGGCCTTGGGACAGAC
AGTCAGGATCACATGCCAAGGAGACAGCCTCAGAAGCTATTATGCAAGCT
GGTACCAGCAGAAGCCAGGACAGGCCCCTGTACTTGCCATCTATGGTAAA
AACAACCGGCCCTCAGGGATCCCAGACCGATTCTCTGGCTCCAGCTCAGG
AAACACAGCTTCCTTGACCATCACTGGGGCTCAGGCGGAAGATGAGGCTG
ACTATTACTGTAACTCCCGGGACAGCAGTGGTAACCATGTGGTATTCGGC
GGAGGGACCAAGCTGACCGTCCTA (SEQ ID NO :228)

>CB1R3P4D3 VL amino acid sequence
SSELTQDPAVSVALGQTVRITCQGDSLRSYYASWYQQKPGQAPVLAIYGK
NNRPSGIPDRFSGSSSGNTASLTITGAQAEDEADYYCNSRDSSGNHWFGG
GTKLTVL (SEQ ID NO :229)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the D3 antibody have the following sequences: SYGMH (SEQ ID NO: 230); VISYDGSIKYYADSVKG (SEQ ID NO: 231); and AGYSTDWHPDY (SEQ ID NO: 232). The light chain CDRs of the D3 antibody have the following sequences: QGDSLRSYYAS (SEQ ID NO: 233); GKNNRPS (SEQ ID NO: 234); and NSRDSSGNHVV (SEQ ID NO: 235).

An exemplary huIP-10 monoclonal antibody is the CB2R2P4_C3 (“C3”) antibody described herein. As shown below, the C3 antibody includes a heavy chain variable region (SEQ ID NO: 237) encoded by the nucleic acid sequence shown in SEQ ID NO: 236, and a light chain variable region (SEQ ID NO: 239) encoded by the nucleic acid sequence shown in SEQ ID NO: 238.

>CB2R2P4 C3 VH nucleic acid sequence
CAGGTCACCTTGAGGGAGTCTGGTCCTGCGCTGGTGAAACCCACACAGAC
CCTCACACTGACCTGCACCTTCTCTGGATTCTCACTCACCACTAGTGGAA
TGTCTGTGATTTGGATCCGTCAGCCCCCAGGGAAGGCCCTGGAGTGGCTT
GCACGCATTGATTCGGATGACGAGAAACACTACAACACATCTCTGAAGAC
CAGGCTCGCCATCTCCAAGGACACCTCCAAAAACCAGGTGGTCCTTACAA
TGACCAACATGGACCCTGTGGACACAGGCACCTATTACTGTGCACGGCTT
CGGGCTGGTTCAGGTCCATATGTTTTTGACTCCTGGGGGCAAGGGACCAC
GGTCACCGTCTCGAGT (SEQ ID NO :236)

>CB2R2P4 C3 VH amino acid sequence
QVTLRESGPALVKPTQTLTLTCTFSGFSLTTSGMSVIWIRQPPGKALEWL
ARIDSDDEKHYNTSLKTRLAISKDTSKNQWLTMTNMDPVDTGTYYCARLR
AGSGPYVFDSWGQGTTVTVSS (SEQ ID NO :237)

>CB2R2P4 C3 VLnucleic acid sequence
CAGTCTGTGCTGACTCAGCCACCCTCAGCGTCTGGGACCCCCGGGCAGAG
GGTCACCATCTCTTGTTCTGGAAGCAGCTCCAACATCGGGAGTAACACTG
TAAACTGGTACCAGCGACTCCCAGGAGCGGCCCCCCAACTCCTCATCTAC
AATAATGACCAGCGGCCCTCAGGGATCCCTGACCGATTCTCTGGCTCCAA
GTCTGGCACCTCAGGCTCCCTGGTCATCAGTGGGCTCCAGTCTGAAGACG
AGGCTGATTACTACTGTGCGTCATGGGATGACAGTCTGAATGGTCGGGTG
TTCGGCGGAGGGACCAAGCTGACCGTCCTAG (SEQ ID NO :238)

>CB2R2P4 C3 VL amino acid sequence
QSVLTQPPSASGTPGQRVTISCSGSSSNIGSNTVNWYQRLPGAAPQLLIY
NNDQRPSGIPDRFSGSKSGTSGSLVISGLQSEDEADYYCASWDDSLNGRV
FGGGTKLTVL (SEQ ID NO :239)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the C3 antibody have the following sequences: TSGMSVI (SEQ ID NO: 240); RIDSDDEKHYNTSLKT (SEQ ID NO: 241); and LRAGSGPYVFDS (SEQ ID NO: 242). The light chain CDRs of the C3 antibody have the following sequences: SGSSSNIGSNTVN (SEQ ID NO: 243); NNDQRPS (SEQ ID▫NO:▫244);▫and ASWDDSLNGRV (SEQ ID NO: 245).

An exemplary huIP-10 monoclonal antibody is the CB21R3P1_F1 (“CB_F1”) antibody described herein. As shown below, the CB_F1 antibody includes a heavy chain variable region (SEQ ID NO: 247) encoded by the nucleic acid sequence shown in SEQ ID NO: 246, and a light chain variable region (SEQ ID NO: 339) encoded by the nucleic acid sequence shown in SEQ ID NO: 338.

>CB21R3P1_F1 VH nucleic acid sequence
CAGGTGCAGCTGGTGGAGTCTGGGGGAGGCGTGGTCCGGCCTGGGAGGTC
CCTGAGACTCTCCTGTGCAGCCTCTGGATTCACCTTCAGTAGCTATGGCA
TGCACTGGGTCCGCCAGGCTCCAGGCAAGGGGCTGGAGTGGGTGGCAGTT
ATATCATATGATGGAAGTATTAAATACTATGCAGACTCCGTGAAGGGCCG
ATTCACCATCTCCAGAGACAATTCCAAGAACACGCTGTATCTGCAAATGA
ACAGCCTGAGAGCTGAGGACACGGCCATTTATTACTGTGCGAGAGTGATG
GGGACGGATCCCCACTCCTACTACTACATGGACGTCTGGGGGAAGGGGAC
CCTGGTCACCGTCTCGAGT (SEQ ID NO :246)

>CB21R3P1_F1 VH amino acid sequence
QVQLVESGGGVVRPGRSLRLSCAASGFTFSSYGMHWVRQAPGKGLEWVAV
ISYDGSIKYYADSVKGRFTISRDNSKNTLYLQMNSLRAEDTAIYYCARVM
GTDPHSYYYMDVWGKGTLVTVSS (SEQ ID NO : 247)

>CB21R3P1_F1 VL nucleic acid sequence
TCTTCTGAGCTGACTCAGGACCCTGCTGTGTCTGTGGCCTTGGGACAGAC
AGTCAGGATCACATGCCAAGGAGACAGCCTCAGAAGCTATTATGCAAGCT
GGTACCAGCGGAAGCCAGGACAGGCCCCTGTACTTGTCATCTATGGTAAA
AACAACCGGCCCTCAGGGATCCCAGACCGATTCTCTGGCTCCAGCTCAGG
AAACACAGCTTCCTTGACCATCACTGGGGCTCAGGCGGAAGATGAGGCTG
ACTATTACTGTAACTCCCGGGACAGCAGTGGTAACCATGTGCTTTTCGGC
GGAGGGACCAAGCTGACCGTCCTA (SEQ ID NO :338)

>CB21R3P1_F1 VL amino acid sequence
SSELTQDPAVSVALGQTVRITCQGDSLRSYYASWYQRKPGQAPVLVIYGK
NNRPSGIPDRFSGSSSGNTASLTITGAQAEDEADYYCNSRDSSGNHVLFG
GGTKLTVL (SEQ ID NO :339)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the CB_F1 antibody have the following sequences: SYGMH (SEQ ID NO: 230); VISYDGSIKYYADSVKG (SEQ ID NO: 231); and VMGTDPHSYYYMDV (SEQ ID NO: 232). The light chain CDRs of the CB_F1 antibody have the following sequences: QGDSLRSYYAS (SEQ ID NO: 233); GKNNRPS (SEQ ID NO: 234); and NSRDSSGNHVL (SEQ ID NO: 235).

An exemplary huIP-10 monoclonal antibody is the CB21R3P3_E5 (“E5”) antibody described herein. As shown below, the E5 antibody includes a heavy chain variable region (SEQ ID NO: 250) encoded by the nucleic acid sequence shown in SEQ ID NO: 249, and a light chain variable region (SEQ ID NO: 252) encoded by the nucleic acid sequence shown in SEQ ID NO: 251.

>CB21R3P3_E5 VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAGCTATGGCA
TGCACTGGGTCCGCCAGGCTCCAGGCAAGGGGCTGGAGTGGGTGGCAGTT
ATATCATATGATGGAAGTATTAAATACTATGCAGACTCCGTGAAGGGCCG
ATTCACCATCTCCAGAGACAATTCCAAGAACACGCTGTATCTGCAAATGA
ACAGCCTGAGAGCTGAGGACGCGGCTGTGTATTACTGTGCGAGAGCACCA
GATGGCCACCAACTTGACTACTGGGGCAGGGGCACCCTGGTCACCGTCTC
GAGT (SEQ ID NO :249)

>CB21R3P3_E5 VH amino acid sequence
QVQLVESGGGVVQPGRSLRLSCAASGFTFSSYGMHWVRQAPGKGLEWVAV
ISYDGSIKYYADSVKGRFTISRDNSKNTLYLQMNSLRAEDAAVYYCARAP
DGHQLDYWGRGTLVTVSS (SEQ ID NO :250)

>CB21R3P3_E5 VL nucleic acid sequence
AATTTTATGCTGACTCAGCCCCACTCTGTGTCGGAGTCTCCGGGGAAGAC
GGTAACCATCTCCTGCACCGGCAGCAGTGGCAGCATTGCCAGCAACTATG
TGCAGTGGTACCAGCAGCGCCCGGGCAGTGCCCCCACCACTGTGATCTAT
GAAGATGACCAAAGACCCTCTGACGTCCCTGATCGCTTCTCTGGCTCCAT
CGACAGCTCCTCCAACTCTGCCTCCCTCACCATCTCTGGACTGAGGACTG
AGGACGAGGCTGACTACTACTGTCAGTCTTATGTTAGCAGCAAGTGGGTG
TTCGGCGGAGGGACCAAGCTGACCGTCCTA (SEQ ID NO :251)

>CB21R3P3_E5 VL amino acid sequence
NFMLTQPHSVSESPGKTVTISCTGSSGSIASNYVQWYQQRPGSAPTTVIY
EDDQRPSDVPDRFSGSIDSSSNSASLTISGLRTEDEADYYCQSYVSSKWV
FGGGTKLTVL (SEQ ID NO :252)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the E5 antibody have the following sequences: SYGMH (SEQ ID NO: 230); VISYDGSIKYYADSVKG (SEQ ID NO: 231); and APDGHQLDY (SEQ ID NO: 253). The light chain CDRs of the E5 antibody have the following sequences: TGSSGSIASNYVQ (SEQ ID NO: 327); EDDQRPS (SEQ ID NO: 254); and QSYVSSKWV (SEQ ID NO: 255).

An exemplary huIP-10 monoclonal antibody is the CB21R3P6_G7 (“G7”) antibody described herein. As shown below, the G7 antibody includes a heavy chain variable region (SEQ ID NO: 257) encoded by the nucleic acid sequence shown in SEQ ID NO: 256, and a light chain variable region (SEQ ID NO: 259) encoded by the nucleic acid sequence shown in SEQ ID NO: 258.

>CB21R3P6_G7 VH nucleic acid sequence
CAGGTGCAGCTGGTGCAGTCTGGGGCTGAGGTGAAGAAGCCTGGGTCCTC
GGTGACGGTCTCCTGCAAGGCCTCTGGAGGCACCTTCAGCAGCTTTTCTA
TCACCTGGCTGCGACAGGCCCCTGGACAAGGGCTTGAGTGGATGGGAGAG
ATCACCCCTATGTTTGGTATAGCAAACTACGCACAGAAGTTCCAGGGTAG
GGTCACGATTAGCGCGGACGAGTCCACGAGCACAGCCTACATGGAGTTGA
GTGGCCTGACATCTGAAGACACGGCCATGTATTATTGTGCGAGAGATGGT
CGGTTTGATGTTTCCGATCTTTTGACTGACAAACCCAAAGTAACGATAAA
CTACAACGGGATGGACGTCTGGGGCCAAGGCACCCTGGTCACCGTCTCGA
GT (SEQ ID NO :256)

>CB21R3P6_G7 VH amino acid sequence
QVQLVQSGAEVKKPGSSVTVSCKASGGTFSSFSITWLRQAPGQGLEWMGE
ITPMFGIANYAQKFQGRVTISADESTSTAYMELSGLTSEDTAMYYCARDG
RFDVSDLLTDKPKVTINYNGMDVWGQGTLVTVSS (SEQ ID NO :257)

>CB21R3P6_G7 VL nucleic acid sequence
CAGTCTGTGCTGACTCAGCCACCCTCAGCGTCTGGGACCCCCGGGCAGAG
GGTCACCATCTCTTGTTCTGGAAGCAGCTCCAACATCGGGAGTAACACTG
TAAACTGGTACCAGCGACTCCCAGGAGCGGCCCCCCAACTCCTCATCTAC
AATAATGACCAGCGGCCCTCAGGGATCCCTGACCGATTCTCTGGCTCCAA
GTCTGGCACCTCAGGCTCCCTGGTCATCAGTGGGCTCCAGTCTGAAGATG
AGGCTGATTACTACTGTGCGTCATGGGATGACAGTCTGAATGGTCGGGTG
TTCGGCGGAGGGACCAAGCTGACCGTCCTA (SEQ ID NO :258)

>CB21R3P6_G7 VL amino acid sequence
QSVLTQPPSASGTPGQRVTISCSGSSSNIGSNTVNWYQRLPGAAPQLLIY
NNDQRPSGIPDRFSGSKSGTSGSLVISGLQSEDEADYYCASWDDSLNGRV
FGGGTKLTVL (SEQ ID NO :259)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the G7 antibody have the following sequences: SFSIT (SEQ ID NO: 260); EITPMFGIANYAQKFQG (SEQ ID NO: 261); and DGRFDVSDLLTDKPKVTINYNGMDV (SEQ ID NO: 262). The light chain CDRs of the G7 antibody have the following sequences: SGSSSNIGSNTVN (SEQ ID NO: 243); NNDQRPS (SEQ ID NO: 244); and ASWDDSLNGRV (SEQ ID NO: 245).

An exemplary huIP-10 monoclonal antibody is the CC21R3P1_A2 (“A2”) antibody described herein. As shown below, the A2 antibody includes a heavy chain variable region (SEQ ID NO: 264) encoded by the nucleic acid sequence shown in SEQ ID NO: 263, and a light chain variable region (SEQ ID NO: 266) encoded by the nucleic acid sequence shown in SEQ ID NO: 265.

>CC21R3P1_A2 VH nucleic acid sequence
CAGCTGGTGGAGTCTGGAGGAGGCTTGATCCAGCCTGGGGGGTCCCTGAG
ACTTTCCTGTGCAGCCTCTGGATTCAGCGTCAGTGACACCTACATGAACT
GGGTCCGCCAGGCTCCAGGGAAGGGCCTGGAGTGGGTGTCAAGTATTTAT
AGCGATGATAGCACATACTACGCAGACTCCGTGAAGGGCAGATTCACCAT
CTCCAGAGACAATTCCAAGAACACGCTGTTTCTTCAAATAAACAGTTTGA
GAGCCGAGGACACGGCTGTCTATTACTGTGCGAGAGATAAGGAGTATGTA
ACATCAACTGGGGGCGCCTACTACTACTTCTACTACATGGACGTCTGGGG
CCAGGGCACCCTGGTCACCGTCTCGAGT (SEQ ID NO :263)

>CC21R3P1A2 VH amino acid sequence
QLVESGGGLIQPGGSLRLSCAASGFSVSDTYMNWVRQAPGKGLEWVSSIY
SDDSTYYADSVKGRFTISRDNSKNTLFLQINSLRAEDTAVYYCARDKEYV
TSTGGAYYYFYYMDVWGQGTLVTVSS (SEQ ID NO :264)

>CC21R3P1A2 VL nucleic acid sequence
CAGTCTGTGCTGACTCAGCCACCCTCAGCGTCTGGGACCCCCGGGCAGAG
AGTCTCCATCTCCTGTTCTGGAAGCAGCTCCAACATCGGAAGTGATACTG
TGAACTGGTACCAGCACCTCCCAGGAACGGCCCCCAAACTCCTCATCTAT
AATAATAATCAGCGGCCCTCAGGGGTCCCTGACCGATTCTCTGGCTCCAA
GTCTGGCACCTCAGCCTCCCTGGCCATCAGTGGGCTCCAGTCTGAGGATG
AGGCTGATTATTACTGTGCCGCATGGGATGACAGCCTGAATGGTCTGGTA
TTCGGCGGAGGGACCAAGGTCACCGTCCTA (SEQ ID NO :265)

>CC21R3P1A2 VL amino acid sequence
QSVLTQPPSASGTPGQRVSISCSGSSSNIGSDTVNWYQHLPGTAPKLLIY
NNNQRPSGVPDRFSGSKSGTSASLAISGLQSEDEADYYCAAWDDSLNGLV
FGGGTKVTVL (SEQ ID NO :266)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the A2 antibody have the following sequences: DTYMN (SEQ ID NO: 323); SIYSDDSTYYADSVKG (SEQ ID NO: 324); and DKEYVTSTGGAYYYFYYMDV (SEQ ID NO: 325). The light chain CDRs of the A2 antibody have the following sequences: SGSSSNIGSDTVN (SEQ ID NO: 267); NNNQRPS (SEQ ID NO: 268); and AAWDDSLNGLV (SEQ ID NO: 269).

An exemplary huIP-10 monoclonal antibody is the CC21R3P1_C1A (“C1a”) antibody described herein. As shown below, the C1a antibody includes a heavy chain variable region (SEQ ID NO: 271) encoded by the nucleic acid sequence shown in SEQ ID NO: 270, and a light chain variable region (SEQ ID NO: 273) encoded by the nucleic acid sequence shown in SEQ ID NO: 272.

>CC21R3P1C1A VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAGCTATGGCA
TGCACTGGGTCCGCCAGGCTCCAGGCAAGGGGCTGGAGTGGGTGGCAGTT
ATATCATATGATGGAAGTAATAAATACTATGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAAAGACGAG
TTTGATGCTTTTGATATCTGGGGCCGAGGGACAATGGTCACCGTCTCGAG
T (SEQ ID NO :270)

>CC21R3P1C1A VH amino acid sequence
QVQLVESGGGVVQPGRSLRLSCAASGFTFSSYGMHWVRQAPGKGLEWVAV
ISYDGSNKYYADSVKGRFTISRDNSKNTLYLQMNSLRAEDTAVYYCAKDE
FDAFDIWGRGTMVTVSS (SEQ ID NO :271)

>CC21R3P1C1A VL nucleic acid sequence
TCCTATGTGCTGACTCAGCCCCCCTCAGTGTCGGTGGCCCCAGGAACGAC
GGCCAGGATTACCTGTGGGGGAAACAATATCGGAAGTAGGAGTGTGCATT
GGTACCAGCAGAAGCCAGGCCAGGCCCCTCTACTGGTCATCTATTATGAT
AGTGACCGGCCCTCAGGGATCCCTCTGCGATTCTCTGGCTCCAACTCTGG
AAACACGGCCACCCTGACCATCAGTAGGGTCGAAGCCGGGGATGAGGCCG
ACTATTACTGTCAGGTGTGGGATACTAGTAGTGGTCATTATGTCTTCGGA
ACTGGGACCAAGGTCACCGTCCTA (SEQ ID NO :272)

> CC21R3P1_C1A VL amino acid sequence
SYVLTQPPSVSVAPGTTARITCGGNNIGSRSVHWYQQKPGQAPLLVIYYD
SDRPSGIPLRFSGSNSGNTATLTISRVEAGDEADYYCQVWDTSSGHYVFG
TGTKVTVL (SEQ ID NO :273)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the C1a antibody have the following sequences: SYGMH (SEQ ID NO: 230); VISYDGSNKYYADSVKG (SEQ ID NO: 202); and DEFDAFDI (SEQ ID NO: 274). The light chain CDRs of the C1a antibody have the following sequences: GGNNIGSRSVH (SEQ ID NO: 275); YDSDRPS (SEQ ID NO: 276); and QVWDTSSGHYV (SEQ ID NO: 277).

An exemplary huIP-10 monoclonal antibody is the CC21R3P3_C1 (“C1”) antibody described herein. As shown below, the C1 antibody includes a heavy chain variable region (SEQ ID NO: 298) encoded by the nucleic acid sequence shown in SEQ ID NO: 297, and a light chain variable region (SEQ ID NO: 300) encoded by the nucleic acid sequence shown in SEQ ID NO: 299.

>CC21R3P3_C1 VH nucleic acid sequence
CAGGTGCAGCTGGTGGAGTCTGGGGGAGGCGTGGTCCAGCCTGGGAGGTC
CCTGAGACTCTCCTGTGCAGCCTCTGGATTCACCTTCAGTAGCTATGGCA
TGCACTGGGTCCGCCAGGCTCCAGGCAAGGGGCTGGAGTGGGTGGCAGTT
ATATCATATGATGGAAGTATTAAATACTATGCAGACTCCGTGAAGGGCCG
ATTCACCATCTCCAGAGACAATTCCAGGAACACGCTGTATCTGCAGATGA
ACAGCCTGAGACCTGAGGACACGGCTGTTTATTACTGTGCGAAAGATTGG
GGATTTAGCGGCTCCCTAACATTTGATTATTGGGGCCAAGGGACAATGGT
CACCGTCTCGAGT (SEQ ID NO :297)

>CC21R3P3_C1 VH amino acid sequence
QVQLVESGGGVVQPGRSLRLSCAASGFTFSSYGMHWVRQAPGKGLEWVAV
ISYDGSIKYYADSVKGRFTISRDNSRNTLYLQMNSLRPEDTAVYYCAKDW
GFSGSLTFDYWGQGTMVTVSS (SEQ ID NO :298)

>CC21R3P3C1 VL nucleic acid sequence
TCCTATGTGCTGACTCAGCCACCCTCAGTGTCAGTGGCCCCAGGAAAGAC
GGCCAGGATTACCTGTGGGGGAAACAACATTGGAAGTAAAAGTGTGCACT
GGTACCAGCAGAAGCCAGGCCAGGCCCCTGTGCTGGTCATCTATTATGAT
AGCGACCGGCCCTCAGGGATCCCTGAGCGATTCTCTGGCTCCAACTCTGG
GAACACGGCCACCCTGACCATCAGCAGGGTCGAAGCCGGGGATGAGGCCG
ACTATTACTGTCAGGTGTGGGATAGTAGTAGTGATCATGTGGTATTCGGC
GGAGGGACCAAGGTCACCGTCCTA (SEQ ID NO :299)

>CC21R3P3_C1 VL amino acid sequence
SYVLTQPPSVSVAPGKTARITCGGNNIGSKSVHWYQQKPGQAPVLVIYYD
SDRPSGIPERFSGSNSGNTATLTISRVEAGDEADYYCQVWDSSSDHWFGG
GTKVTVL (SEQ ID NO :300)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the C1 antibody have the following sequences: SYGMH (SEQ ID NO: 230); VISYDGSIKYYADSVKG (SEQ ID NO: 231); and DWGFSGSLTF (SEQ ID NO: 301). The light chain CDRs of the C1 antibody have the following sequences: GGNNIGSKSVH (SEQ ID NO: 302); YDSDRPS (SEQ ID NO: 276); and QVWDSSSDHVV (SEQ ID NO: 303).

An exemplary huIP-10 monoclonal antibody is the CC21R3P1_E7 (“E7”) antibody described herein. As shown below, the E7 antibody includes a heavy chain variable region (SEQ ID NO: 279) encoded by the nucleic acid sequence shown in SEQ ID NO: 278, and a light chain variable region (SEQ ID NO: 281) encoded by the nucleic acid sequence shown in SEQ ID NO: 280.

>CC21R3P1E7 VH nucleic acid sequence
CAGCTGGTGGAGTCTGGGGGAGGCGTGGTCCAGCCTGGGAGGTCCCTGAG
ACTCTCCTGTGCAGCCTCTGGATTCACCTTCAGTACCTATGGCATGCACT
GGGTCCGCCAGGCTCCAGGCAAGGGGCTGGAGTGGGTGGCAGTTATATCA
TATGATGGAGGTACTAAATACTATGCAGACTCCGTGAAGGGCCGATTCAC
CATCTCCAGAGACAATTCCATGAAAACGCTCTATCTGCAAATGAACAGCC
TGAGAACTGAGGACACGGCTGTGTATTACTGTGCGAAAGATCTGGGGGAC
CTACCCCCGGGCCTTGACTACTGGGGCCAGGGGACAATGGTCACCGTCTC
GAGT (SEQ ID NO :278)

>CC21R3P1E7 VH amino acid sequence
QLVESGGGVVQPGRSLRLSCAASGFTFSTYGMHWVRQAPGKGLEWVAVIS
YDGGTKYYADSVKGRFTISRDNSMKTLYLQMNSLRTEDTAVYYCAKDLGD
LPPGLDYWGQGTMVTVSS (SEQ ID NO :279)

>CC21R3P1E7 VL nucleic acid sequence
CAGTCTGTGCTGACTCAGCCACCCTCAGCGTCTGGGACCCCCGGGCAGAG
GGTCACCATCTCTTGTTCTGGAAGCAGCTCCAACATCGGAAGTAATACTG
TAAACTGGTACCAGCAGCTCCCAGGAGCGGCCCCCAAACTCCTCATCTAT
ACTAATAATCAGCGGCCCTCAGGGGTCCCTGACCGATTCTCTGGCTCCAA
GTCTGGCACCTCAGCCTCCCTGGCCATCAGTGGGCTCCAGTCTGAGGATG
AGGCTGATTATTACTGTGCAGCATGGGATGACAGCCTGAATGGTAATGTG
GTATTCGGCGGAGGGACCAAGGTCACCGTCCTA (SEQ ID NO :280)

>CC21R3P1E7 VL amino acid sequence
QSVLTQPPSASGTPGQRVTISCSGSSSNIGSNTVNWYQQLPGAAPKLLIY
TNNQRPSGVPDRFSGSKSGTSASLAISGLQSEDEADYYCAAWDDSLNGNV
VFGGGTKVTVL (SEQ ID NO :281)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the E7 antibody have the following sequences: TYGMH (SEQ ID NO: 282); VISYDGGTKYYADSVKG (SEQ ID NO: 283); and DLGDLPPGLDY (SEQ ID NO: 284). The light chain CDRs of the E7 antibody have the following sequences: SGSSSNIGSNTVN (SEQ ID NO: 243); TNNQRPS (SEQ ID NO: 285); and AAWDDSLNGNVV (SEQ ID NO: 286).

An exemplary huIP-10 monoclonal antibody is the CC21R3P1_H6 (“H6”) antibody described herein. As shown below, the H6 antibody includes a heavy chain variable region (SEQ ID NO: 288) encoded by the nucleic acid sequence shown in SEQ ID NO: 287, and a light chain variable region (SEQ ID NO: 290) encoded by the nucleic acid sequence shown in SEQ ID NO: 289.

>CC21R3P1H6 VH nucleic acid sequence
CAGGTGCAGCTGGTGGAGTCTGGGGGAGGCGTGGCTCAGTCTGGGAAGTC
CCTGAGACTCTCCTGTGCAGCCTCTGGATTCACCTTCAGTAACTATGGCA
TGCACTGGGTCCGCCAGGCTCCAGGCAAGGGGCTGGAGTGGCTGGCAGTC
ATATCATATGATGGAAGTAACAGATACTATGCAGACTCCGTGAAGGGCCG
ATTCACCATCTCCAGAGACAATTCCAACAACACACTGAATCTGCAAATGA
GCAGCCTGAGAGCTGAGGACACGGCTCTATATTACTGTGCGAAAGATGCC
GGGGGGCCGCTTGATTACTGGGGCAAGGGCACCCTGGTCACCGTCTCGAG
T (SEQ ID NO : 287)

>CC21R3P1H6 VH amino acid sequence
QVQLVESGGGVAQSGKSLRLSCAASGFTFSNYGMHWVRQAPGKGLEWLAV
ISYDGSNRYYADSVKGRFTISRDNSNNTLNLQMSSLRAEDTALYYCAKDA
GGPLDYWGKGTLVTVSS (SEQ ID NO :288)

>CC21R3P1H6 VL nucleic acid sequence
CAGTCTGTGCTGACTCAGCTGACTCAGCCACCCTCGGTGTCACTGGCCCC
AGGACAGACGGCCACCATTACTTGTGGGGGAGACAACATTGGACGTAAAA
GTGTGCACTGGTACCAGCAGAAGCCAGGCCAGGCCCCTGTGTTGGTCGTC
TATGATGACACCGACCGGCCCTCAGGGATCCCTGAGCGATTCTCTGGCTC
CAACTCTGGGAACACGGCCACCCTAACCATCAGCAGGGTCGAAGCCGGGG
ATGAGGCCGACTATTACTGTCAGGTGTGGGATAGTAGTATTGATCATTCT
TGGGTGTTCGGCGGAGGGACCAAGCTGACCGTCCTAG (SEQ ID NO :
289)

>CC21R3P1H6 VL amino acid sequence
QSVLTQLTQPPSVSLAPGQTATITCGGDNIGRKSVHWYQQKPGQAPVLVV
YDDTDRPSGIPERFSGSNSGNTATLTISRVEAGDEADYYCQVWDSSIDHS
WVFGGGTKLTVL (SEQ ID NO :290)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the H6 antibody have the following sequences: NYGMH (SEQ ID NO: 291); VISYDGSNRYYADSVKG (SEQ ID NO: 292); and DAGGPLDY (SEQ ID NO: 293). The light chain CDRs of the H6 antibody have the following sequences: GGDNIGRKSVH (SEQ ID NO: 294); DDTDRPS (SEQ ID NO: 295); and QVWDSSIDHSWV (SEQ ID NO: 296).

An exemplary huIP-10 monoclonal antibody is the CC21R3P4_F4 (“F4”) antibody described herein. As shown below, the F4 antibody includes a heavy chain variable region (SEQ ID NO: 305) encoded by the nucleic acid sequence shown in SEQ ID NO: 304, and a light chain variable region (SEQ ID NO: 307) encoded by the nucleic acid sequence shown in SEQ ID NO: 306.

>CC21R3P4_F4 VH nucleic acid sequence
CAGGTGCAGCTGGTGGAGTCTGGGGGAGGCGTGGCTCACTCTGGGAAGTC
CCTGAGACTCTCCTGTGCAGCCTCTGGATTCACCTTCAGTAACTATGGCA
TGCACTGGGTCCGCCAGGCTCCAGGCAAGGGGCTGGAGTGGCTGGCAGTC
ATATCATATGATGGGAGTAATAGATACTATGCAGACTCCGTGAAGGGCCG
ATTCACCATCTCCAGAGACAATTCCAACAACACGCTGAATCTGCAAATGA
GCAGCCTGAGAGCTGAGGACACGGCTCTGTATTACTGTGCGAAAGATGCC
GGGGGGCCGCTTGATTACTGGGGCCGGGGCACCCTGGTCACCGTCTCGAG
T (SEQ ID NO : 304)

>CC21R3P4F4 VH amino acid sequence
QVQLVESGGGVAHSGKSLRLSCAASGFTFSNYGMHWVRQAPGKGLEWLAV
ISYDGSNRYYADSVKGRFTISRDNSNNTLNLQMSSLRAEDTALYYCAKDA
GGPLDYWGRGTLVTVSS (SEQ ID NO : 305)

>CC21R3P4F4 VL nucleic acid sequence
TCCTATGTGCTGACTCAGCCACCCTCGGTGTCAGTGGCCCCAGGACAGAT
GGCCAGAATTACCTGTGGGGGAAACAACATTGGAGATAAAAGTGTGCAAT
GGTACCAGCAGAGGCCAGGCCAGGCCCCTCTACTGGTCGTCTATGATGAT
AGCGACCGGCCCTCAGGGATCCCTGAGCGCTTCTCTGGCTCCTACTCTAG
GAACACGGCCACCCTGACCATCAGCAGGGTCGAAGCCGGGGATGAGGCCG
ACTATTACTGTCAGGTGTGGGATAGTAGTAGTGATCATCCGGAGGTGGTT
TTCGGCGGAGGGACCAAGCTGACCGTCCTA (SEQ ID NO : 306)

>CC21R3P4F4 VL amino acid sequence
SYVLTQPPSVSVAPGQMARITCGGNNIGDKSVQWYQQRPGQAPLLVVYDD
SDRPSGIPERFSGSYSRNTATLTISRVEAGDEADYYCQVWDSSSDHPEWF
GGGTKLTVL (SEQ ID NO : 307)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the F4 antibody have the following sequences: NYGMH (SEQ ID NO: 291); VISYDGSNRYYADSVKG (SEQ ID NO: 292); and DAGGPLDY (SEQ ID NO: 293). The light chain CDRs of the F4 antibody have the following sequences: GGNNIGDKSVQ (SEQ ID NO: 308); DDSDRPS (SEQ ID NO: 309); and QVWDSSSDHPEVV (SEQ ID NO: 310).

An exemplary huIP-10 monoclonal antibody is the CC21R3P5_C5 (“C5”) antibody described herein. As shown below, the C5 antibody includes a heavy chain variable region (SEQ ID NO: 312) encoded by the nucleic acid sequence shown in SEQ ID NO: 311, and a light chain variable region (SEQ ID NO: 314) encoded by the nucleic acid sequence shown in SEQ ID NO: 313.

>CC21R3P5_C5 VH nucleic acid sequence
CAGGTCCAGCTGGTGCAGTCTGGGGGAGGCGTGGTCCAGCCTGGGAGGTC
CCTGAGACTCTCCTGTGCAGCCTCTGGATTCACCTTCAGTACCTATGGCA
TGCACTGGGTCCGCCAGGCTCCAGGCAAGGGGCTGGAGTGGGTGGCAGTT
ATATCATATGATGGAGGTACTAAATACTATGCAGACTCCGTGAAGGGCCG
ATTCACCATCTCCAGAGACAATTCCATGAAAACGCTCTATCTGCAAATGA
ACAGCCTGAGAACTGAGGACACGGCTGTGTATTACTGTGCGAAAGATCTG
GGGGACCTACCCCCGGGCCTTGACTACTGGGGCCGAGGGACAATGGTCAC
CGTCTCGAGT (SEQ ID NO : 311)

>CC21R3P5C5 VH amino acid sequence
QVQLVQSGGGVVQPGRSLRLSCAASGFTFSTYGMHWVRQAPGKGLEWVAV
ISYDGGTKYYADSVKGRFTISRDNSMKTLYLQMNSLRTEDTAVYYCAKDL
GDLPPGLDYWGRGTMVTVSS (SEQ ID NO : 312)

>CC21R3P5C5 VL nucleic acid sequence
TCCTATGAGCTGACTCAGCCACCCTCAGTGTCAGTGGCCCTGGGACAGAC
GGCCACGATTACCTGTGGGGGGAGCAGTATTGAGAGTAAAAGTGTACACT
GGTACCAGGAGAAGCCAGGCCAGGCCCCTGTCCTGGTCATCTATAAAGAT
TCCAACCGGCCCTCTGTGATCCCTGAGCGATTCTCTGGCTCCAACTCGGG
GAACACGGCCACCCTGACCATCGGCAGAGCCCAAGCCGGGGATGAGGCTG
ACTATTACTGTCAGGTGTGGGACAGCAGTACTGGTGTGGTATTCGGCGGA
GGGACCAAGCTGACCGTCCTA (SEQ ID NO : 313)

>CC21R3P5C5 VL amino acid sequence
SYELTQPPSVSVALGQTATITCGGSSIESKSVHWYQEKPGQAPVLVIYKD
SNRPSVIPERESGSNSGNTATLTIGRAQAGDEADYYCQVWDSSTGVVFGG
GTKLTVL (SEQ ID NO : 314)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the C5 antibody have the following sequences: TYGMH (SEQ ID NO: 282); VISYDGGTKYYADSVKG (SEQ ID NO: 283); and DLGDLPPGLDY (SEQ ID NO: 284). The light chain CDRs of the C5 antibody have the following sequences: GGSSIESKSVH (SEQ ID NO: 315); KDSNRPS (SEQ ID NO: 316); and QVWDSSTGVV (SEQ ID NO: 317).

An exemplary huIP-10 monoclonal antibody is the CE7C1R3H8_J9 (“J9”) antibody described herein. As shown below, the J9 antibody includes a heavy chain variable region (SEQ ID NO: 319) encoded by the nucleic acid sequence shown in SEQ ID NO: 318, and a light chain variable region (SEQ ID NO: 321) encoded by the nucleic acid sequence shown in SEQ ID NO: 320.

>CE7C1R3H8_J9 VH nucleic acid sequence
CAGGTCCAGTTGGTGGAGTCTGGGGGAGGCGTGGTCCAGCCTGGGAGGTC
CCTGAGACTCTCCTGTGCAGCCTCTGGATTCACCTTCAGTACCTATGGCA
TGCACTGGGTCCGCCAGGCTCCAGGCAAGGGGCTGGAGTGGGTGGCAGTT
ATATCATATGATGGAGGTACTAAATACTATGCAGACTCCGTGAAGGGCCG
ATTCACCATCTCCAGAGACAATTCCATGAAAACGCTCTATCTGCAAATGA
ACAGCCTGAGAACTGAGGACACGGCTGTGTATTACTGTGCGAAAGATCTG
GGGGACCTACCCCCGGGCCTTGACTACTGGGGCCAGGGGACAATGGTCAC
CGTCTCGAGT (SEQ ID NO :318)

>CE7C1R3H8J9 VH amino acid sequence
QVQLVESGGGVVQPGRSLRLSCAASGFTFSTYGMHWVRQAPGKGLEWVAV
ISYDGGTKYYADSVKGRFTISRDNSMKTLYLQMNSLRTEDTAVYYCAKDL
GDLPPGLDYWGQGTMVTVSS (SEQ ID NO :319)

>CE7C1R3H8J9 VL nucleic acid sequence
CAGTCTGTGCTGACTCAGCCACCCTCAGCGTCTGGGACCCCCGGGCAGAG
GGTCACCATCTCTTGTTCTGGAAGCAGCTCCAACATCGGAAGTAATACTG
TAAACTGGTACCAGCAGCTCCCAGGAGCGGCCCCCAAACTCCTCATCTAT
ACTAATAATCAGCGGCCCTCAGGGGTCCCCGACCGATTCTCTGGCTCCAA
GTCTGGCACCTCAGCCTCCCTGGCCATCAGTGGGCTCCAGTCTGAGGATG
AGGCTGATTATTACTGTGCAGCATGGGATGACAGCTCGGAGCCTCGTGTG
GTATTCGGCGGAGGGACCAAGGTCACCGTCCTA (SEQ ID NO :320)

>CE7C1R3H8J9 VL amino acid sequence
QSVLTQPPSASGTPGQRVTISCSGSSSNIGSNTVNWYQQLPGAAPKLLIY
TNNQRPSGVPDRFSGSKSGTSASLAISGLQSEDEADYYCAAWDDSSEPRW
FGGGTKVTVL (SEQ ID NO :321)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the J9 antibody have the following sequences: TYGMH (SEQ ID NO: 282); VISYDGGTKYYADSVKG (SEQ ID NO: 283); and DLGDLPPGLDY (SEQ ID NO: 284). The light chain CDRs of the J9 antibody have the following sequences: SGSSSNIGSNTVN (SEQ ID NO: 243); TNNQRPS (SEQ ID NO: 285); and AAWDDSSEPRVV (SEQ ID NO: 322).

An exemplary huIP-10 monoclonal antibody is the CC21R3P1_B9 (“B9”) antibody described herein. As shown below, the B9 antibody includes a heavy chain variable region (SEQ ID NO: 331) encoded by the nucleic acid sequence shown in SEQ ID NO: 330, and a light chain variable region (SEQ ID NO: 333) encoded by the nucleic acid sequence shown in SEQ ID NO: 332.

>CC21R3P1B9 VH nucleic acid sequence
GAGGTGCAGCTGGTGCAGTCTGGGGGAGGCGTGGTCCAGCCTGGGAGGTC
CCTGAGACTCTCCTGTGCAGCCTCTGGATTCACCTTCAGTAGCTATGGCA
TGCACTGGGTCCGCCAGGCTCCAGGCAAGGGGCTGGAGTGGGTGGCAGTT
ATATCATATGATGGAAGTAATAAATACTATGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAAAGACGGT
GGCTGGTACGACTGGTACTTCGATCTCTGGGGCAGGGGAACCCTGGTCAC
CGTCTCGAGT (SEQ ID NO:330)

>CC21R3P1B9 VH amino acid sequence
EVQLVQSGGGVVQPGRSLRLSCAASGFTFSSYGMHWVRQAPGKGLEWVAV
ISYDGSNKYYADSVKGRFTISRDNSKNTLYLQMNSLRAEDTAVYYCAKDG
GWYDWYFDLWGRGTLVTVSS (SEQ ID NO : 331)

>CC21R3P1B9 VL nucleic acid sequence
TCTTCTGAGCTGACTCAGGACCCTGATGTGTCCGTGGCCTTGGGACAGAC
AGTCAGGATCACATGCCAAGGAGACAGCCTCACCAGCTATTATGCAAGCT
GGTACCAGCAGAAGCCAGGACAGGCCCCTGTACTTGTCATCTCTGGTAAT
GACAACCGGCCCTCAGGGATCCCAGACCGATTCTCTGGCTCCAACTCAGG
AAACACAGCTTCCTTGACCATCACTGGGGCTCAGGCGGAAGATGCGGCTG
ACTATTACTGTGGCTCCCGGGACAGCAGCGGTTACCAAGTGGTGTTCGGC
GCAGGGACCAAGCTGACCGTCCTA (SEQ ID NO: 332)

>CC21R3P1B9 VL amino acid sequence
SSELTQDPDVSVALGQTVRITCQGDSLTSYYASWYQQKPGQAPVLVISGN
DNRPSGIPDRFSGSNSGNTASLTITGAQAEDAADYYCGSRDSSGYQWFGA
GTKLTVL (SEQ ID NO : 333)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the B9 antibody have the following sequences: SYGMH (SEQ ID NO: 230); VISYDGSNKYYADSVKG (SEQ ID NO: 202); and DGGWYDWYFDL (SEQ ID NO: 334). The light chain CDRs of the B9 antibody have the following sequences: QGDSLTSYYAS (SEQ ID NO: 335); GNDNRPS (SEQ ID NO: 336); and GSRDSSGYQVV (SEQ ID NO: 337).

An exemplary huIP-10 monoclonal antibody is the CC21R3P1_F1 (“CC_F1”) antibody described herein. As shown below, the CC_F1 antibody includes a heavy chain variable region (SEQ ID NO: 222) encoded by the nucleic acid sequence shown in SEQ ID NO: 221, and a light chain variable region (SEQ ID NO: 224) encoded by the nucleic acid sequence shown in SEQ ID NO: 223.

>CC21R3P1_F1 VH nucleic acid sequence
CAGGTGCAGCTGGTGGAGTCTGGGGGAGGCGTGGTCCAGCCTGGGAGGTC
CCACACACTCTCCTGTGCAGCCTCTGGATTCGCCTTCAAAAACTCTGGCA
TACACTGGGTCCGCCAGGCTCCAGGCAAGGGACTGGAGTGGGTGGCAGTT
ATATCATATGATGGAAGTAACAAATTCTACGCAGACTCCGTGAAGGGCCG
ATTCACCATCTCCAGAGACAACTCCCAGAACACTGTATATCTGCAAATGA
CTGACCTGAGACCTGACGACACGGCTGTCTATTATTGTGCAAGAGATGGG
AGTGAGAGCGAGTACTTAGACTACTGGGGCAAGGGAACCCTGGTCACCGT
CTCGAGT (SEQ ID NO: 221)

>CC21R3P1_F1 VH amino acid sequence
QVQLVESGGGVVQPGRSHTLSCAASGFAFKNSGIHWVRQAPGKGLEWVAV
ISYDGSNKFYADSVKGRFTISRDNSQNTVYLQMTDLRPDDTAVYYCARDG
SESEYLDYWGKGTLVTVSS (SEQ ID NO : 222)

>CC21R3P1_F1 VL nucleic acid sequence
AATTTTATGCTGACTCAGCCCCACTCTGTGTCGGAGTCTCCGGGGAAGAC
GGTGACCATCTCCTGCACCGGCAGCGGTGGCAGCATTGACAGAAACTATG
TGCAGTGGTACCAGCAGCGCCCGGGCAGTGCCCCCATCACTGTGATCTAT
GAGGATAACCAAAGACCCTCTGGGGTCCCGGATCGATTCTCTGGCTCCAT
CGACAGCTCCTCCAACTCTGCCTCCCTCACCATCTCTGGACTACGGACTG
ACGACGAGGCTGACTACTACTGTCAGTCTTATGATAGCATCAATCTTTGG
GTTTTCGGCGGAGGGACCAAGGTCACCGTCCTAGG (SEQ ID NO: 22
3)

>CC21R3P1_F1 VL amino acid sequence
NFMLTQPHSVSESPGKTVTISCTGSGGSIDRNYVQWYQQRPGSAPITVIY
EDNQRPSGVPDRFSGSIDSSSNSASLTISGLRTDDEADYYCQSYDSINLW
VFGGGTKVTVL (SEQ ID NO : 224)

The amino acids encompassing the complementarity determining regions (CDR) are as defined by Chothia et al. and E.A. Kabat et al. (See Chothia, C, et al., Nature 342:877-883 (1989); Kabat, EA, et al., Sequences of Protein of immunological interest, Fifth Edition, US Department of Health and Human Services, US Government Printing Office (1991)). The heavy chain CDRs of the CC_F1 antibody have the following sequences: NSGIH (SEQ ID NO: 201); VISYDGSNKFYADSVKG (SEQ ID NO: 211); and DGSESEYLDY (SEQ ID NO: 326). The light chain CDRs of the CC_F1 antibody have the following sequences: TGSGGSIDSNYVQ (SEQ ID NO: 212); EDNQRPS (SEQ ID NO: 205); and QSYDSINLWV (SEQ ID NO: 225).

huIP-10 antibodies of the invention also include antibodies that include a heavy chain variable amino acid sequence that is at least 90%, 92%, 95%, 97%, 98%, 99% or more identical the amino acid sequence of SEQ ID NO: 198, 208, 214, 222, 227, 237, 247, 250, 257, 264, 271, 279, 288, 298, 305, 312, 319 or 331 and/or a light chain variable amino acid that is at least 90%, 92%, 95%, 97%, 98%, 99% or more identical the amino acid sequence of SEQ ID NO: 200, 210, 219, 224, 229, 239, 252, 259, 266, 273, 281, 290, 300, 307, 314, 321, 333 or 339.

The present invention provides fully human monoclonal antibodies that specifically bind interferon inducible protein 10 (IP-10, also referred to herein as CXCL10). Exemplary monoclonal antibodies include NI-0801; CF1N1R3P4_C7 (“C7”); CF1H1R3P3_G11 (“G11”); CF1H1R3P4 B5 (“B5”); CF1A11R3P3 F3 (“F3”); CC21R3P1_F1 (“CC_F1”); CB21R3P3_E5 (“E5”); CC21R3P1 H6 (“H6”); CC21R3P5_C5 (“C5”); CB1R3P4_D3 (“D3”); CB2R2P4_C3 (“C3”); CC21R3P4_F4 (“F4”); CC21R3P1_C1a (“C1a”); CC21R3P3_C1 (“C1”); CC21R3P1 E7 (“E7”); CE7C1R3H8 J9 (“J9”); CB21R3P1_F1 (“CB_F1”); CC21R3P1 A2 (“A2”); CB21R3P6_G7 (“G7”); and CC21R3P1_B9 (“B9”).

Alternatively, the monoclonal antibody is an antibody that binds to the same epitope as NI-0801, C7, G11, B5, F3, CC_F1, E5, H6, C5, D3, F4, C1a, C1, E7, J9, CB_F1, A2, G7, C3, or B9. The antibodies are respectively referred to herein as huIP-10 antibodies.

huIP-10 antibodies of the invention also include antibodies that include a heavy chain variable amino acid sequence that is at least 90%, 92%, 95%, 97%, 98%, 99% or more identical the amino acid sequence of SEQ ID NO: 198, 208, 214, 222, 227, 237, 247, 250, 257, 264, 271, 279, 288, 298, 305, 312, 319 or 331 and/or a light chain variable amino acid that is at least 90%, 92%, 95%, 97%, 98%, 99% or more identical the amino acid sequence of SEQ ID NO: 200, 210, 219, 224, 229, 239, 252, 259, 266, 273, 281, 290, 300, 307, 314, 321, 333 or 339.

Preferably, the three heavy chain complementarity determining regions (CDRs) include an amino acid sequence at least 90%, 92%, 95%, 97%, 98%, 99% or more identical to each of: (i) a VH CDR1 sequence selected from the group consisting of SEQ ID NOs: 201, 230, 240, 260, 282, 291 and 323; (ii) a VH CDR2 sequence selected from the group consisting of SEQ ID NOs: 202, 211, 231, 241, 261, 283, 292 and 324; (iii) a VH CDR3 sequence selected from the group consisting of SEQ ID NOs: 203, 217, 232, 242, 248, 253, 262, 274, 284, 293, 301, 325, 326 and 334; and a light chain with three CDR that include an amino acid sequence at least 90%, 92%, 95%, 97%, 98%, 99% or more identical to each of (iv) a VL CDR1 sequence selected from the group consisting of SEQ ID NOs: 204, 212, 233, 243, 267, 275, 294, 302, 308, 315, 327 and 335; (v) a VL CDR2 sequence selected from the group consisting of SEQ ID NOs: 205, 234, 244, 254, 268, 276, 285, 295, 309, 316 and 336; and (vi) a VL CDR3 sequence selected from the group consisting of SEQ ID NOs: 206, 220, 225, 235, 245, 255, 269, 277, 286, 296, 303, 310, 317, 322 and 337.

In one embodiment, the huIP-10 antibodies of the invention include a variable heavy chain region comprising the amino acid sequence of SEQ ID NO: 198 and a light chain variable region comprising the amino acid sequence of SEQ ID NO: 200.

The huIP-10 antibodies of the invention include a V_{H} CDR1 region comprising the amino acid sequence of SEQ ID NO: 201, a V_{H} CDR2 region comprising the amino acid sequence of SEQ ID NO: 202; and a V_{H} CDR3 region comprising the amino acid sequence of SEQ ID NO: 203. The huIP-10 antibodies include a V_{L} CDR1 region comprising the amino acid sequence of SEQ ID NO: 204; a V_{L} CDR2 region comprising the amino acid sequence of SEQ ID NO: 205; and a V_{L} CDR3 region comprising the amino acid sequence of SEQ ID NO: 206. In a preferred embodiment, the huIP-10 antibody includes a V_{H} CDR1 region comprising the amino acid sequence of SEQ ID NO: 201, a V_{H} CDR2 region comprising the amino acid sequence of SEQ ID NO: 202; a V_{H} CDR3 region comprising the amino acid sequence of SEQ ID NO: 203, a V_{L} CDR1 region comprising the amino acid sequence of SEQ ID NO: 204; a V_{L} CDR2 region comprising the amino acid sequence of SEQ ID NO: 205; and a V_{L} CDR3 region comprising the amino acid sequence of SEQ ID NO: 206.

Preferably, the huIP-10 antibodies are formatted in an IgG isotype. More preferably, the huIP-10 antibodies are formatted in an IgG1 isotype. An exemplary IgG1-formatted antibody is the IgG1-formatted NI-0801 antibody comprising the heavy chain sequence of SEQ ID NO: 329 and the light chain sequence of SEQ ID NO: 328, as shown below:

>NI-0801 Light Chain Amino Acid Sequence
NFMLTQPHSVSESPGKTVTISCTGSGGSIASNYVQWYQQRPGSSPTTVIY
EDNQRPSGVPDRFSGSIDSSSNSASLTISGLKTEDEADYYCQSYDPLPVW
VFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS (SEQ ID NO :328)

>NI-0801 Heavy Chain Amino Acid Sequence
QVQLVESGGGVVQPGRSLRLSCAASGFTFSNSGIHWVRQAPGKGLEWVAV
ISYDGSNKYYADSVKGRFTISRDNSKNTLYLQMNSLRAEDTAVYYCARLR
DNAEYTDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
(SEQ ID NO :329)

The closest germline for the huIP-10 antibodies described herein are shown below in Table 5:

TABLE 5

Closest germlines for the huIP-10 antibodies

| | | |
|---|---|---|
| Clone ID | VH dp number | VL dp number |
| NI-0801 | Vh3_DP-49_(3-30.5) | Vlambda6_6a |
| CF1N1R3P4_C7 | Vh3_DP-49_(3-30.5) | Vlambda6_6a |
| CF1H1R3P3_G11 | Vh3_DP-49_(3-30.5) | Vlambda6_6a |
| CF1A11R3P3_F3 | Vh3_DP-49_(3-30.5) | Vlambda6_6a |
| CF1H1R3P4_B5 | Vh3_DP-49_(3-30.5) | Vlambda6_6a |
| CC21R3P1_F1 | Vh3_DP-49_(3-30.5) | Vlambda6_6a |
| CC21R3P1_B9 | Vh3_DP-49_(3-30.5) | Vlambda3_DPL16_(31) |
| CB21R3P3_E5 | Vh3_DP-49_(3-30.5) | Vlambda6_6a |
| CC21R3P1_H6 | Vh3_DP-49_(3-30.5) | Vlambda3_3h |
| CC21R3P5_C5 | Vh3_DP-49_(3-30.5) | Vlambda3_3j |
| CB1R3P4_D3 | Vh3_DP-49_(3-30.5) | Vlambda3_DPL16_(31) |
| CB2R2P4_C3 | Vh2_DP-27,28_(2-70) | Vlambda1_DPL2_(1c) |
| CC21R3P4_F4 | Vh3_DP-49_(3-30.5) | Vlambda3_3h |
| CC21R3P1_C1a | Vh3_DP-49_(3-30.5) | Vlambda3_3h |
| CC21R3P3_C1 | Vh3_DP-49_(3-30.5) | Vlambda3_3h |
| CC21R3P1_E7 | Vh3_DP-49_(3-30.5) | Vlambda1_DPL2_(1c) |
| CE7C1R3H8_J9 | Vh3_DP-49_(3-30.5) | Vlambda1_DPL2_(1c) |
| CB21R3P1_F1 | Vh3_DP-49_(3-30.5) | Vlambda3_DPL16_(31) |
| CC21R3P1_A2 | Vh3_DP-86_(3-66) | Vlambda1_DPL2_(1c) |
| CB21R3P6_G7 | Vh1_DP-10_(1-69) | Vlambda1_DPL2_(1c) |

### Methods of Use

Therapeutic formulations of the invention (i.e. formulations including anti-TLR4 antibody or anti-IP-10 antibody) are used to inhibit ARDS and/or improve survival of a subject having ARDS.

Efficaciousness of treatment is determined in association with any known method for diagnosing or treating ARDS or other transplant related disorders. Inhibiting ARDS or improving survival of subject having ARDS indicates that the antibody confers a clinical benefit.

Anti-TLR4 antibodies are administered in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement : Concepts, Possibilities, Limitations, And Trends, Harwood Academic Publishers, Langhome, Pa., 1994; and Peptide And Protein Drug Delivery (Advances In Parenteral Sciences, Vol. 4), 1991, M. Dekker, New York.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods.

In some embodiments, the antibody contains a detectable label. Antibodies are polyclonal, or more preferably, monoclonal. An intact antibody, or a fragment thereof (e.g., F_{ab}, scFv, or F(_{ab})₂) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

### Exemplary Formulations of NI-0101

In the compositions and methods provided herein, the anti-TLR4 antibody is formulated as purified bulk drug substance. The potency of anti-TLR4 antibody formulation may be assessed by measuring inhibition of TLR4-mediated IL6 signaling in differentiated U937 cells. The relative potency a of a batch may be determined in comparison to a reference standard. In some embodiments, the anti-TLR4 antibody is formulated at a target concentration of 1 mg/mL to 10 mg/mL, 10 mg/mL to 20 mg/mL, 20 mg/mL to 30 mg/mL, 30 mg/mL to 40 mg/mL, 40 mg/mL to 50 mg/mL, 50 mg/mL to 60 mg/mL, 60 mg/mL to 70 mg/mL, 70 mg/mL to 80 mg/mL, 80 mg/mL to 90 mg/mL, 90 mg/mL to 100 mg/mL, 100 mg/mL to 110 mg/mL, 110 mg/mL to 120 mg/mL, 120 mg/mL to 130 mg/mL, 130 mg/mL to 140 mg/mL, 140 mg/mL to 150 mg/mL, 150 mg/mL to 160 mg/mL, 160 mg/mL to 170 mg/mL, 170 mg/mL to 180 mg/mL, 180 mg/mL to 190 mg/mL, 190 mg/mL to 200 mg/mL, 200 mg/mL to 300 mg/mL, 300 mg/mL to 400 mg/mL or 400 mg/mL to 500 mg/mL.

In some embodiments, the TLR-4 antibody is formulated at a target concentration of about 1 mg/mL, about 2 mg/mL, about 3 mg/mL, 4 mg/mL, about 5 mg/mL, about 6 mg/mL, about 7 mg/mL, about 8 mg/mL, about 9 mg/mL, 10 mg/mL, about 11 mg/mL, about 12 mg/mL, about 13 mg/mL, 14 mg/mL, about 15 mg/mL, about 16 mg/mL, about 17 mg/mL, about 18 mg/mL, about 19 mg/mL or about 20 mg/mL. In some embodiments, the anti-TLR4 antibody is formulated at a concentration of about 10 mg/mL.

In some embodiments, the TLR-4 antibody is formulated at a target concentration of about 140 mg/mL, 141 mg/mL, about 142 mg/mL, about 143 mg/mL, 144 mg/mL, about 145 mg/mL, about 146 mg/mL, about 147 mg/mL, about 148 mg/mL, about 149 mg/mL, 150 mg/mL, about 151 mg/mL, about 152 mg/mL, about 153 mg/mL, 154 mg/mL, about 155 mg/mL, about 156 mg/mL, about 157 mg/mL, about 158 mg/mL, about 159 mg/mL or about 160 mg/mL. In some embodiments, the anti-TLR4 antibody is formulated at a concentration of about 150 mg/mL.

In some embodiments, the anti-TLR4 antibody is formulated according to Table 6. In some embodiments, the anti-TLR4 antibody is formulated in about 25 mM histidine, about 200 mM sucrose and wherein the pH is about 5.8-6.2. In some embodiments, the anti-TLR4 antibody is formulated in 25 mM histidine, 200 mM sucrose and wherein the pH is 6.0. In some embodiments, the anti-TLR4 antibody is formulated as follows: about 10 mg of NI-0101, about 1.88 mg L-histidine, about 2.70 mg L-histidine monohydrochloride monohydrate, about 68.46 mg of sucrose, about 0.05 mg polysorbate 80, wherein the pH is between 5.8 and 6.2. In some embodiments, the anti-TLR4 antibody is formulated as follows: about 10 mg of NI-0101, about 1.88 mg L-histidine, about 2.70 mg L-histidine monohydrochloride monohydrate, about 68.46 mg of sucrose, about 0.05 mg polysorbate 80, and wherein the pH is 6.0.

TABLE 6

Exemplary Formulation of NI-0101

| | | |
|---|---|---|
| Ingredient | Function | Quantity (per mL) |
| NI-0101 | Active | 10 mg |
| L-Histidine | Buffer | 1.88 mg |
| L-Histidine monohydrochloride monohydrate | Buffer | 2.70 mg |
| Sucrose | Buffer | 68.46 mg |
| Polysorbate 80 | Surfactant | 0.05 mg |
| Water for Infection | | To 1 mL |

In some embodiments, the anti-TLR4 antibody is formulated according to Table 7. In some embodiments, the anti-TLR4 antibody is formulated in about 0.02% polysorbate 80 and wherein the pH is about 5.8-6.2. In some embodiments, the anti-TLR4 antibody is formulated in about 0.02% polysorbate 80 and wherein the pH is 6.0. In some embodiments, the anti-TLR4 antibody is formulated as follows: about 150 mg of NI-0101, about 5.24 mg L-histidine monohydrochloride, about 40.15 mg L-Arginine Monohydrochloride, about 1.65 mg of L-Arginine, about 0.20 mg polysorbate 80 (0.02%), wherein the pH is between 5.8 and 6.2. In some embodiments, the anti-TLR4 antibody is formulated as follows: about 150 mg of NI-0101, about 5.24 mg L-histidine monohydrochloride, about 40.15 mg L-Arginine Monohydrochloride, about 1.65 mg of L-Arginine, about 0.20 mg polysorbate 80, and wherein the pH is 6.0.

TABLE 7

Exemplary Formulation of NI-0101

| | | |
|---|---|---|
| Material | Function | Quantity (per mL) |
| NI-0101 | Active | 150 mg |
| L-Histidine monohydrochloride | Buffer | 5.24 mg |
| L-Arginine Monohydrochloride | Buffer | 40.15 mg |
| L-Arginine | Buffer | 1.65 mg |
| Polysorbate 80 (0.02%) | Surfactant | 0.20 mg |
| Water for Injection | | To 1 mL |

### Exemplary Formulation of NI-0801

In the compositions and methods provided herein, the anti-huIP-10 antibody is formulated as a sterile concentrate for infusion (per mL). In some embodiments, the anti-huIP-10 antibody is formulated as an isotonic solution. In some embodiments, the anti-huIP-10 antibody is formulated as a clear, colorless to slightly yellow solution. In some embodiments, the anti-huIP-10 antibody is formulated at a target concentration about 1 mg/mL, about 2 mg/mL, about 3 mg/mL, 4 mg/mL, about 5 mg/mL, about 6 mg/mL, about 7 mg/mL, about 8 mg/mL, about 9 mg/mL, 10 mg/mL, about 11 mg/mL, about 12 mg/mL, about 13 mg/mL, 14 mg/mL, about 15 mg/mL, about 16 mg/mL, about 17 mg/mL, about 18 mg/mL, about 19 mg/mL or about 20 mg/mL. In some embodiments, the anti-huIP-10 antibody is formulated at a concentration of about 10 mg/mL.

In some embodiments, the anti-huIP-10 antibody is formulated according to Table 8. In some embodiments, the anti-huIP-10 antibody is formulated in about 25 mM histidine hydrochloride, about 200 mM sucrose and wherein the pH is about 5.8-6.2. In some embodiments, the anti-huIP-10 antibody is formulated in 25 mM histidine hydrochloride, 200 mM sucrose and wherein the pH is 6.0. In some embodiments, the anti-huIP-10 antibody is formulated as follows: about 10 mg of NI-0801, about 1.88 mg L-histidine, about 2.70 mg L-histidine monohydrochloride monohydrate, about 68.46 mg of sucrose, wherein the pH is between 5.8 and 6.2. In some embodiments, the anti-huIP-10 antibody is formulated as follows: about 10 mg of NI-0801, about 1.88 mg L-histidine, about 2.70 mg L-histidine monohydrochloride monohydrate, about 68.46 mg of sucrose, wherein the pH is 6.0.

TABLE 8

Exemplary Formulation of NI-0801

| | | | |
|---|---|---|---|
| Ingredient | Function | Reference | Quantity (per ml) |
| NI-0801 | Active | HSE | 10 mg |
| L-Histidine | Buffer | Ph Eur | 1.88 mg |
| L-Histidine monohydrochloride monohydrate | Buffer | Ph Eur | 2.70 mg |
| Sucrose | Buffer | Ph Eur | 68.46 mg |
| Water for Injection | | Ph Eur | to 1 g |

The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In one embodiment, the active compounds are administered in combination therapy, i.e., combined with other agents, e.g., therapeutic agents, that are useful for treatment of coronavirus infection. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more antibodies of the invention can be co-formulated with, and/or co-administered with one or more additional therapeutic agents. Exemplary therapeutic agents include, but are not limited to antiviral drugs, ACE inhibitors, immune booster drugs, corticosteroids or convalescent sera or any combination thereof. Exemplary antiviral drugs include, but are not limited to Remdesivir bamlanivimab, etesevimab, casirivimab, imdevimab, a monoclonal antibody targeting the virus or any combination thereof. Exemplary ACE inhibitors include but are not limited to hydroxylchloroquine (Plaquenil) or a soluble recombinant ACE2. Exemplary immune booster drugs include an anti-TNF antibody, an anti-IP-10 antibody an anti-IL-1 antibody, anti-IL-6 monoclonal antibody such as tocilizumab (Actemra®) or sarilumab (Kevzara®), Atorvastatin (Lipitor) or Pravastatin (Pravachol). Such combination therapies may advantageously utilize lower dosages of the administered therapeutic agents, thus avoiding possible toxicities or complications associated with the various monotherapies.

Optionally, the subject is further administered with a second agent that is useful for treating a symptom associated with an autoimmune or inflammatory disorder. The second agent includes but is not limited to, an anti-cytokine or anti-chemokine reagent that recognizes cytokines such as interleukin 1 (IL-1), IL-2, IL-4, IL-6, IL-12, IL-13, IL-15, IL-17, IL-18, IL-20, IL-21, IL-22, IL-23, IL-27 and IL-31, and/or chemokines such as MIP1 alpha, MIP1 beta, RANTES, MCP1, IP-10, ITAC, MIG, SDF and fractalkine.

Preferred therapeutic agents used in combination with an antibody of the invention are those agents that interfere at different stages in a coronavirus infection. In one embodiment, one or more antibodies described herein may be coformulated with, and/or coadministered with one or more additional agents.

### Pharmaceutical Compositions

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

The active compound is administered by nasal inhalation, inhalation through the mouth, intravenously, orally, or any combination thereof. Alternatively the, active compound is administered orally via an enteric-coated capsule. In some embodiments, the antibody, formulation or pharmaceutical composition thereof (e.g. formulation or pharmaceutical composition including anti-TLR4 or anti-IP-10 is administered intravenously.

Administration by inhalation may be in the form of an inhaler or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### Dosage Regimens

The disclosure provides dosing regimens for treatment of a subject having ARDS using an anti-TLR4 antibody. In some embodiments, the anti-TLR4 antibody is administered at a dose of about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 35 mg/kg, about 40 mg/kg or about 50 mg/kg. In some embodiments, the anti-TLR4 antibody is administered at a dose of about 0.01 mg/kg to about 0.05 mg/kg. In some embodiments, the anti-TLR4 antibody is administered at a dose of about 0.05 mg/kg to about 0.1 mg/kg. In some embodiments, the anti-TLR4 antibody is administered at a dose of about 0.1 mg/kg to about 0.5 mg/kg. In some embodiments, the anti-TLR4 antibody is administered at a dose of about 0.5 mg/kg to about 1 mg/kg. In some embodiments, the anti-TLR4 antibody is administered at a dose of about 1 mg/kg to about 5 mg/kg. In some embodiments, the anti-TLR4 antibody is administered at a dose of about 5 mg/kg to about 10 mg/kg. In some embodiments, the anti-TLR4 antibody is administered at about 10 mg/kg to about 15 mg/kg. In some embodiments, the anti-TLR4 antibody is administered at a dose of about 15 mg/kg to about 20 mg/kg. In some embodiments, the anti-TLR4 antibody is administered at about 20 mg/kg to about 25 mg/kg. In some embodiments, the anti-TLR4 antibody is administered at a dose of about 15 mg/kg.

In some embodiments, the dose of the anti-TLR4 antibody is administered to a subject having ARDS over a period of about 1 hour, about 2 hours, about 3 hours, about 4 hours, about 5 hours, about 6 hours, about 7 hours, about 8 hours, about 9 hours or about 10 hours. In some embodiments, the anti-TRL4 antibody is administered to a subject having ARDS over a period of 3 hours. In some embodiments, the anti-TLR4 antibody is intravenously administered to a subject having ARDS over 3 hours.

In some embodiments, multiple doses of the anti-TLR4 is administered to a subject having ARDS. In some embodiments, the antibody is administered at least one time, at least 2 times, at least 3 times, at least 4 times, at least 5 times, at least 6 times, at least 7 times, at least 8 times, at least 9 times or at least 10 times. In some embodiments, the anti-TLR4 antibody is administered one time to the subject having ARDS.

In some embodiments, the anti-TLR4 antibody is administered as an initial dose and followed by one or more maintenance doses. In some embodiments, the one or more maintenance dose(s) is a dosage that is substantially similar to the initial dose. In some embodiments, the one or more maintenance dose(s) is a dose that is less than the initial dose. In some embodiments, the one or more maintenance dose(s) is a dose that is greater than the initial dose.

In some embodiments, the one or more maintenance dose(s) comprises at least two or more doses, wherein each maintenance dose is the same dose. In some embodiments, the two or more maintenance doses are substantially similar to the initial dose. In some embodiments, the two or more maintenance doses are greater than the initial dose. In some embodiments, the two or more maintenance doses are less than the initial dose.

In some embodiments, the one or more maintenance dose(s) comprise at least two or more doses, wherein each maintenance dose is administered in an increasing dose amount. In some embodiments, the two or more maintenance doses are administered in a decreasing dose amount.

In some embodiments, the one or more maintenance dose(s) comprise at least two or more doses, wherein each maintenance dose is administered at a periodic time interval. In some embodiments, two or more doses are administered at increasing time intervals. In some embodiments, two or more doses are administered at decreasing time intervals.

In some embodiments, a dose of the anti-TLR4 antibody is administered is administered once daily. In some embodiments, the time between each dose of anti-TLR4 antibody is about 1 day, about 2 days, about 3 days, about 4 days, about 5 days, about 6 days, about 7 days, about 8 days, about 9 days, about 10 days, about 11 days or about 12 days.

### Exemplary Anti-IP-10 Dosage Regimens

The disclosure provides dosing regimens for treatment of a subject having ARDS using an anti-IP10 antibody. In some embodiments, the anti-IP10 antibody is administered at a dose of about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 35 mg/kg, about 40 mg/kg or about 50 mg/kg. In some embodiments, the anti-IP-10 antibody is administered at a dose of about 1 mg/kg to about 5 mg/kg. In some embodiments, the anti-IP-10 antibody is administered at a dose of about 5 mg/kg to about 10 mg/kg. In some embodiments, the anti-IP-10 antibody is administered at about 10 mg/kg to about 15 mg/kg. In some embodiments, the anti-IP-10 antibody is administered at a dose of about 15 mg/kg to about 20 mg/kg. In some embodiments, the anti-IP-10 antibody is administered at about 20 mg/kg to about 25 mg/kg. In some embodiments, the anti-IP-10 antibody is administered at a dose of about 1 mg/kg. In some embodiments, the anti-IP-10 antibody is administered at a dose of about 3 mg/kg. In some embodiments, the anti-IP-10 antibody is administered at a dose of about 10 mg/kg. In some embodiments, the anti-IP-10 antibody is administered at a dose of about 15 mg/kg. In some embodiments, the anti-IP-10 antibody is administered at a dose of about 20 mg/kg.

In some embodiments, multiple doses of the anti-IP-10 is administered to a subject having ARDS. In some embodiments, the antibody is administered at least one time, at least 2 times, at least 3 times, at least 4 times, at least 5 times, at least 6 times, at least 7 times, at least 8 times, at least 9 times or at least 10 times. In some embodiments, the anti-IP-10 antibody is administered to the subject having ARDS one time.

In some embodiments, the anti-IP-10 antibody is administered as an initial dose and followed by one or more maintenance doses. In some embodiments, the one or more maintenance dose(s) is a dosage that is substantially similar to the initial dose. In some embodiments, the one or more maintenance dose(s) is a dose that is less than the initial dose. In some embodiments, the one or more maintenance dose(s) is a dose that is greater than the initial dose.

In some embodiments, the one or more maintenance dose(s) comprises at least two or more doses, wherein each maintenance dose is the same dose. In some embodiments, the two or more maintenance doses are substantially similar to the initial dose. In some embodiments, the two or more maintenance doses are greater than the initial dose. In some embodiments, the two or more maintenance doses are less than the initial dose.

In some embodiments, the one or more maintenance dose(s) comprise at least two or more doses, wherein each maintenance dose is administered in an increasing dose amount. In some embodiments, the two or more maintenance doses are administered in a decreasing dose amount.

In some embodiments, the one or more maintenance dose(s) comprise at least two or more doses, wherein each maintenance dose is administered at a periodic time interval. In some embodiments, two or more doses are administered at increasing time intervals. In some embodiments, two or more doses are administered at decreasing time intervals.

In some embodiments, a dose of the anti-IP-10 antibody is administered is administered once daily. In some embodiments, the time between each dose of anti-IP-10 antibody is about 1 day, about 2 days, about 3 days, about 4 days, about 5 days, about 6 days, about 7 days, about 8 days, about 9 days, about 10 days, about 11 days or about 12 days.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer’s specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d} > 10⁻⁶). Antibodies include, but are not limited to, polyclonal, monoclonal, chimeric, domain antibody, single chain, F_{ab}, F_{ab}, and F(_{ab′})₂ fragments, scFvs, and an F_{ab} expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989). Various methods are known in the art for numbering the amino acids sequences of antibodies and identification of the complemenatry determining regions. For example, the Kabat numbering system (See Kabat, E.A., et al., Sequences of Protein of immunological interest, Fifth Edition, US Department of Health and Human Services, US Government Printing Office (1991)), the Chothia numbering system (See Chothia & Lesk J. Mol. Biol. 196:901-917 (1987), Chothia et al. Nature 342:878-883 (1989)) or the IMGT numbering system (See IMGT®, the international ImMunoGeneTics information system®. Available online: http://www.imgt.org/). The IMGT numbering system is routinely used and accepted as a reliable and accurate system in the art to determine amino acid positions in coding sequences, alignment of alleles, and to easily compare sequences in immunoglobulin (IG) and T-cell receptor (TR) from all vertebrate species. The accuracy and the consistency of the IMGT data are based on IMGT-ONTOLOGY, the first, and so far unique, ontology for immunogenetics and immunoinformatics (See Lefranc. M.P. et al., Biomolecules, 2014 Dec; 4(4), 1102-1139). IMGT tools and databases run against IMGT reference directories built from a large repository of sequences. In the IMGT system the IG V-DOMAIN and IG C-DOMAIN are delimited taking into account the exon delimitation, whenever appropriate. Therefore, the availability of more sequences to the IMGT database, the IMGT exon numbering system can be and “is used” by those skilled in the art reliably to determine amino acid positions in coding sequences and for alignment of alleles. Additionally, correspondences between the IMGT unique numbering with other numberings (i.e., Kabat) are available in the IMGT Scientific chart (See Lefranc. M.P. et al., Biomolecules, 2014 Dec.; 4(4), 1102-1139).

The term “hypervariable region” or “variable region” refers to the amino acid residues of an antibody that are typically responsible for antigen-binding. The hypervariable region generally comprises amino acid residues from a “complementarity determining region” or “CDR” (e.g., around about residues 24-34 (LI), 50-56 (L2) and 89-97 (L3) in the V_{L}, and around about 31-35 (HI), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (LI), 50-56 (L2) and 89-97 (L3) in the V_{L}, and 26-32 (HI), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop” VCDR (e.g., residues 27-38 (LI), 56-65 (L2) and 105-120 (L3) in the V_{L}, and 27-38 (HI), 56-65 (H2) and 105-120 (H3) in the V_{H} when numbered in accordance with the IMGT numbering system; Lefranc, M.P. et al. Nucl. Acids Res. 27:209-212 (1999), Ruiz, M. e al. Nucl. Acids Res. 28:219-221 (2000)). Optionally, the antibody has symmetrical insertions at one or more of the following points 28, 36 (LI), 63, 74-75 (L2) and 123 (L3) in the V_{L}, and 28, 36 (HI), 63, 74-75 (H2) and 123 (H3) in the V_{H} when numbered in accordance with AHo; Honneger, A. and Plunkthun, A. J. Mol. Biol. 309:657-670 (2001)).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is said to specifically bind an antigen when the dissociation constant is ≤ 1 µM; preferably ≤ 100 nM and most preferably ≤ 10 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is said to specifically bind to the Toll-like Receptor 4 (TLR4)/MD-2 complex or to TLR4 when not complexed to MD-2, when the equilibrium binding constant (K_{d}) is <1 µM, preferably ≤ 100 nM, more preferably ≤ 10 nM, and most preferably ≤ 100 pM to about 1 pM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the invention include the nucleic acid molecules encoding the heavy chain immunoglobulin molecules shown herein, and nucleic acid molecules encoding the light chain immunoglobulin molecules shown herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the invention comprise the heavy chain immunoglobulin molecules shown herein, and the light chain immunoglobulin molecules shown herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

The term oligonucleotide referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the invention are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes Oligonucleotides linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology - A Synthesis (2nd Edition, E.S. Golub and D.R. Gren, Eds., Sinauer Associates, Sunderland7 Mass. (1991)). Stereoisomers (e.g., D- amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε -N-acetyllysine, O-phosphoserine, N- acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single- stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide- containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur- containing side chains is cysteine and methionine. Preferred conservative amino acids substitution groups are: valine-leucine-isoleucine, phenylalanine-tyrosine, lysine-arginine, alanine valine, glutamic- aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, preferably at least 14 amino acids long’ more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has specific binding to TLR4/MD2 complex or TLR4 alone, under suitable binding conditions. Typically, polypeptide analogs comprise a conservative amino acid substitution (or addition or deletion) with respect to the naturally- occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p.392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N , ³⁵S, ⁹⁰Y, ⁹⁹Tc, ¹¹¹In _{>} ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

As used herein, the term “viral infection” refers to one or more RNA viruses belonging to families Coronaviridae, Bunyaviridae, Filoviridae, Flaviviridae, Paramyxoviridae, Picornaviridae, Orthomyxoviridae or Rhabdoviridae. Other embodiments include one or more viruses belonging to families Hepadnaviridae, Reoviridae or Retroviridae. Another embodiment includes one or more DNA viruses belonging to families Adenoviridae, Herpesviridae, Papillomaviridae or Papovaviridae.

As used herein, the term “coronavirus infection” or “Coronaviridae infection” refers to an infection caused by a virus belonging to the family Coronaviridae. For example, coronavirus may include but are not limited to 29E alpha coronavirus, NL63 alpha coronavirus, OC43 beta coronavirus, HKU1 beta coronavirus, Middle East Respiratory Syndrome beta coronavirus (MERS-CoV or MERS), Severe Acute Respiratory Syndrome beta corona virus (SARS-CoV or SARS), novel coronavirus that causes coronavirus disease 2019 (COVID-19, 2019-nCoV or SARS-CoV-2), or a mutant and/or variant thereof. Variants of SARS-CoV-2 include but are not limited to B.1.1.7, B.1.351, P.1, B.1.427 or B.1.429. It is to be understood that new variants of coronavirus with novel mutations or sets of mutations can arise.

The term patient includes human and veterinary subjects.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1 - Pharmacology and Toxicology Studies of NI-0101 Anti-Tlr4 Antibody and 5e3 Mouse Surrogate Anti-Tlr4 Antibody

### 1.1 Non-clinical Pharmacology Studies

### 1.1.1. In Vitro Binding Characteristics of NI-0101

NI-0101 is a humanized IgGlk mAb that specifically binds to human TLR4 independently of MD-2, inhibiting signaling by blocking receptor dimerization.

As the target for NI-0101 is membrane-bound and not soluble, a combination of Kinetic Exclusion Assay (KinExA) and ELISA was used to determine the average binding affinity to human TLR4. These experiments demonstrated that NI-0101 has an average dissociation constant (Kd) value of 139 pM for human TLR4.

Immunoglobulin FcRs are expressed on hematopoietic cells and play a role in immune complex-mediated activation or inhibition of cellular function. Binding of human IgG to leukocytes occurs via the human Fc gamma (γ) R family that includes FcγRI (CD64), FcγRIIa (CD32a), FcγRIIb (CD32b), FcγRIIIa (CD16a) and FcγRIIIb (CD16b). Among these receptors, FcγRIIa has a polymorphism at position 131 (arginine (R) or histidine (H)) and FcγRIIIa has a polymorphism at position 158 (valine (V) or phenylalanine (F)), which result in different binding affinities for IgG. Regarding binding affinity, FcyRI binds with the highest affinity to human IgG (10⁻⁸ to 10⁻¹⁰ M) and thus, can engage monomeric IgG, while FcγRII or FcγRIII bind with a lower affinity (10⁻⁵ to 10⁻⁷ M) and require multimeric immune complexes for their interaction. FcgRs also display differing effector functions, for example, only FcγRIII engagement provokes ADCC. Therefore, two mutations (see Section Error! Reference source not found.) were introduced into the Fc portion of NI-0101 to specifically remove the binding to FcγRIII, thus avoiding the potential for ADCC. As summarized in Table 9, the dissociation constants of NI-0101 demonstrate no interaction with FcγRIII while maintaining binding to FcγRI and FcγRII. Furthermore, the affinity to the neonatal FcR (FcRn), involved in homeostasis of IgG serum levels, was not affected by the mutations. As a benchmark, the original non-mutated version of NI-0101, Hu 15C1, was included for comparison.

TABLE 9

Binding Affinity of NI-0101 and its Parental Antibody, Hu 15C1, to Human TLR4 and Human FcRs, as Determined by KinExA (hTLR4) and Surface Plasmon Resonance (FcγRI, FcγRIIa, FcγRIIb, FcγRIII and FcRn), and Expressed in Molar

| | | | | | |
|---|---|---|---|---|---|
| Analyte | hTLR4 | FcγRI | FcγRIIa 131R | FcγRIIa 131H | FcγRIIb |
| Hu 15C1 | 1.39E⁻¹⁰ | 3.40E⁻⁸ ± 0.82 | 2.07E⁻⁶ ± 1.15 | 1.58E⁻⁶ ± 0.56 | 1.15E⁻⁵ ± 0.21 |
| NI-0101 | | 4.73E⁻⁸ ± 0.13 | 1.51E⁻7 ± 0.15 | 3.11E⁻⁷ ± 0.37 | 8.70E⁻⁷ ± 0.38 |

### 1.1.2 In Vitro Activity of NI-0101 on Relevant Biological Targets

### 1.1.2.1. NI-0101 Inhibition of Human TLR4 Biological Activity

NI-0101 blocked IL-6 production induced by three different doses of LPS (0.05, 0.25, and 1.25 ng/mL) with similar capacity. The results suggested that the inhibitory capacity of NI-0101 is independent of ligand concentration. Furthermore, NI-0101 inhibits LPS, Tenascin-C and nickel mediated cytokine production in human primary macrophages with similar potency (IC50 = 10.4 pM for LPS, 22 pM for Tenascin-C and 15 pM for nickel). These experiments demonstrated that NI-0101 blocks TLR4 signalling in a ligand-independent manner.

### 1.1.2.2. Engagement of FcγRs by the Fc Portion of NI-0101

The consequences of human FcγRI and FcγRIIa engagement by NI-0101 on a monocytic cell line were investigated. When NI-0101 was able to engage TLR4 and FcγRI or FcγRIIa, it became more potent at blocking TLR4-mediated cytokine release. These results demonstrated that FcγRI and FcγRII play a role in the inhibitory mechanism of NI-0101 on inflammatory cells. However, NI-0101 engagement did not induce signalling through the FcγRs.

### 1.1.2.3. Human FcgRIIa Polymorphism and NI-0101

As mentioned previously, FcyRIIa has a polymorphism at amino acid position 131. The Fc-portion of NI-0101 binds with a two-fold higher affinity to the 131R than to the 131H variant (Table 9). Using whole blood taken from 121 healthy subjects, the distribution of the genotypes was found to be 21% 131RR, 33% 131HH and 46% 131RH. NI-0101 blocked LPS-induced IL-6 release for all three populations. The IC50 of NI-0101 was 128 pM for the 131RR population, 223 pM for the 131RH population and 2191 pM for the 131HH population. Due to the saturation of FcgRI in whole blood by circulating IgGs, in this compartment, the inhibition of IL-6 secretion by NI-0101 was affected by the FcgRIIa 131 genotype of the donors.

### 1.1.2.4. Effect of FcgRI Versus FcgRIIa

The potency of NI-0101 was tested with two myeloid cell lines from the monocytic lineage, THP1 and U937, expressing both FcγRI and FcγRIIa. THP1 carries the FcγRIIa 131HH genotype and U937 the 131RR genotype. NI-0101 potently blocked LPS-induced IL-6 release in both cell lines with a similar IC50; 2.7 pM for THP1 vs 2.0 pM for U937. The results demonstrated that the FcγRIIa polymorphism has minimal effects on NI-0101 potency when both FcγRI and FcγRIIa are available for NI-0101 engagement.

### 1.1.2.5. Specificity of NI-0101 for Human TLR4

The ability of NI-0101 to block the activation of different TLRs was tested. NI-0101 blocked TLR4 activation, but not other TLRs.

### 1.1.2.6. Epitope and Cross Reactivity of NI-0101

The epitope of NI-0101 on human TLR4 was found to span amino acid residues 289 to 375, an area involved in TLR4 dimerization. Within this hypervariable region, three boxes (amino acid residues 328 to 329, 349 to 351 and 369 to 371) were identified as essential for the binding of NI-0101 to human TLR4. In addition, the binding of NI-0101 to TLR4 is independent of MD-2. The amino acid differences in the NI-0101 epitope of human TLR4 and the orthologs of other species are predicted to prevent NI-0101 from cross-reacting with TLR4 from any standard laboratory species. This prediction was confirmed in mice, rabbits and cynomolgus monkeys.

In conclusion, both structural information and laboratory results concur that NI-0101 does not cross-react with TLR4 from any standard laboratory species.

### 1.1.2.7. TLR4 Receptor Density

Receptor density analysis in blood from healthy donors demonstrated that monocytes expressed the highest number of TLR4 molecules per cell (1888 ± 639 per cell). The granulocytes were shown to have the second highest expression level (1307 ± 490 per cell). The total number of TLR4 molecules was calculated to be 5.38 ± 1.31 × 109 TLR4 molecules/mL of blood (around 9 pM), with granulocytes and monocytes together representing approximately 84% of the source for TLR4 molecules in the blood.

### 1.1.3. In Vitro Characterization of the Mouse Surrogate mAb 5E3

As NI-0101 does not cross-react with TLR4 from any standard laboratory animal species, a surrogate antibody was developed to be used during preclinical development of NI-0101 according to the relevant guidelines (ICH S6 (R1) CHMP/ICH/731268/1998 and CHMP/SWP/28367/07). An anti-mouse TLR4 mAb was generated by immunizing a rat and, from a resulting hybridoma, the variable region was sequenced and grafted onto a mouse IgG2a κ Fc. This chimeric surrogate mAb is referred to as 5E3. As summarized in Table 10, 5E3 displays characteristics for mouse TLR4 similar to that of N1-0101 for human TLR4 and thus is deemed a suitable surrogate reagent to conduct studies that are representative of NI-0101. The following sections provide the supporting evidence in more detail.

TABLE 10

Comparison of 5E3 with NI-0101

| | | |
|---|---|---|
| | NI-0101 | 5E3 |
| TLR4 Affinity | 139 pM | 27 pM |
| MD-2 independent binding | ü | ü |
| Epitope on TLR4 | In region critical for receptor dimerization | In region critical for receptor dimerization |
| Blockade of LPS induced TLR4 signalling | IC50 2.19 nM (324 ng/mL)^{a)} | IC50 1.16 nM (173 ng/mL) ^{b)} |
| FcγR binding involvement | ü | ü |

a) 95% confidence interval: 1.664 - 2.767 nM in human whole blood (for FcγRIIa_{HH}).
b) 95% confidence interval: 0.582 - 2.361 nM in mouse whole blood.

### 1.1.3.1. Binding Affinity of 5E3 to Mouse TLR4 and Epitope Characterization

5E3 has an average affinity constant (Kd) value of 27 pM for mouse TLR4. The value is in the same picomolar range as that of NI-0101 for human TLR4.

The epitope of 5E3 was shown to be in a region required for TLR4 dimerization, mimicking NI-0101 binding to human TLR4. Furthermore, 5E3 binds to mouse TLR4 independently of mouse MD-2, similar to NI-0101 for human TLR4.

### 1.1.3.2. Binding Affinity of 5E3 to Mouse FcyRs

5E3 has an affinity for mouse FcyRI and FcyRII similar to that of NI-0101 for comparable human receptors.

TABLE 11

Binding affinity of 5E3 to mouse Fc receptors (Kd in M)

| | | | | | |
|---|---|---|---|---|---|
| Analyte | mFcγRI | mFcγRII | mFcγRIII | mFcγRIV | mFcRn |
| 5E3 | 5.08E⁻⁰⁸ ± 0.63 | 3.26E⁻⁰⁶ ± 1.61 | 5.05E⁻⁰⁷ ± 0.94 | 1.13E⁻⁰⁷ ± 0.25 | 2.93E⁻⁰⁹ ± 0.02 |

### 1.1.3.3. Blockade of LPS-induced IL-6 Production by 5E3 and the Contribution of FcγR Engagement to the Inhibitory Potency of 5E3

5E3 blocked LPS-induced cytokine production in mouse whole blood with an average IC50 of 1.16 nM. This value is within an acceptable range as compared to that of NI-0101 in human whole blood from FcyRIIa 131HH donors (IC50 of 2.19 nM). In addition the Fc portion of 5E3 is involved in its mechanism of action.

Overall, the in vitro characterization of 5E3 demonstrated the equivalence of function of this surrogate antibody versus the clinical candidate, NI-0101. This provides adequate justification for the use of this surrogate for both preclinical proof-of-principle and efficacy studies as well as for the purpose of safety assessment.

Furthermore, the surrogate antibody, 5E3, was produced using a similar process as that for NI-0101 with the exception that purification included only the first two chromatography steps and a low pH hold. The results of analytical testing demonstrated the quality of the material to be suitable for pharmacology, pharmacokinetics and toxicology studies. The batch analysis of 5E3 toxicology material and NI-0101 GMP drug substance showed comparative results.

### 1.1.4. Pharmacology Studies Supporting the Role of TLR4 in the Pathogenesis of ARDS

### 1.1.4.1. LPS-Induced Endotoxemia Model in CD1 Mice

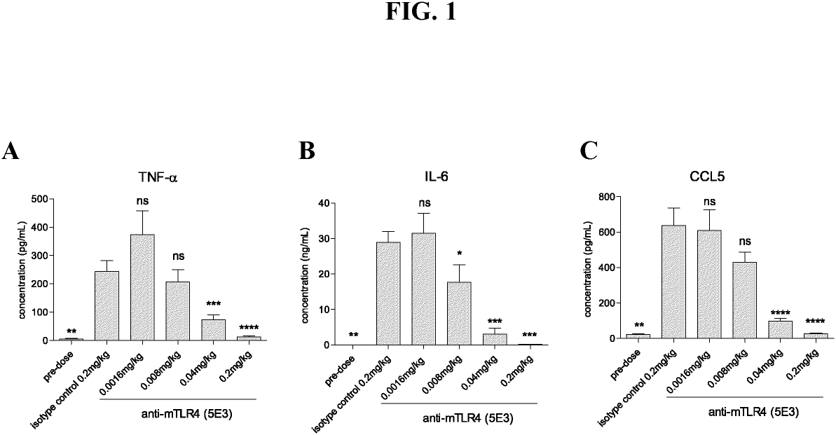

5E3 was used in a systemic murine model of inflammation (endotoxemia). Pretreatment i.v. with 5E3 prior to the i.v. injection of 1 µg/kg of LPS (equivalent to a human dose of 4 ng/kg decreased cytokine/chemokine serum levels in a dose-dependent manner (FIGS. 1A-1C). In this experiment a 0.2 mg/kg dose (human equivalent dose, 0.017 mg/kg) of 5E3 was the saturating (Emax) dose, 0.04 mg/kg (human equivalent dose, 0.003 mg/kg for the HH genotype) was the minimally effective dose. CD1 mice were injected i.v. with 0.2, 0.04, 0.008, or 0.0016 mg/kg of 5E3 mAb or 0.2 mg/kg of isotype control mAb. 1 h later the mice were injected i.v. with 1 µg/kg of LPS. 2 h later the mice were euthanized and serum cytokines and chemokines were measured using the Luminex technology. Pre-dose represents cytokine and chemokine levels 24 h before mAb treatment. FIGS. 1A-1C shows that 5E3 inhibits LPS-induced cytokine storm in mouse blood in a dose dependent manner.

### 1.1.4.2. LPS Instillation Model of Acute Lung Injury (ALI)

TLR4 has been reported to play an important role in models of ALI. To determine whether TLR4-blockade had the potential to ameliorate ALI, 5E3 was evaluated in a mouse LPS-lung-instillation model.

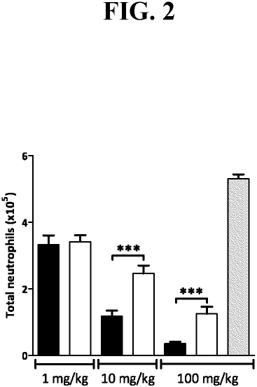

LPS induced a large neutrophil influx and cytokine release in the lung. Pretreatment i.v. with 5E3 blocked LPS-induced neutrophil recruitment and cytokine/chemokine accumulation in the lung in a dose-dependent manner (Error! Reference source not found.). The accumulation of TNFα and IL-6 (induced via the MyD88 pathway as well as CCL5 (upregulated via the TRIF pathway) was blocked by 5E3 treatment (FIG. 2 shows the inhibitory effect of 5E3 and 5E3 IgG2a D265A mutant on LPS-dependent neutrophil recruitment into the lung.

A-3C). These results suggest that 5E3 neutralized both the MyD88 and TRIF-dependent TLR4 pathways in vivo. The saturating (Emax) dose of 5E3 in this model was 33 mg/kg (human equivalent dose, 2.7 mg/kg). The minimum effective dose, which resulted in significant reduction of cytokine production and neutrophils influx to the lung, was 0.33 mg/kg (human equivalent dose, 0.027 mg/kg for the FcγRIIa 131HH genotype).

The contribution of the Fc portion of 5E3 to its inhibitory potency was also assessed in this model. The D265A mutant of 5E3, which fails to engage FcyRs, was approximately 10-fold less potent at inhibiting LPS-induced neutrophil recruitment and cytokine release compared with the wild type (WT) antibody (Error! Reference source not found. and FIG. 2 shows the inhibitory effect of 5E3 and 5E3 IgG2a D265A mutant on LPS-dependent neutrophil recruitment into the lung.

A-3C), with 100 mg/kg of 5E3 D265A reducing neutrophil numbers and cytokine concentrations to levels similar to those measured in the 10 mg/kg 5E3 group. These results demonstrated that the Fc portion also enhances the inhibitory potency of 5E3 in vivo in the LPS lung instillation model of ALI. Mice received an i.v. injection of indicated concentrations of 5E3 (black bars), 5E3 IgG2a D265A (blank bars) or isotype control (grey bar) (n = 10 for each group), followed by the i.n. administration of 10 µg LPS into the lung; 24 hrs later, bronchoalveolar lavage (BAL) fluid was collected and used to count the absolute number of neutrophils. FIG. 2 shows the inhibitory effect of 5E3 and 5E3 IgG2a D265A mutant on LPS-dependent neutrophil recruitment into the lung.

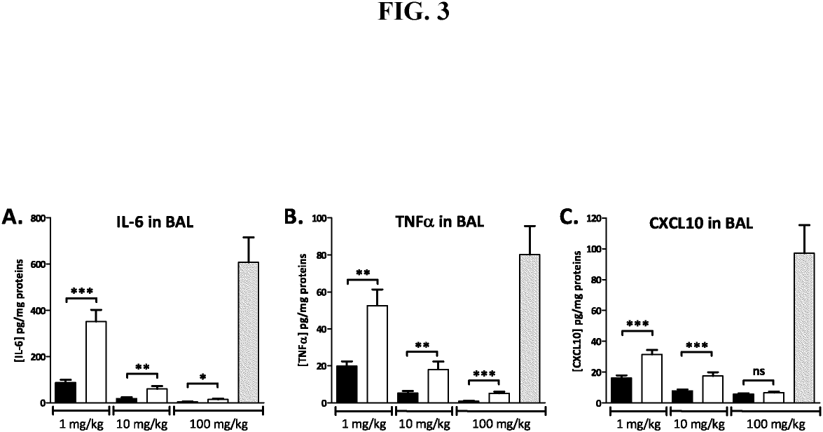

Mice were treated as described in the legend for Error! Reference source not found. 5E3 (black bars), 5E3 IgG2a D265A (blank bars) or isotype control (grey bar). BAL supernatants were separated from cells by centrifugation. Cytokines and chemokines were quantified. FIGS. 3A-3C shows that 5E3 inhibits LPS-induced IL-6, TNFα and CXCL10 secretion into the BAL fluid.

This study confirms TLR4 as a potential therapeutic target in ALI. The ability of 5E3 to co-engage TLR4 and the FcyR in vivo improves its potency to inhibit an LPS-induced pathology. Non-clinical Pharmacokinetics of 5E3

The pharmacokinetic profile of 5E3 was evaluated in one single dose PK/PD study and after multiple injections, as part of the 4-week and 16/26-week GLP toxicology studies.

### 1.1.5. Single Dose PK/PD Profile of the Surrogate Antibody, 5E3

A single i.v. dose of 50 or 75 mg/kg of 5E3 was administered to CD1 mice. 5E3 showed a biphasic, dose-independent and linear pharmacokinetic (PK) profile. The terminal half-life (T½) was approximately 15.4 days. LPS-induced IL-6-production ex vivo was inhibited for up to 14 days post-injection.

### 1.1.6. 4-Week Repeated-Dose Pharmacokinetics of 5E3

As part of the 4-week GLP toxicology study, a satellite study of 30 mice/sex/group was assigned for toxicokinetic evaluation. Mice were i.v. administered 5E3 at 60-, 120- or 240 mg/kg/week for 4 weeks.

The overall analysis indicated the following:

Cmax = 7315 µg/mL after the last injection of 240 mg/kg (average of male and female values).

AUC0-168h = 518000 µg.hrs/mL at 240 mg/kg at day 28 (average of male and female values).

Exposure tended to increase proportionally during week 1 and then in a sub-proportional manner during week 4 across the 60 to 240 mg/kg dose range.

t_{½} = 16d (average male and female values).

Accumulation was observed upon once-weekly repeated administration.

The volume of distribution Vz estimates were greater than the plasma volume in the mouse suggesting there is tissue distribution.

At the high doses used in toxicological studies, the kinetics of 5E3 were linear.

There was no evidence of immunogenicity.

### 1.1.7. 16/26-Week Repeated-Dose Pharmacokinetics of 5E3

As part of the combined 16/26-week GLP toxicity study of 5E3 in mice, a satellite study of 12-21 mice/sex/group was assigned for toxicokinetic evaluation. Mice were administered with 5E3 i.v. once a week at 40-, 80- and 160 mg/kg/week for a total of either 16 or 26 weeks.

Mean trough concentrations achieved maximum values between Weeks 8 and 16 at all dose levels. Accumulation ratios for mean trough concentrations ranged from 0.687 to 2.05 indicating some accumulation of 5E3 from Week 4 where concentrations essentially achieved steady state.

Mean C15 concentrations achieved maximum values between Weeks 8 and 16 at all dose levels. Accumulation ratios for mean C15 concentrations ranged from 1.03 to 2.47 indicating some accumulation of 5E3 from Week 4, where concentrations, essentially, achieved steady state conditions.

Increases in mean trough and C15 concentrations were, overall, proportional with respect to dose level between 40 and 80 mg/kg/week but were sub-proportional at 160 mg/kg/week.

Following the last dose administration at 160 mg/kg/week, the half-lives of 5E3 were estimated to be about 20 days and 13 days in males and females, respectively, in agreement with the almost complete elimination of 5E3 observed within 13 weeks following the last dose administration.

There were no notable gender related differences in the serum concentrations of 5E3.

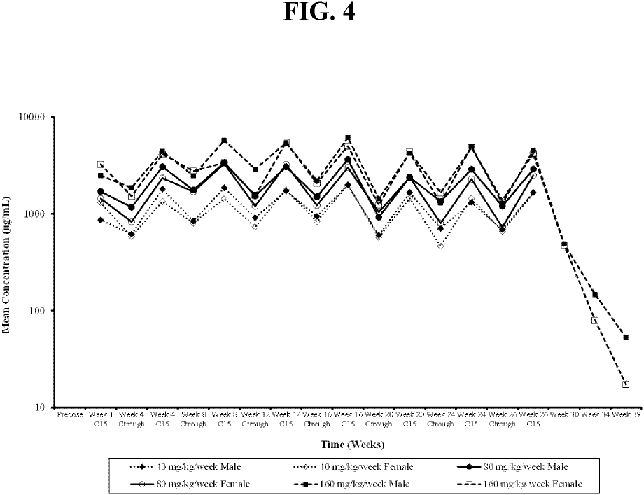

The AUC(tau), following the last dose, was 20300 and 19500 µg.day/mL in males and females, respectively. Exposure profiles until weeks 26 (main phase animals) or 39 (recovery animals) of the study are presented in Error! Reference source not found.. Immunogenicity was not assessed during this 16/26-week study but there was no evidence for the development of anti-drug antibodies (ADAs) from the PK/drug concentration profiles. There were no study findings related to ADAs and exposure to 5E3 was maintained throughout the dosing phase of the study. For these reasons, the lack of ADA data does not impact on the interpretation of the study outcome. FIG. 4 shows mean serum concentrations of 5E3 in mice following intravenous administration of 5E3.

### 1.2. Toxicology

As NI-0101 does not cross-react with any standard laboratory species, the safety assessment of the compound included toxicology studies performed with the murine surrogate antibody, 5E3, and in vitro experiments conducted with NI-0101.

### 1.2.1. Single-Dose Toxicology Studies

A single dose study to assess effects on general activity, behavior and body temperature in the mouse, following a single bolus i.v. administration of 5E3 (at 60, 120 and 240 mg/kg), is described under safety pharmacology. No behavioral, physiological or body temperature changes were observed in the 5E3 treated animals when compared with the vehicle-treated group over an 8-day period.

### 1.2.2. Repeated-Dose Toxicology Studies

### 1.2.2.1. 4-Week Repeated-Dose Toxicology Study in Mice With the Surrogate Antibody, 5E3

The objective of this GLP-compliant study (Covance study number 8248643) was to investigate the toxicity of blocking TLR4 using 5E3 once-weekly via an i.v. (bolus) administration to mice for 4 weeks. An assessment of the reversibility of toxicity was made during an 8-week treatment-free period.

Four groups of CD-1 mice were administered with bolus i.v. 5E3 dosages of 0 (vehicle control), 60, 120 or 240 mg/kg/week (Days 1, 8, 15 and 22) for four weeks.

Nine mice/sex/group were bled at weeks 2 and 3 for hematology and biochemical analysis, respectively. Twelve mice/sex/group were euthanized one week after the final dose (Day 29), with the remaining 6 mice/sex/group (control and high dose groups only) euthanized after an 8 week recovery period. An additional 30 satellite mice/sex/group were administered the same doses of 5E3 antibody, according to the same schedule and used for a toxicokinetic evaluation.

No effects on body weight, food consumption, weight of the main organs (i.e., brain, heart, kidney, testes/epididymis, spleen and liver) were observed when 5E3 was administered by i.v. (bolus) injection to mice once weekly for 28 days at dose levels up to 240 mg/kg/week. Clinical and histopathology examinations did not reveal any abnormalities.

No 5E3-related changes were observed in any of the parameters recorded during the study at any dose level. Therefore, the No-Observable-Adverse-Effect-Level (NOAEL) was assumed to be the highest dose tested, i.e., 240 mg/kg/week.

### 1.2.2.2. Combined 16/26-week Repeated-Dose Toxicology Study in Mice With the Surrogate Antibody, 5E3

The objective of this GLP-compliant study (Covance study number 8296471) was to determine the toxicity of 5E3 following weekly i.v. administration to Cr1:CD1(ICR) mice for up to 26 weeks, with an assessment of the reversibility of any toxicity during a 13-week recovery period.

The study was a combination design that involved the treatment of mice for either 16 or 26 weeks, up to 160 mg/kg/week of the test article. Toxicokinetic and immunotoxicity assessments were also performed.

Toxicokinetic data are discussed in Section [00367].

There were no mortalities during the study related to 5E3.

There were no 5E3-related changes on bodyweights, food consumption, haematology, clinical chemistry, ophthalmoscopy or immunotoxicity (T-cell dependent antibody response to Keyhole Limpet Hemocyanin (KLH)) parameters after i.v. administration (bolus) to the mice once weekly for 26 weeks at dose levels up to and including 160 mg/kg/week.

There was no organ weight and/or organ weight ratio changes, or macroscopic or microscopic findings considered to be related to 5E3.

No 5E3-related changes were observed in any of the parameters recorded during the study at any dose level. Therefore, the No-Observable-Adverse-Effect-Level (NOAEL) was assumed to be the highest dose tested, i.e., 160 mg/kg/week.

Taken together, the toxicology studies have not revealed any safety concerns up to and including repeated doses of 240 mg/kg for 4 weeks and chronic repeated dosing up to and including 160 mg/kg for 26 weeks.

### 1.2.3. Genotoxicity

Genotoxicity studies have not been performed and are not considered necessary for the development of a mAb. NI-0101 is produced in mammalian cells and the preparation methods do not include steps that are unusual or include the use of reactive substances. NI-0101 is a mAb of the IgG1 isotype and does not contain any non-natural amino acids, chemical linkers or chelators. NI-0101 acts by binding to TLR4, for which there is no evidence of a mutagenic effect. NI-0101 is not expected to have an action, which is related to DNA integrity. For these reasons, genotoxicity studies will not be performed (in accordance with ICH S6).

### 1.2.4. Carcinogenicity

No carcinogenicity studies have been performed. According to ICH S6(R1), rodent bioassays (or short-term carcinogenicity studies) with homologous products (e.g. 5E3 mAb) are generally of limited value to assess carcinogenic potential of the clinical candidate. Therefore, no rodent carcinogenicity studies have been performed with the 5E3 mAb.

### 1.2.5. Reproductive and Developmental Toxicology

No reproductive toxicology studies have been conducted.

However, histological examination of the reproductive tract was assessed in the 4-week and 16/26-week GLP-compliant mouse toxicity studies with the surrogate antibody, 5E3, and no 5E3-related adverse findings were noted.

No specific developmental toxicity studies have been conducted so far, but such studies are not considered necessary at this stage of development of a mAb.

### Example 2 A Randomized, Double-blind, Placebo-controlled Study to Evaluate the Safety and Efficacy of NI-0101 + Soc Vs. Placebo + Soc in Adult Hospitalized Patients With Covid-19

### 1. Objectives and Endpoints

### 1.1 Objectives

### Main Study

### Primary Objective

To evaluate the clinical efficacy of NI-0101 + SOC vs. Placebo + SOC in adult hospitalized patients with mild to severe COVID-19 disease defined as WHO-COVID Severity scale score of 3 - 6.

### Secondary Objective

To evaluate the safety of NI-0101 in adult hospitalized patients with mild to severe COVID-19 disease defined as WHO-COVID Severity scale score of 3 - 6.

### Sub (Exploratory) Study

### Primary Objective

To evaluate the clinical efficacy of NI-0101 + SOC vs. Placebo + SOC in adult hospitalized patients with very severe COVID-19 disease defined as ventilation requirement for more than 5 days or WHO-COVID Severity scale score of 7.

### Secondary Objective

To evaluate the safety of NI-0101 in adult hospitalized patients with very severe COVID-19 disease defined as ventilation requirement for more than 5 days or WHO-COVID Severity scale score of 7.

### 1.2 Endpoints

The severity of COVID-19 related respiratory disease is assessed on the following WHO nine- point ordinal scale:

| State | Description | Score |
|---|---|---|
| Uninfected | No clinical or virological evidence of infection | 0 |
| Ambulatory | No limitation of Activities | 1 |
| | Limitation of Activities | 2 |
| Hospitalized Mild Disease | No Oxygen Therapy | 3 |
| | Oxygen by mask or Nasal Prongs | 4 |
| Hospitalized Severe Disease | Non-Invasive Ventilation or High-Flow Oxygen | 5 |
| | Intubation and Mechanical Ventilation | 6 |
| | Intubation + Additional Organ Support Pressors, RRT, ECMO | 7 |
| Dead | Death | 8 |

### Primary Efficacy Endpoint

### Main Study

The proportion of patients that are alive and without any need for oxygen support defined as a score of 3 or less in the above scale. The primary endpoint will be assessed at 28-days after treatment initiation.

### Sub (Exploratory) Study

The cumulative mortality rate at 60 days after treatment initiation.

### Secondary Efficacy Endpoints

### Main Study and Sub (Exploratory) Study

Time to therapeutic response (primary efficacy endpoint of the Main Study).

The proportion of patients with clinical improvement, defined as a decrease of two points or more on the WHO 9 – point ordinal scale at Day 28.

The proportion of patients that are alive and discharged home without any need for oxygen support (WHO Scale of ≤ 2) at Day 28.

The proportion of patients that are alive and free of respiratory failure (WHO scale ≤ 4) at Day 28.

The proportion of patients with clinical improvement, defined as a decrease of two points or more on the WHO 9 – point ordinal scale at Day 28.

The proportion of patients with clinical improvement, defined as a decrease of two points or more on the WHO 9 – point ordinal scale at Day 60.

The proportion of patients with clinical improvement, defined as a decrease of one point or more on the WHO 9 – point ordinal scale at Day 60.

The proportion of patients that are alive and discharged home without any need for oxygen support (WHO Scale of ≤ 2) at Day 60.

The proportion of patients that are alive and free of respiratory failure (WHO scale ≤ 4) at Day 60.

Time to clinical improvement by 2 points on the WHO ordinal scale described above.

Time to clinical improvement by 1 point on the WHO ordinal scale described above.

Change in the NEWS-2 Scale at 28 days.

Time to NEWS-2 = 0.

The proportion of patients that experience disease progression, defined as an increase of one point or more in the WHO 9-point ordinal scale, at Day 28.

Ventilator-free days.

Duration of ventilation.

Mortality rate at 28-days and 60-days post-treatment initiation.

Duration of hospitalization.

Time to independence from supplementary oxygen therapy.

Time to normalization of oxygen saturation, defined as room air SpO2/SaO2 > 94% sustained minimum 24 hours.

Change in Sequential Organ Failure Assessment (SOFA) score, while hospitalized.

Radiological response to treatment based on Thoracic Computerized Tomography Scan (CT-Scan) or Chest X-Ray.

Change in cytokines, including IL-6, and C-reactive protein (CRP) levels.

Time to resolution of fever for at least 48 hours without antipyretics.

o Defined as body temperature <37.2° C. (oral), or <37.6° C. (rectal or tympanic) or <36.8° C. (temporal or axillary).

Decision by the attending physician to initiate treatment with another targeted immunomodulator (e.g., dexamethasone).

Change in Berlin ARDS severity scale.

Change in Acute Kidney Injury Network (AKIN) classification.

Change in troponin levels.

### Sub (Exploratory) Study

All the secondary endpoints of the Main Study including the following that will be for the Sub- Study only:

The proportion of patients that are alive without any need for oxygen support defined as a score of 3 or less in the above scale.

Duration on ECMO.

### Safety Endpoints

The number of treatment-emergent adverse events (TEAEs) and serious TEAEs.

### 2. Study Design

### 2.1 Overall Design

This is a multicenter, randomized, double-blind, placebo-controlled, two-stage study to evaluate the safety and efficacy of NI-0101 in adult hospitalized patients with COVID-19. The study will be comprised of:

The Main Study that will be conducted on patients that are hospitalized with mild to severe COVID-19 disease defined as WHO-COVID-19 severity scale score of 3 and 6.

A Sub (Exploratory) Study that will be conducted on patients that have very severe COVID-19 disease defined as ventilation requirement for more than 5 days or WHO- COVID-19 severity scale score of 7.

In addition to receiving standard-of-care (SOC), patients will be randomized (1:1 ratio) to either NI-0101 (NI-0101 + SOC) or Placebo (Placebo + SOC). The study will be performed at approximately 60 investigational sites located in Canada, the United States, LATAM, and Europe.

Patients should provide informed consent before any screening procedures being performed. When the participating subject is not capable of providing informed consent, his or her legal/authorized representative can do so if the applicable laws and regulations of the country/region in which the research is conducted allow. After providing informed consent subjects will undergo screening and those fulfilling all inclusion criteria and none of the exclusion criteria will be accepted into the study.

Following enrollment in the Main Study, eligible subjects will be randomized (1:1) at Baseline to receive an infusion of either NI-0101 or Placebo. All patients will also receive SOC treatment per routine care at each participating site. Randomization will be stratified by site and baseline WHO COVID-19 severity strata defined as Levels 3-4, and Levels 5-6. Patients that are eligible for the Sub (Exploratory) Study will be randomized at a 1:1 ratio to receive an infusion of either NI-0101 or Placebo. For the Sub (Exploratory) study randomization will be stratified by site.

The follow-up duration of each patient will be until 60-days from treatment with the investigational product. All assessments will take place in-hospital except for the 28-day and 60-day assessment which will be by telephone if the patient has been discharged before this assessment.

For Stage 1 (Phase II) of the Main Study, approximately 316 evaluable patients are planned for enrollment that will be randomized equally to the two treatment groups, 158 treated with NI-0101 + SOC, and 158 treated with Placebo + SOC. Allowing for 20% attrition, a total of 396 patients will be enrolled in the Main Study.

One interim analysis will be conducted in each stage which will be reviewed by an Independent Data Monitoring Committee (IDMC). The Interim Analyses will be conducted when data are available for the primary endpoint assessment from 50% of the patients. The results of the Interim Analysis will be used to determine whether the study should be continued or terminated early for futility, or safety concerns. The IDMC review meetings will occur at least every 8 weeks by either a telephone call or a report submitted by the committee chair.

In the Sub-Study, enrolment will be completed when a total of 30 events (deaths) have been observed. Two interim analysis will be performed for the Sub (Exploratory) Study. These will take place after 10 events (deaths) and 20 events, respectively. Based on the currently available estimates it is anticipated that approximately 100 patients will be enrolled in the Sub (Exploratory) Study.

### 2.2 Scientific Rationale for Study Design

Randomization will be used to allocate patients to treatment groups thus reducing bias. Since SOC for the management of COVID-19 differs across investigational sites randomization will be stratified by clinical site. Sites will be encouraged to follow the Regional or NIH Coronavirus Disease 2019 (COVID-19) Treatment Guidelines (https:/www.covid19treatmentguidelines.nih.gov/) as a minimum SOC. Also, due to possible differences in the trajectories of COVID-19 cases depending on the initial severity of the disease, randomization in the Main Study will be further stratified (nested within clinical site) on the basis of COVID-19 severity at the time of enrollment. This will define two strata, specifically (i) mild disease (Level 3-4 of the nine-point COVID-19 severity scale); (ii) severe disease (Level 5-6 of the nine-point COVID-19 severity scale). Therefore, within each site, randomization will be stratified in two strata according to baseline disease severity (level 3 – 4 vs. level 5 – 6).

There is an unmet need for the management of patients at the last stage of COVID-19 illness (Level 7) or that have been on mechanical ventilation for more than 5 days for whom the next stage of disease progression is death. Given the specific, targeted immune modulating activity of NI-0101, reversal of the disease process even in patients with severe/extreme respiratory failure, is a scientifically sound and reasonable expectation. Based on this, the effect of NI-0101 in preventing disease progression (i.e., mortality) in this patient population will be assessed in a Sub (Exploratory) Study.

Given that the selected efficacy outcome measures are objective, patient-centric ascertainment bias is not expected to impact the study results. However, investigator-centric selection or indication bias could be a potential risk. The use of a double-blind design with a placebo control will reduce the possibility of bias.

The proposed adaptive design with sequential group analyses employed is considered appropriate for assessing the therapeutic potential of NI-0101 in the shortest time possible in order to discontinue the study either due to futility or safety concerns. The adaptive design allows the potential modification of the study inclusion criteria to ensure the selection of an enriched patient population that would optimize the detection of a therapeutic benefit from NI-0101. In addition, modifications to the sample size requirements, outcome measures and better understanding of how the evolving standard of care can be taken into account in the overall data analysis and design of the study will be possible with the Interim Analyses.

### 2.3 End of Study Definition

The end of study is defined as the shortest between time of discharge from the hospital or Day 28 assessment as shown in the schedule of events Table 1. A follow-up assessment on Day 28 (in the case of Early Hospital Discharge) and a follow-up assessment on Day 60 will be conducted for all randomized patients, regardless of treatment adherence or hospitalization status, for adequate evaluation of safety.

### 3. Study Population

### 3.1 Inclusion Criteria Main Study:

Subjects will be eligible for participation in the study if they meet all the following inclusion criteria:

Men and women ≥18 years of age at the time of consent.

Laboratory-confirmed diagnosis of COVID-19.

Hospitalized for COVID-19 related disease.

Patient belongs to one of the following four categories in the nine-point WHO COVID-19 severity scale:

Hospitalized, not requiring supplemental oxygen – Level 3 of the nine-point COVID-19 severity scale.

Hospitalized, requiring oxygen therapy by mask or nasal prongs – Level 4 of the nine-point COVID-19 severity scale.

Hospitalized, requiring non-invasive ventilation or high flow oxygen – Level 5 of the nine-point COVID-19 severity scale.

Hospitalized, requiring intubation and mechanical ventilation – Level 6 of the nine- point COVID-19 severity scale.

For women of childbearing potential involved in any sexual intercourse that could lead to pregnancy: Negative pregnancy test and willingness to use contraceptive (consistent with local regulations) during 11 weeks after the study drug injection (five half-lives).

Note: The use of contraceptive methods does not apply to subjects who are abstinent for at least 4 weeks before Day 1 and will continue to be abstinent from penile-vaginal intercourse 11 weeks after study drug injection (five half-lives). The reliability of sexual abstinence needs to be evaluated in relation to the duration of the clinical trial and the preferred and usual lifestyle of the participant.

Note: A woman of non-childbearing potential is defined as follows:

Age ≥60 years of age.

Has had surgical sterilization (hysterectomy, bilateral oophorectomy, or bilateral salpingectomy)

Has had a cessation of menses for at least 12 months without an alternative medical cause, and a follicle-stimulating hormone (FSH) test confirming nonchildbearing potential (refer to laboratory reference ranges for confirmatory levels).

Informed consent obtained from any patient capable of giving consent, or, when the patient is not capable of giving consent, from his or her legal/authorized representatives.

### Sub (Exploratory) Study

All the inclusion criteria of the Main Study with the following substitution for inclusion criterion (4):

Hospitalized, requiring ventilation for more than 5 days or intubation + additional organ support – pressors, RRT, ECMO – Level 7 of the nine-point COVID-19 severity scale.

### 3.2 Exclusion Criteria

Subjects will not be eligible for participation in the study if they meet any of the following criteria at Baseline:

The Subject is a female who is breastfeeding or pregnant.

Known hypersensitivity to NI-0101 or its excipients.

In the opinion of the investigator, progression to death is imminent and inevitable within the next 48 - 72 hours, irrespective of the provision of treatment.

Active participation in other immunomodulator or immunosuppressant drug clinical trials.

Participation in COVID-19 antiviral, anticoagulant and convalescent plasma trials may be permitted; however, the decision to enroll a patient who is participating in other clinical trials will be dealt with on a case-by-case basis.

Treatment with immunomodulator or immunosuppressant drugs, including but not limited to TNF inhibitors, and anti-IL-1 agents within 5 half-lives or 30 days (whichever is longer) before randomization. (Note treatment with immunomodulator or immunosuppressant drugs, such as corticosteroids, as part of SOC is permitted).

Known other clinical condition that contraindicate NI-0101 and cannot be treated or solved according to the judgment of the clinician.

### 3.3 Discontinuation and Loss to Follow-Up

Subjects have the right to withdraw from the study at any time for any reason without penalty.

Should a subject be withdrawn from the study, a complete final evaluation for Early Termination visit, should be made with an explanation of why the subject is withdrawing from the study.

Subjects who discontinue from the study will not be replaced. Possible reasons for discontinuation include the following:

The subject may withdraw from the study for any other reason, including withdrawal of consent.

The sponsor or regulatory authorities, for any reason, stop the study. In this case, all subjects will be discontinued from the study. The investigator will immediately, on discontinuance of the study by the sponsor, in its entirety or at a clinical trial site, inform both the subjects and the research ethics board of the discontinuance, provide them with the reasons for the discontinuance and advise them in writing of any potential risks to the health of subjects or other persons.

### Loss to Follow-Up

Given the in-hospital nature of the study loss to follow-up is not anticipated in the study.

### 3.4 Screen Failures

Screen failures are defined as individuals who consent to participate in the clinical trial but are not subsequently randomly assigned to the study products. A minimal set of data on screen failure patients is required to assess the possibility of selection bias and to respond to queries from regulatory authorities. Minimal information includes demographics, disease severity, and reason for screen failure.

Individuals who do not meet the criteria for participation in this trial (screen failure) may be rescreened once if deemed acceptable by the investigator. Rescreened subjects should be assigned a different subject Baseline visit, including signature of a new consent form, will then be performed.

### 4. Treatment

### 4.1 Study Treatment Administered

On Day 1, subjects will be administered a single NI-0101 IV infusion of 15 mg/kg up to a maximum of 1440 mg (10 vials) or a single placebo IV infusion, along with standard-of-care (SOC) COVID-19 treatment. SOC treatment may differ by clinical site however sites will be encouraged to follow the NIH Coronavirus Disease 2019 (COVID-19) Treatment Guidelines as the minimum standard of care. The total follow-up duration of each patient will be until 60-days from treatment with the investigational product.

NI-0101 study treatment and placebo will be provided by Edesa Biotech Research Inc. Treatments related to SOC will not be provided or reimbursed by Edesa Biotech Research Inc.

NI-0101 must only be given by the investigator or delegate as an investigational medicinal product (IMP). Subjects receiving the IMP should be in an environment where appropriate supervision and monitoring are available to allow early detection of adverse reactions and their prompt treatment.

NI-0101 must be diluted and administered by IV infusion over 3 hours. Before administration, the vials containing NI-0101 should be inspected for particulate matter and discoloration. Start and stop times and the total dose in mg administered will be recorded on the eCRF.

More details on the treatment administration will be described in the study manual. Summary details regarding the study products can be found in Table 12 below.

TABLE 12

Study Treatments
Study Treatments

| | | |
|---|---|---|
| Product name | NI-0101 | Placebo |
| Dosage form | 150 mg/mL vial | 0 mg/mL vial |
| Route of administration | IV infusion over a period of 3 hours | IV Infusion over a period of 3 hours |
| Dosing instructions | Single-dose of 15 mg/kg | Single-dose |
| Physical description | Clear of slightly opalescent and colorless to slightly yellow solution | Similar to NI-0101 |
| Source of procurement | Edesa Biotech Research Inc. | Edesa Biotech Research Inc. |

The contents of the label will be in accordance with all applicable regulatory requirements.

### 4.2 Preparation/Handling/Storage/Accountability

### Preparation/Storage/Handling

The drug product is a concentrate for solution for infusion. Each 2 mL vial contains 1.2 mL of a 150 mg/mL solution in formulation buffer (25 mM histidine / 200 mM Arginine, 0.02% (w/v) Polysorbate 80, pH 6.0).

No preservative or bacteriostatic is present in this product; therefore, the vial is for single use only. Standard sterile handling procedures must be used when preparing NI-0101 or matching placebo for administration.

Full instructions for the preparation, including dilution steps, and method for administration of the IMP are available in the IMP manual’s directions for the Preparation and Administration of Individual Doses of Study Drug NI-0101/placebo.

The vials of NI-0101 should be transported and stored under refrigerated conditions of 5 ± 3° C. (41 ± 5° F.) and should not be shaken or frozen.

All study products must be stored in a secure area with access limited to the investigator and authorized site staff. The study product(s) may only be supplied by authorized site staff and may only be administered to subjects enrolled into the study.

The expiry date will be printed on the label.

### Accountability

The investigator or delegate is responsible for maintaining accurate records of the study product received initially and of the study product used. After verification of the study product accountability by the sponsor or designee, the used product will be stored safely until destruction/return. Any discrepancies between amounts dispensed and returned will be explained.

All study product accountability forms and treatment logs must be retained in the investigator’s study files. Product inventory and accountability records will be maintained as per GCP and ICH guidelines. These records must be available for inspection at any time by the sponsor, its designees, or by regulatory agencies.

Further guidance and information for the final disposition of study treatment are provided in the study manual.

### 4.3 Randomization

At the investigational site, each screened subject will be assigned a subject identifier number during screening that will be used on all subject documentation. The subject identifier number will contain the site number and the subject number and will be assigned in numerical order at the Baseline visit based on the chronological order of screening dates (e.g., 02-010 for the 10th subject screened at Site #02).

The study will consist of two treatment groups which will be randomly assigned in a 1:1 ratio: (1) NI-0101 + SOC and (2) Placebo + SOC.

Randomization will occur before first dosing, at Baseline, and will be stratified:

### Main Study

by clinical site; by COVID-19 disease severity at the time of enrollment defined as:
a. Mild to Moderate Disease: Level 3 or 4 of the nine-point COVID-19 severity scale
b. Severe to Critical Disease: Level 5 or 6 of the nine-point COVID-19 severity scale

Based on the above, at each study site randomization will be stratified in two strata as follows:

| COVID-19 Severity (Baseline) | Stratum |
|---|---|
| Level 3 - 4 | 1 |
| Level 5 - 6 | 2 |

Randomization will be blocked to ensure equal 1:1 allocation of treatment groups within each stratum and to maximize matching with respect to SOC received between the two treatment groups.

### Sub (Exploratory) Study

by clinical site:

The randomization list will be generated using validated software and will be kept secured until the treatment allocation blind is broken at the end of the study. This list will be uploaded into an Interactive Web Response/Electronic Data Capture (IWR/EDC) system. The investigator or designee will be able to acquire a randomization number for eligible subjects by utilizing the IWR/EDC system.

Further guidance and information on the randomization procedures will be described in the study manual.

### Blinding

This study will be double-blinded. At all times, treatment and randomization information will be kept confidential and will not be released to the investigator, the study staff, the contract research organization (CRO), or the sponsor’s study team until after the conclusion of the study. With an IWR system, the kits will be blinded already when shipped from the Drug Depot. A minimum amount from each treatment group will be onsite so that it can be allocated to subjects according to the assigned randomization number.

Blinding codes should only be broken in emergencies for reasons of subject safety. Whenever possible, the investigator should contact the sponsor or its designee before breaking the blind. If the blind is broken, the investigator should promptly inform the medical monitor. Documentation of breaking the blind should be recorded with the date/time, the reason(s) why the blind was broken, and the names of the personnel involved.

The subject for whom the blind has been broken will be discontinued from the study and undergo the End of Treatment procedures. In cases where there are ethical reasons to have the subject remain in the study, the investigator must obtain specific approval from the sponsor or its designee for the subject to continue in the study. The reason(s) for discontinuation (the event or condition which led to the unblinding) will be recorded.

During the study, interim analyses will be performed in a blinded fashion by an independent third party (JSS Medical Research). More specifically the analyst will not be aware of the identity of the treatment as the groups will be identified as Group A or Group B. During the conduct of the study, the Sponsor/Study Team will not have access to information about individual treatment assignments, or interim analysis results. The results of the Interim Analyses will be submitted directly to the Independent Data Monitoring Committee.

### Study Treatment Compliance

In-hospital administration of the study drug on Day 1 will be documented on the eCRF. In cases where the study drug was not administered the reason for non-administration will be documented.

### 4.4 Concomitant Therapy

All medications taken throughout the study must be recorded.

Medication entries may be captured as generic or trade names. Trade names should be used for combination drugs. Entries should include as much as possible of the following information: the dose, unit, frequency of administration, route of administration, start date, discontinuation date, and reason for administration. If the medication is discontinued or the dosage is changed, these details must be recorded.

### Prohibited Therapies or Procedures

There are no prohibited treatments or procedures during the course of the study. All details of medications and treatments provided to the patient during the study will be recorded in the concomitant medication sections. If at any point during the study the attending physician decides that treatment with another targeted immunomodulator (e.g.., IL-6 inhibitors) is necessary, this will be recorded on the eCRF.

### 5. Study Assessments

### 5.1 Efficacy Assessments

To assure consistency and reduce variability, the same assessor should perform all assessments on a given subject whenever possible.

### Symptom Assessment

The presence (yes vs. no) of the following symptoms will be assessed daily: Fever, defined as body temperature ≥37.2° C. (oral), or ≥37.6° C. (rectal or tympanic) or ≥36.8° C. (temporal or axillary). The current use of antipyretics will also be recorded in this section of the eCRF; these should also be recorded in the Concomitant Medications section of the eCRF.
Cough with sputum production
Cough with bloody sputum/hemoptysis
Sore throat
Runny nose (rhinorrhea)
Ear pain
Wheezing
Chest pain
Muscle aches (myalgia)
Joint pain (arthralgia)
Fatigue/malaise
Shortness of breath (dyspnea)
Lower chest wall indrawing
Headache
Altered consciousness/confusion
Seizures
Abdominal pain
Vomiting/nausea
Diarrhea
Conjunctivitis
Skin rash
Skin ulcers
Lymphadenopathy
Bleeding (hemorrhage) and site of bleeding
Infections other than SARS CoV2

At Baseline, the assessment will be conducted before study drug administration.

### Nine-Point Ordinal WHO COVID-19 Severity Scale

The severity of COVID-19 related respiratory disease will be assessed on the following nine-point ordinal scale daily:

| State | Description | Score |
|---|---|---|
| Uninfected | No clinical or virological evidence of infection | 0 |
| Ambulatory | No limitation of Activities | 1 |
| | Limitation of Activities | 2 |
| Hospitalized Mild Disease | No Oxygen Therapy | 3 |
| | Oxygen by mask or Nasal Prongs | 4 |
| Hospitalized Severe Disease | Non-Invasive Ventilation or High-Flow Oxygen | 5 |
| | Intubation and Mechanical Ventilation | 6 |
| | Intubation + Additional Organ Support –Pressors, RRT, ECMO | 7 |
| Dead | Death | 8 |

At Baseline, the assessment will be conducted before study drug administration.

### Oxygen Saturation

Oxygen saturation (SpO2) is measured with pulse oximetry. If or when arterial oxygen saturation (SaO2) is also obtained, this will be recorded as well.

At baseline the assessment will be conducted before study drug administration. During hospitalization follow up, oxygen saturation will be assessed as per routine care and as deemed necessary by the hospital staff.

### Sequential Organ Failure Assessment (SOFA) Score

The SOFA score is a simple and objective score that measures organ dysfunction in six organ systems: respiratory, neurological, cardiovascular, hepatic, coagulation, and renal. For each system, a score from 0 to 4 is assigned with an increasing score reflecting worsening organ dysfunction.

At Baseline the assessment will be conducted before study drug administration. During hospitalization follow-up, the frequency of assessing the SOFA score will be per routine care. The SOFA score will also be performed at Day 28 or at Early Hospital Discharge. Depending on the routine care at each clinical site both the SOFA and modified SOFA will be acceptable for data collection.

In addition to the individual organ system scores the following aggregate scores will be analyzed:

Daily Maximum SOFA Score: calculated based on the most severe value for each sub- score in the preceding 24 h.

Total SOFA Score: calculated as the sum of all sub-scores.

### National Early Warning Score - 2 (NEWS-2)

This scale is based on the assessment of six measurements that converges into a single aggregate score. Each parameter is assessed on the variance from the normal values and higher scores indicate worse disease. The following parameters are measured: Respiratory Rate
Oxygen Saturation
Systolic Blood Pressure
Pulse
Level of Consciousness or New Confusion
Temperature

Score for each parameter range from 0 to 3 with higher scores indicating higher deviation from normal values. The total score is increased by 2 points if the patient requires oxygen supplementation to maintain the recommended oxygen saturation levels.

At Baseline, the assessment will be conducted before study drug administration. During hospitalization follow up, NEWS-2 Score will be assessed as per routine care and as deemed necessary by the hospital staff. The NEWS-2 score will also be performed at Day 28 or at early hospital discharge.

The following table summarizes the clinical severity thresholds for the NEWS-2 scale:

| | |
|---|---|
| NEWS-2 Score | Severity |
| Aggregate 0 – 4 | Low |
| Score = 3 in any single parameter | Low - Medium |
| Aggregate 5 – 6 | Medium |
| Aggregate > 6 | High |

### ARDS Severity (Berlin Scale)

The ARDS scales are defined as follows:
Mild: PaO2/FiO2: 200 - 300
Moderate: PaO2/FiO2: 100 - 200
Severe: PaO2/FiO2: < 100

The data required to determine the ARDS level will be collected as per routine care. The baseline ARDS scale will be based on the test results at the date of treatment administration, or the closest date before initiation of treatment. During the study, ARDS severity will be calculated using the results of tests conducted as per routine care.

### Lung Imaging

For the baseline assessment lung imaging results obtained before study drug administration will be used. The method of imaging (Thoracic Computerized Tomography Scan [CT-Scan] or Chest X-Ray) used as well as the bilateral percentage (%) of lung fields with pulmonary infiltrate will be recorded. During hospitalization follow-up, chest imaging will be performed per routine care at each clinical site.

### IL-6 Levels

IL-6 levels (serum or plasma per routine care at each clinical site) will be recorded as per routine care at each clinical site.

At Baseline, the assessment will be conducted before study drug administration.

Also, in a subgroup of 100 patients from selected sites, IL-6 levels will be assessed at Baseline, 12 hrs post IP administration, Day 3, Day 14, and Day 28 or early Hospital Discharge as part of the study, even if not part of routine care.

### Routine Inflammatory Cytokines

Routine inflammatory cytokines (serum or plasma per routine care at each clinical site) will be recorded as per routine care at each clinical site.

At baseline, the assessment will be conducted before study drug administration.

Also, in a subgroup of 100 patients from selected sites, standard panel inflammatory cytokines will be assessed at Baseline, 12 hrs post IP administration, Day 3, Day 14, and Day 28 or early Hospital Discharge as part of the study, even if not part of routine care.

### 5.2 Safety Assessments

### Vital Signs & Anthropometric Measurements

At Baseline, before study drug administration, in addition to oxygen saturation (described above), temperature, heart rate, respiratory rate, and systolic/diastolic blood pressure will be recorded with the subject in a seated position, after having sat calmly for a few minutes.

During hospitalization follow-up, vital signs will be assessed per routine care and abnormal findings that the investigator considers to be clinically significant should be reported as AEs.

Weight (kg) and height (cm) will be collected at the visits specified in Table 1. Height will be measured at the Baseline visit only.

### Physical Examination

A complete physical examination will be completed at Baseline before study drug administration when possible. In the case that a complete Physical Examination is not possible to be conducted, the results of the Examination conducted as per hospital protocols will be recorded.

Including the following sites/systems:
General appearance
Dermatological
Head, eyes, ears, nose, throat (HEENT)
Respiratory
Cardiovascular
Abdominal
Neurological
Musculoskeletal
Lymphatic

During hospitalization follow-up, directed physical examination will be conducted in a symptom- or disease-driven manner and clinically significant abnormal findings should be reported as AEs if deemed appropriate by the investigator. Information for all physical examinations must be included in the source document.

### Clinical Laboratory Tests

Clinical laboratory tests will be performed at Baseline before study drug administration and on Day 28 (or at Early Hospital Discharge if applicable). During the remaining days, clinical laboratory tests will be performed as per routine care as clinically indicated. The tests will include hematology with differentials, serum chemistry panel, urinalysis.

A pregnancy test will also be conducted during screening at Baseline for women of childbearing potential and on Day 28 (or at Early Hospital Discharge).

The specific laboratory test results that will be ascertained in the study are listed in Table 13. below.

TABLE 13

Clinical Laboratory Testing

| | |
|---|---|
| Laboratory Testing | Tests Included |
| Hematology | Complete blood count (CBC; red blood cells, white blood cells, platelets, hemoglobin, hematocrit) and differentials (neutrophils, lymphocytes, monocytes, eosinophils, and basophils relative and absolute). |
| Serum Chemistry | Creatinine, BUN, total bilirubin, alkaline phosphatase, AST, ALT, albumin, protein, Na, K, Cl, CO2, calcium, phosphate, glucose, CRP. |
| Urinalysis | Appearance, pH, specific gravity, protein, hemoglobin/RBCs, and leukocytes. |
| Serum or urine pregnancy test | For females of childbearing potential. |

ALT = alanine aminotransferase;
AST = aspartate aminotransferase;
BUN = blood urea nitrogen;
CBC = complete blood count;
CRP = C-reactive protein.

### Electrocardiogram

Twelve-lead ECGs will be performed per routine care only if clinically indicated. Any clinically significant worsening from Baseline will be recorded as an AE.

### Acute Kidney Injury Network (AKIN) Criteria

The AKIN criteria will be recorded at Baseline before study drug administration. For the remaining days, they will be recorded on the eCRF only if assessed per routine care at each clinical site. Any significant change should be reported as an AE.

### Troponin Levels

Troponin levels (serum or plasma per routine care at each clinical site) will be recorded at Baseline before study drug administration. For the remaining days, troponin levels will be recorded on the eCRF only if assessed per routine care at each clinical site. Any significant change should be reported as an AE.

### 5.3 Adverse Event

### Definition of Adverse Event

An AE is any untoward medical occurrence in a subject administered a pharmaceutical product and that does not necessarily have a causal relationship with this treatment. An AE can therefore be any unfavorable and unintended sign (including an abnormal laboratory finding), symptom, or disease temporally associated with the use of a study product, whether or not considered related to the study product. AEs and Serious Adverse Events (SAEs) will be collected from the time of the first dose of the study drug at Baseline until the final visit/contact; any AE or SAE occurring between obtaining informed consent and before the first dose of the study drug should be documented as part of the patient’s Medical History.

Opportunistic infections will be noted and any new infection that occurs in the study, regardless of the organism (i.e., viral or non-viral), will be captured. Additionally, the site of infection and source of culture (BAL, tracheal aspirate, sputum, blood, urine, etc.) will be recorded.

### Definition of Treatment-Emergent Adverse Events (TEAEs)

A TEAE is any condition that was not present before treatment with the study product but appeared following treatment, was present at treatment initiation but worsened during treatment, or was present at treatment initiation but resolved and then reappeared while the individual was on treatment (regardless of the intensity of the AE when the treatment was initiated).

### Definition of Serious Adverse Event

A SAE or reaction is any untoward medical occurrence that, at any dose:
Results in death
Is life-threatening*
Requires in-subject hospitalization¥ or prolongation of existing hospitalization
Results in persistent or significant disability/incapacity
Is a congenital anomaly/birth defect
Medically Significant/Medical Important Event

*Note 1: The term “life-threatening” in the definition of “serious” refers to an event in which the subject was at risk of death at the time of the event; it does not refer to an event that hypothetically might have caused death if it were more severe.

¥Note 2: As the study patients will be hospitalized at the time of screening, this criterion will be applied when a patient requires a transfer to the Intensive Care Unit or admission to another hospital department due to an adverse event.

Medical and scientific judgment should be exercised in deciding whether expedited reporting is appropriate in other situations, such as important medical events that may not be immediately life- threatening or result in death or hospitalization but may jeopardize the subject and/or may require intervention to prevent one of the other outcomes listed in the definition above. These should also usually be considered serious.

Examples of such events are intensive treatment in an emergency room or at home for allergic bronchospasm, blood dyscrasias, or convulsions that do not result in hospitalization, or development of drug dependency or drug abuse.

### Assessment of an Adverse Event

### Relationship to Study Treatment

The investigator will establish causality of the AE to the experimental treatment. The investigator must include an assessment of causality whether there is a reasonable possibility that the drug caused the event that may be sensitive to distinctions between events that may be related to the drug versus those due to the underlying disease process and/or concomitant therapies. The investigator should consider the subject’s clinical state (e.g., medical history, comorbidities, concomitant medications) and Dechallenge/Rechallenge information as results of action taken to the drug. The investigator should document the rationale for causality assessment. The following definitions will be used to determine causality of an AE:

Definitely Related: Reasonable temporal relationship to study drug administration; follows a known response pattern (i.e., drug is known to cause this AE); there is no alternative etiology.

Probably Related: Reasonable temporal relationship; follows a suspected response pattern (i.e., based on similar drugs); some evidence for a more likely alternative etiology.

Possibly Related: Reasonable temporal relationship; little or no evidence for a more likely alternative etiology.

Unlikely Related: Event with a time to drug intake that makes a relationship improbable, but not impossible, disease or other drugs provide plausible explanations.

Not Related: Does not have a temporal relationship OR Definitely due to alternative etiology.

### Adverse Event Severity

The intensity of an AE is an estimate of the relative severity of the event made by the investigator based on his or her clinical experience and familiarity with the literature. The following definitions are to be used to rate the severity of an AE:

Mild: The symptom is barely noticeable to the subject and does not influence performance of daily activities. Treatment is not ordinarily indicated.

Moderate: The symptom is sufficiently severe to make the subject uncomfortable, and performance of daily activities is influenced. Treatment may be necessary.

Severe: The symptom causes severe discomfort, and daily activities are significantly impaired or prevented. Treatment may be necessary.

### Expectedness

The medical monitor will assess the expectedness of each SAE in relation to the study product for expedited reporting.

### Time Period and Frequency for Event Assessment and Follow-Up

The occurrence of an AE or SAE may come to the attention of study personnel during study visits and interviews of a study subject presenting for medical care, or upon review by a study monitor.

All AEs, including local and systemic reactions, will be captured on the appropriate eCRF. Information to be collected includes event description, time of onset, clinician’s assessment of severity, relationship to study product (assessed only by those with the training and authority to make a diagnosis), and date of resolution/stabilization of the event. All AEs occurring while on the study must be documented appropriately regardless of relationship.

If the subject experiences an SAE at any time after the first study drug administration, the event will be recorded as an SAE in the source document and eCRF.

Before subject enrollment, study site personnel will note the occurrence and nature of each subject’s medical condition(s) in the appropriate section of the source document and eCRF. During the study, site personnel will note any change in the condition(s) and the occurrence and nature of any AE.

Any medical condition that is present at the time of consent signature will be considered as part of medical history and not reported as an AE. However, if the study subject’s condition deteriorates after the first study drug administration, it will be recorded as an AE.

If the subject experiences an AE at any time after the first study drug administration until the end of participation in the study, the event will be recorded as an AE in the source document and eCRF.

The investigator is responsible for appropriate medical care of subjects during the study. The investigator also remains responsible for following through with an appropriate health care option for all AEs that are ongoing at the end of the study. The subject should be followed until the event is resolved or stable. Follow-up frequency will be performed at the discretion of the investigator but at least once a month after hospital discharge until 60-days from treatment with the investigational product. Follow-up after discharge may be conducted via telephone.

Whenever possible, clinically significant abnormal laboratory results are to be reported using the diagnostic that resulted in the clinically significant abnormal laboratory results and not the actual abnormal test.

### Adverse Event Reporting

Investigators are responsible for monitoring the safety of subjects who are participating in this study and for alerting the sponsor of any event that seems unusual, even if this event may be considered an unanticipated benefit to the subject.

### SAE Reporting

The medical monitor will be responsible for the overall pharmacovigilance process for this study. All SAEs, related to the experimental treatment or not, occurring during the study must be reported on an SAE form to the medical monitor (see below) within 24 hours of the knowledge of the occurrence (this refers to any AE that meets one or more of the aforementioned serious criteria). The SAE reporting period ends at the end of the follow-up period or if the subject begins an alternative therapy.

The investigator must report all SAE in the eCRF (i.e., AE form) within 24 hours of their awareness of the SAE whether the SAEs are deemed drug-related or not.

If for any reason the SAE cannot be reported in the EDC, a paper SAE reporting Form must be completed and submitted to the JSS PV within 24 hours. Reporting should be done by sending the completed SAE form to the following e-mail address (faxing can also be done as a second option in case e-mailing is not possible).

The medical monitor will inform the sponsor within 1 business day of awareness of a new SAE and will evaluate all SAEs as soon as the reports are received. For each SAE received, the medical monitor will determine whether the criteria for expedited reporting to relevant regulatory authorities have been met. The medical monitor will manage the expedited reporting of relevant safety information to concerned regulatory agencies following local laws and regulations.

### 5.4 Pregnancy Reporting

Given the study inclusion/exclusion criteria which exclude pregnant or breastfeeding women and the nature of the study by which all subjects will be hospitalized it is unlikely that a female subject will become pregnant during the study. Regardless, in the unlikely scenario that a female subject or a female partner of a male subject becomes pregnant during the study or within 11 weeks after study drug administration the investigator must complete a study-specific pregnancy form upon confirmation of pregnancy and send it to the medical monitor within 24 hours of confirmation of the pregnancy to the following e-mail address (faxing can also be done as a second option in case e-mailing is not possible).

The medical monitor will report all cases of pregnancy to the sponsor in a timely manner. Post- treatment follow-up should be done to ensure the subject’s safety. Pregnancy is not itself an AE or SAE; however, maternal/fetal complications or abnormalities will be recorded as AEs or SAEs, as appropriate. The investigator will follow the pregnancy until completion or until pregnancy termination and, in the case of a live-bom offspring, to 1 month of age in that infant. The investigator will notify the medical monitor and the sponsor of the outcome as a follow up to the initial pregnancy form. All pregnancies should be reported, when applicable, to the ethics committee.

### 6. Statistical Considerations

### Sample Size Determination and Sample Size Calculations:

### Main Study

Recent results published for the evaluation of the efficacy of remdesivir in the management of COVID-19 have shown that the proportion of patients that were alive and were discharged home without oxygen or were free of respiratory failure was approximately 63%. For the Main study, we can assume that 60% of the patients treated under SOC will achieve the primary endpoint of the study.

### Stage 1 (Phase II Study)

The sample size calculations for the Phase II study are based on the following assumptions:

1:1 Randomization

Effect size: Odds Ratio = 2.00 in favor of NI-0101 for efficacy in achieving the primary endpoint with one sided alpha of 0.025 (equivalent to two-sided alpha of 0.05) and 80% power (β = 0.20).

One interim analysis at 50% of the patients reaching the 28-day follow-up.

O-Brien Fleming alpha spending function for alpha and beta.

Efficacy Bounds based on alpha – spending functions.

Non – Binding Futility Boundaries

Based on the above requirements a total of 158 evaluable patients will be required per group for a total of 316 evaluable patients. Allowing for 20% attrition a total of 396 patients will be enrolled in the study.

One blinded, comparative Interim Analysis will be conducted for the Phase II study when 50% of the patients reach the 28 – day follow up time point. The Interim Analysis will incorporate the O-Brien Fleming alpha spending function with the nominal, incremental and cumulative alpha at each analysis as described in the following Table:

| | | |
|---|---|---|
| Analysis | 1 | 2 |
| Percent | 50 | 100 |
| Sample Size (evaluable) | 158 | 316 |
| Nominal alpha | 0.002 | 0.024 |
| Incremental alpha | 0.002 | 0.023 |
| Cumulative alpha | 0.002 | 0.025 |

### Stage 2 (Phase III Study)

The sample size calculations for the Phase II study are based on the following assumptions:

1:1 Randomization

Effect size: Odds Ratio = 1.50 in favor of NI-0101 for efficacy in achieving the primary endpoint with one sided alpha of 0.025 (equivalent to two-sided alpha of 0.05) and 80% power (β = 0.20).

One interim analysis at 50% of the patients reaching the 28-day follow-up.

O-Brien Fleming alpha spending function for alpha and beta.

Efficacy Bounds based on alpha – spending functions.

Non – Binding Futility Boundaries.

Based on the above requirements a total of 884 evaluable patients will be required for Stage 2 (Phase III study). Of these, 442 will be treated with NI-0101 + SOC and 442 will be treated with

Placebo + SOC. Allowing for 20% attrition, a total of 1,106 patients will be enrolled in this Stage.

One blinded, comparative interim analysis has been planned during Stage 2 (Phase III). The following table describes the nominal, incremental and cumulative alpha at each analysis based on the O-Brien-Fleming alpha spending function.

| | | |
|---|---|---|
| Analysis | 1 | 2 |
| Percent | 50 | 100 |
| Sample Size (evaluable) | 442 | 884 |
| Nominal alpha | 0.002 | 0.024 |
| Incremental alpha | 0.002 | 0.023 |
| Cumulative alpha | 0.002 | 0.025 |

### Sub (Exploratory) Study

The following assumptions have been used to determine the sample size requirements for the Sub (Exploratory) Study:

Primary Efficacy Outcome Measure = Cumulative Mortality at 60 days.

Estimated Cumulative Mortality for the SOC group =40%.

Minimally clinically important relative reduction in mortality =25% -50%. Equivalent to 10%-20% absolute reduction in mortality.

For the purposes of this sub-study a 50% relative reduction (20% absolute) reduction will be used as the estimated effect size.

Estimated cumulative mortality rate in SOC+NI-0101 =20%.

Hazard Rate Ratio for SOC/NI-0101+SOC = 2.2892.

Significance (one – tailed) = 0.10 (α = 0.10); This level of significance has been used given the exploratory nature of this sub-study.

Power = 80% (β = 0.20).

Two interim and one final analyses (total = 3 looks) with Obrien-Fleming alpha spending functions and non – binding futility boundaries.

Three analyses in total.

Total number of events (deaths) = 30.

Estimated number of patients = 100.

Based on the above requirements, enrolment for the sub-study will be completed when a total of 30 events (deaths) have been observed. Based on the currently available estimates it is anticipated that approximately 100 patients will be enrolled in the Sub (Exploratory) Study.

### 6.2 Analysis Populations

The Primary Efficacy Analysis will be conducted on the intent-to-treat (ITT) population. A supportive analysis for the primary endpoint will also be conducted on the per-protocol (PP) population. Safety will be evaluated on the safety (SAF) population.

Intent-to-Treat Population (ITT): This population will include all enrolled subjects who are randomized. All subjects will be analyzed according to the treatment group to which they were randomized.

The Per Protocol Population (PP): The PP population will include all enrolled subjects who were randomized and with at least one dose of the study product, with no significant protocol deviations, and who provided evaluable data for the primary endpoint. All subjects will be analyzed according to the treatment group they received.

The Safety Population (SAF): This population is defined as all subjects who received at least one dose of the study product. All subjects will be analyzed according to the treatment group they received.

### 6.3 Statistical Analyses

### 6.3.1 General Considerations

Descriptive statistics including the number of subjects, mean, median, standard deviation, minimum, maximum and 95% CI of the mean for continuous parameters and frequency distributions (number and proportion) for categorical parameters will be reported.

To verify the comparability of the two treatment groups, key baseline patient and disease characteristics will be compared with the independent-samples t-test (or the Mann-Whitney test if the normality assumption is violated) for continuous variables and the Chi-Square test for categorical variables. Baseline characteristics for which clinically important differences, defined as a two-fold factor, or statistically noteworthy defined as P < 0.15, will be considered as potential confounders and will be included in the sensitivity analyses described later.

There will be no imputations for missing data. All analyses will be conducted on observed data.

All details regarding the efficacy and safety variable definitions, analyses strategy, statistical justification, and techniques for handling missing values will be detailed in a separate Statistical Analysis Plan (SAP) that will be finalized before the first planned interim analysis. All statistical tests will be two-sided and will be performed with a significance level of 0.05 unless otherwise specified in the SAP. The analysis will be performed using SAS® Version 9 or higher.

### 6.3.2. Analyses to Address Study Objectives:

### 6.3.2.1. Primary Efficacy Endpoint

The between-group difference in the primary efficacy endpoint will be assessed with simple logistic regression with the treatment group being the primary independent variable and achieving the primary efficacy endpoint will be the binary dependent variable. The Odds Ratio estimated from the Logistic Regression will be used as the estimate of the Relative Risk (OR) for reaching the primary efficacy endpoint. The 95% Confidence Intervals around the point estimate of the OR will be used to assess the precision of the estimate, while statistical significance will be assessed with the Wald Chi-Square and Goodness of Fit will be assessed with the Hosmer Lemeshow Test.

The following sensitivity analyses will be conducted:

Multivariate logistic regression analysis with site, use of antivirals, and baseline COVID- 19 disease severity entered as covariates. More specifically, in this analysis the study site will be entered as a categorical variable, antiviral use will be entered as a binary variable, baseline COVID-19 severity will be entered as an ordinal variable with Level 4 used as the reference. Sites that enroll less than 10 patients will be aggregated in one category. A second confirmatory analysis will be conducted in which the logistic regression model will also include the site x treatment group and baseline COVID-19 severity x treatment group interaction terms.

Stratification by baseline COVID-19 disease severity.

A multivariate logistic regression model with all potential confounders, identified as described above, entered as covariates.

Multivariate logistic regression in which all the interaction terms of treatment group with potential confounders.

Site effect will be assessed by the following:

Descriptive statistics of the treatment effect by site.

Including the site as a random effect in the logistic regression model used for the primary efficacy analysis.

Including the site × treatment group interaction term in the logistic regression model used for the primary efficacy analysis.

Sensitivity analyses will include stratification by baselines disease severity and multivariate logistic regression analyses with patient demographics, relevant comorbidities and concomitant medication use will be entered as covariates.

### 6.3.2.2. Secondary Efficacy Endpoints

The following table summarizes the analytical methods that will be used to assess between treatment group differences with the study secondary endpoints:

| | | |
|---|---|---|
| Endpoint | Methods: | Population |
| Time to Therapeutic response (Primary Efficacy Endpoint) | Survival Analysis using Kaplan Meier Estimator and Breslow-Day log-rank test. | All |
| | Cox’s proportional hazards for multivariate analyses. | |
| The proportion of patients with clinical improvement, defined as a decrease of two points or more on the WHO 9–point ordinal scale at Day 28. | Logistic Regression to estimate Odds Ratio with 95% CI to assess precision and Wald-Chi square to assess statistical significance. | All |
| | Multivariate Logistic Regression for Multi-variate analysis. | |
| The proportion of patients with clinical improvement, defined as a decrease of two points or more on the WHO 9–point ordinal scale at Day 60. | Logistic Regression to estimate Odds Ratio with 95% CI to assess precision and Wald-Chi square to assess statistical significance. | All |
| | Multivariate Logistic Regression for Multi-variate analysis. | |
| The proportion of patients with clinical improvement, defined as a decrease of one point or more on the WHO 9–point ordinal scale at Day 28. | Logistic Regression to estimate Odds Ratio with 95% CI to assess precision and Wald-Chi square to assess statistical significance. | |
| | Multivariate Logistic Regression for Multi-variate analysis. | |
| The proportion of patients with clinical improvement, defined as a decrease of one point or more on the WHO 9–point ordinal scale at Day 60. | Logistic Regression to estimate Odds Ratio with 95% CI to assess precision and Wald-Chi square to assess statistical significance. | |
| | Multivariate Logistic Regression for Multi-variate analysis. | |
| The proportion of patients that are alive and discharged home without any need for oxygen support (WHO Scale of ≤ 2) at Day 28. | Logistic Regression to estimate Odds Ratio with 95% CI to assess precision and Wald-Chi square to assess statistical significance. | All |
| | Multivariate Logistic Regression for Multi-variate analysis. | |
| The proportion of patients that are alive and free of respiratory failure (WHO scale ≤ 4) at Day 28. | Logistic Regression to estimate Odds Ratio with 95% CI to assess precision and Wald-Chi square to assess statistical significance. | All |
| | Multivariate Logistic Regression for Multi-variate analysis. | |
| The proportion of patients that are alive and discharged home without any need for oxygen support (WHO Scale of ≤ 2) at Day 60. | Logistic Regression to estimate Odds Ratio with 95% CI to assess precision and Wald-Chi square to assess statistical significance. | All |
| | Multivariate Logistic Regression for Multi-variate analysis. | |
| The proportion of patients that are alive and free of respiratory failure (WHO scale ≤ 4) at Day 60. | Logistic Regression to estimate Odds Ratio with 95% CI to assess precision and Wald-Chi square to assess statistical significance. | All |
| | Multivariate Logistic Regression for Multi-variate analysis. | |
| Time to clinical improvement by 2 points on the nine-point ordinal scale described above. | Survival Analysis using Kaplan Meier Estimator and Breslow-Day log-rank test. | All |
| | Cox’s proportional hazards for multivariate analyses. | |
| Time to clinical improvement by 1 point on the nine-point ordinal scale described above. | Survival Analysis using Kaplan Meier Estimator and Breslow-Day log-rank test. | All |
| | Cox’s proportional hazards for multivariate analyses. | |
| Change in the NEWS-2 Scale at 28 days. | Student’s t-test. | All |
| | Generalized Linear Models with Repeated Measures. | |
| Time to NEWS-2 = 0 | Survival Analysis using Kaplan Meier Estimator and Breslow-Day log-rank test. | All |
| | Cox’s proportional hazards for multivariate analyses. | |
| The proportion of patients that experience disease progression, defined as an increase of one point or more in the WHO 9-point ordinal scale at Day 28. | Logistic Regression to estimate Odds Ratio with 95% CI to assess precision and Wald-Chi square to assess statistical significance. | All |
| | Multivariate Logistic Regression for Multi-variate analysis. | |
| Ventilator-free days | Survival Analysis using Kaplan Meier Estimator and Breslow-Day log-rank test. | All |
| | Cox’s proportional hazards for multivariate analyses. | |
| Duration of ventilation | Survival Analysis using Kaplan Meier Estimator and Breslow-Day log-rank test. | All |
| | Cox’s proportional hazards for multivariate analyses. | |
| Mortality Rate (Primary Endpoint for the Sub- Exploratory Study). | Logistic Regression to estimate Odds Ratio with 95% CI to assess precision and Wald-Chi square to assess statistical significance. | All |
| | Multivariate Logistic Regression for Multi-variate analysis. | |
| Duration of hospitalization | Survival Analysis using Kaplan Meier Estimator and Breslow-Day log-rank test. | All |
| | Cox’s proportional hazards for multivariate analyses. | |
| Time to independence from supplementary oxygen therapy | Survival Analysis using Kaplan Meier Estimator and Breslow-Day log-rank test. | Levels 4-7 of the COVID-19 scale at Baseline |
| | Cox’s proportional hazards for multivariate analyses. | |
| Time to normalization of oxygen saturation, defined as SaO2/SpO2) > 94% sustained a minimum of 24 hours | Survival Analysis using Kaplan Meier Estimator and Breslow-Day log-rank test. | Patients with SaO2/SpO2 < 94% at Baseline |
| | Cox’s proportional hazards for multivariate analyses. | |
| Change in Sequential Organ Failure Assessment (SOFA) score, daily while hospitalized | Student’s t-test. | All |
| | Generalized Linear Models with Repeated Measures. | |
| Radiological response based on Thoracic Computerized Tomography Scan (CT-Scan) or Chest X-Ray | Descriptive statistics with the proportion of patients showing: | All |
| | Improvement | |
| | No Change | |
| | Deterioration | |
| Change in cytokines, IL-6, and C-reactive protein (CRP) levels | Student’s t-test. | All |
| | Generalized Linear Models with Repeated Measures. | |
| Time to resolution of fever for at least 48 hours without antipyretics Defined as post-baseline body temperature <37.2° C. (oral), or <37.6° C. (rectal or tympanic) or <36.8° C. (temporal or axillary) | Survival Analysis using Kaplan Meier Estimator and Breslow-Day log-rank test. | Patient with temperature ≥37.2° C. (oral), or <37.6° C. (rectal or tympanic) or ≥36.8° C. (temporal or axillary |
| | Cox’s proportional hazards for multivariate analyses. | |
| The decision by the attending physician to initiate treatment with another targeted immunomodulator (e.g., dexamethazone) | Logistic Regression to estimate Odds Ratio with 95% CI to assess precision and Wald-Chi square to assess statistical significance. | All |
| | Multivariate Logistic Regression for Multi-variate analysis. | |
| Change in Berlin ARDS severity | Median test | Patients with ARDS at Baseline |
| | Kruskal-Wallis test | |
| Change in Acute Kidney Injury Network (AKIN) classification | Median test | All |
| | Kruskal-Wallis test | |
| Change in troponin levels | Student’s t-test. | All |
| | Generalized Linear Models with Repeated Measures. | |
| Duration of ECMO (Sub-Study Only) | Survival Analysis using Kaplan Meier Estimator and Breslow-Day log-rank test. | Level 7 |
| | Cox’s proportional hazards for multivariate analyses. | |

### 6.3.2.3. Safety Analysis

TEAEs and serious TEAEs will be presented and tabulated according to MedDRA classification. Descriptions of AEs will include the seriousness, severity, relationship to study product, and outcome.

Reported TEAEs and serious TEAEs will be summarized by the number and proportion of subjects reporting the events, as well as by System Organ Class and Preferred Term within each SOC. In addition to the overall TEAEs and serious TEAEs summaries by seriousness, severity, and relationship to study product will also be produced. For the summary of AEs by severity, each subject will be counted only once within a SOC or PT within each SOC by using the AEs with the highest intensity within each category for each analysis. For the summary of AEs by relationship to study product, each subject will be counted only once within a SOC or PT within each SOC by using the AEs with the greatest reported relationship within each category.

All information about TEAEs and serious TEAEs noted during the study will be listed by subject, detailing verbatim, SOC, PT, start date, stop date, seriousness, severity, outcome, and relationship to study product. The AE onset will also be shown relative (in number of days) to the day of study drug administration.

Results from vital signs, ECGs, physical examination, and clinical laboratory tests, will be tabulated by treatment and visit using descriptive statistics. The value at each visit as well as the change from baseline will be presented descriptively.

No inferential statistics will be done on safety variables.

The published results of the remdesivir study show that 26% of the patients in the SOC group experienced a Serious Adverse Events and that 13% experienced a Grade 3-4 Serious Adverse Event. Assuming that in the current study 25% of the patients treated with SOC will experience a Serious Adverse Event. The overall study will have 80% power to detect an Odds Ratio of 2.00

indicating increased risk for Serious Adverse Events for the NI-0101 group. The Independent DMC will review the incidence of treatment-emergent Serious Adverse Events in the two treatment groups and will determine whether a clinically important increase (Odds Ratio > 1.50) in the risk of a Serious Adverse Event is observed.

### Other Analyses

Concomitant medications will be coded with the World Health Organization-Drug Global Dictionary (WHO Drug Global) and summarized by the number and proportion of subjects using each medication.

Also, a list of subjects who discontinued from the study along with the associated reason will be provided.

For all subjects, descriptive summaries of baseline characteristics, including demographic data and medical/surgical history, and of subject disposition will be presented.

Protocol deviations will be summarized by treatment and category.

### Subgroup Analysis

Subgroup analyses will be conducted for the primary endpoint by:
Baseline COVID-19 severity level;
Age groups;
Gender;
Location of residence (home or long-term care facility);
Suspected route of transmission (travel, contact with an individual that travelled to high-risk regions, community, unknown);
Presence of relevant comorbid conditions;
Prior vaccination history;
Prior influenza disease.

### Planned Interim Analyses

During the Phase II study, the Interim Analysis will be conducted when data are available for the primary endpoint assessment from 50% of the patients. The Interim Analysis will be blinded with respect to treatment allocation. More specifically, the treatment groups will be identified as Group A or Group B. The results of the Interim Analysis will be reported to the Independent Data Monitoring Committee (DMC). The results of the Interim Analysis will be used to determine whether the study should be terminated for futility or safety.

The following table describes the Interim Analysis parameters and the requirements for early termination due to futility.

| Parameter | Looks | |
|---|---|---|
| | 1 | 2 |
| Sample Size | 158 | 316 |
| Lower Efficacy Bound¹ | -8.000 | -8.000 |
| Upper Efficacy Bound¹ | 2.963 | 1.969 |
| Futility Bound | 0.558 | 1.969 |
| Nominal Alpha | 0.002 | 0.024 |
| Incremental Alpha | 0.002 | 0.025 |
| Cumulative Alpha | 0.002 | 0.025 |
| Exit Probability Under H₁ | 24.691 | 75.309 |
| Cumulative Exit probability Under H₁ | 24.691 | 100.00 |
| Nominal beta | 0.288 | 0.024 |
| Incremental beta | 0.070 | 0.130 |
| Cumulative beta | 0.070 | 0.200 |
| Exit Probability Under H₀ | 71.317 | 28.683 |
| Cumulative Exit probability Under H₀ | 71.317 | 100.00 |

1 Efficacy bounds are presented for reference only. The study will not be terminated due to efficacy.

During the Phase III study there will be one blinded, comparative interim analysis, the results of which will be used to determine if the study should continue or whether early termination for futility is warranted. The following table describes the parameters and futility termination conditions for the Phase III Interim Analysis.

| Parameter | Looks | |
|---|---|---|
| | 1 | 2 |
| Sample Size | 442 | 884 |
| Lower Efficacy Bound¹ | -8.000 | -8.000 |
| Upper Efficacy Bound¹ | 2.963 | 1.969 |
| Futility Bound | 0.558 | 1.969 |
| Nominal Alpha | 0.002 | 0.024 |
| Incremental Alpha | 0.002 | 0.023 |
| Cumulative Alpha | 0.002 | 0.025 |
| Exit Probability Under H₁ | 24.689 | 75.311 |
| Cumulative Exit probability Under H₁ | 24.691 | 100.00 |
| Nominal beta | 0.289 | 0.024 |
| Incremental beta | 0.069 | 0.130 |
| Cumulative beta | 0.069 | 0.199 |
| Exit Probability Under H₀ | 71.274 | 28.726 |
| Cumulative Exit probability Under H₀ | 71.274 | 100.00 |

The following table describes the Interim Analyses parameters for the Sub (Exploratory) Study.

| Parameter | Looks | | |
|---|---|---|---|
| | 1 | 2 | 3 |
| Sample Size | 10 | 20 | 30 |
| Lower Efficacy Bound¹ | -8.000 | -8.000 | -8.000 |
| Upper Efficacy Bound¹ | 2.261 | 1.722 | 1.359 |
| Futility Bound | -0.586 | 0.638 | 1.359 |
| Nominal Alpha | 0.004 | 0.043 | 0.087 |
| Incremental Alpha | 0.004 | 0.040 | 0.056 |
| Cumulative Alpha | 0.004 | 0.044 | 0.100 |
| Exit Probability Under H₁ | 12.528 | 54.899 | 32.843 |
| Cumulative Exit probability Under H₁ | 12.528 | 67.157 | 100.00 |
| Nominal beta | 0.721 | 0.262 | 0.087 |
| Incremental beta | 0.030 | 0.095 | 0.085 |
| Cumulative beta | 0.030 | 0.125 | 0.210 |
| Exit Probability Under H₀ | 28.340 | 50.609 | 21.050 |
| Cumulative Exit probability Under H₀ | 28.340 | 78.950 | 100.00 |

1 Efficacy bounds are presented for reference only. The study will not be terminated due to efficacy.

### Example 3 – Use of NI-0801 Anti-IP10 Antibody for Treatment of Acute Respiratory Distress Syndrome – Phase 2 Study Design

Phase 2 study - multicenter, two-arm (NI-0801+SOC vs SOC), double-blind. Sequential adaptive design with the ability to declare efficacy at interim analysis. Stage 1 of the study will assess whether there is an efficacy signal on multiple putative primary efficacy outcomes (see Primary Endpoints below). 28-day study period.

Dosing - Single IV infusion of 15 mg/kg (may include option for multiple subsequent doses if patient is not improving).

Primary Endpoint(s) - Proportion of patients that decline defined as either admission to an intensive care unit (ICU), the use of mechanical ventilation, or death. Table 14 shows recent epidiomological data related to ARDS.

TABLE 14

Epidemiological Data Related to Acute Respiratory Distress Syndrome (ARDS)

| Source | N | ARDS | ICU | Death | Ventilation (Invasive) | Composite |
|---|---|---|---|---|---|---|
| Huang, Lancet, 2020 | 41 | 29% | 32% | 15% | 10% | N/A |
| Chen, Lancet, 2020 | 99 | 17% | 23% | 11% | 4% | N/A |
| Guan, NEJM, 2020 | 173 | 16% | 19% | 8% | 15% | 25% |
| Wang, JAMA, 2020 | 138 | 20% | 26% | 4% | 11% | N/A |

The time from randomization to an improvement of two points (from the status at randomization) is measured on a seven-category ordinal scale or live discharge from the hospital, whichever comes first (The seven-category ordinal scale consists of the following categories: 1, not hospitalized with resumption of normal activities; 2, not hospitalized, but unable to resume normal activities; 3, hospitalized, not requiring supplemental oxygen; 4, hospitalized, requiring supplemental oxygen; 5, hospitalized, requiring nasal high-flow oxygen therapy, noninvasive mechanical ventilation, or both; 6, hospitalized, requiring ECMO, invasive mechanical ventilation, or both; and 7, death). See powering below (from Wang et al. NEJM, 2020).

The duration from start of treatment to normalization of pyrexia, respiratory rate and SPO2 and relief of cough (where there are relevant abnormal symptoms at enrolment) that is maintained for at least 72 h.

Secondary Endpoints – 1) Proportion of patients admitted to ICU admission; 2) Proportion of patients who require ventilation; 3) Proportion of patients who progress to ARDS; 4) Proportion of patients who die; 5) Duration of hospitalization; 6) Time to independence from non-invasive mechanical ventilation calculated in days; 7) Time to independence from oxygen therapy in days; 8) Change of the SOFA (Sequential Organ Failure Assessment): It evaluates 6 variables, each representing an organ system (one for the respiratory, cardiovascular, hepatic, coagulation, renal and neurological systems), and scored from 0 (normal) to 4 (high degree of dysfunction/failure). Thus, the maximum score may range from 0 to 24; 9) Radiological response: Thoracic CT scan or Chest XR

Inclusion Criteria – 1) Any gender; 2) No age limit; 3) Informed consent for participation in the study; 4) Virological diagnosis of Sars-CoV2 infection (PCR); 5) Hospitalized due to clinical/instrumental diagnosis of pneumonia; 6) Blood oxygen saturation at rest in ambient air ≤93% or 200<PaO2/FiO2≤300.

Exclusion Criteria – 1) Known hypersensitivity to NI-0101 or its excipients; 2) Patient has already progressed to ARDS; 3) Patient being treated with immunomodulators or anti-rejection drugs; 4) Known other active infections or other clinical condition that contraindicate NI-0101 and cannot be treated or solved according to the judgement of the clinician; 5) Possibility of the subject being transferred to a non-study hospital within 72 h; 6) Women of childbearing potential who are lactating or pregnant as determined by urine pregnancy test at Screening.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating, preventing or alleviating a symptom of acute respiratory distress syndrome (ARDS) in a subject in need thereof comprising administering to the subject a composition comprising an antibody that binds specifically to a Toll-like Receptor 4 (TLR4) and MD-2 complex.

2. The method of claim 1, wherein the antibody comprises:
a) a heavy chain variable region comprising a complementarity determining region 1 (CDRH1) comprising the amino acid sequence of SEQ ID NOs: 1, 20 or 28;
a complementarity determining region 2 (CDRH2) comprising the amino acid sequence of SEQ ID NOs: 2, 21 or 29; and
a complementarity determining region 3 (CDRH3) comprising the amino acid sequence of SEQ ID NOs: 3, 22, 30, 186 or 187; and
b) a light chain variable region comprising a complementarity determining region 1 (CDRL1) comprising the amino acid sequence of SEQ ID NOs: 4, 24, 33;
a complementarity determining region 2 (CDRL2) comprising the amino acid sequence of SEQ ID NOs: 5, 25 or 34; and
a complementarity determining region 3 (CDRL3) comprising the amino acid sequence of SEQ ID NOs: 6, 17, 26, 35, 188, 189, 190 or 191.

3. The method of claim 2, wherein the antibody comprises a CDRH1 region comprising the amino acid sequence of SEQ ID NO: 1; a CDRH2 region comprising the amino acid sequence of SEQ ID NO: 2, a CDRH3 region comprising the amino acid sequence of SEQ ID NO: 3; a CDRL1 region comprising the amino acid sequence of SEQ ID NO: 4; a CDRL2 region comprising the amino acid sequence of SEQ ID NO: 5; and a CDRL3 region comprising an amino acid sequence of SEQ ID NO: 6.

4. The method of claim 2, wherein the antibody comprises a CDRH1 region comprising the amino acid sequence of SEQ ID NO: 1; a CDRH2 region comprising the amino acid sequence of SEQ ID NO: 2, a CDRH3 region comprising the amino acid sequence of SEQ ID NO: 3; a CDRL1 region comprising the amino acid sequence of SEQ ID NO: 4; a CDRL2 region comprising the amino acid sequence of SEQ ID NO: 5; and a CDRL3 region comprising an amino acid sequence of SEQ ID NO: 17.

5. The method of claim 2, wherein the antibody comprises a CDRH1 region comprising the amino acid sequence of SEQ ID NO: 20; a CDRH2 region comprising the amino acid sequence of SEQ ID NO: 21, a CDRH3 region comprising the amino acid sequence of SEQ ID NO: 22; a CDRL1 region comprising the amino acid sequence of SEQ ID NO: 24; a CDRL2 region comprising the amino acid sequence of SEQ ID NO: 25; and a CDRL3 region comprising an amino acid sequence of SEQ ID NO: 26.

6. The method of claim 2, wherein the antibody comprises a CDRH1 region comprising the amino acid sequence of SEQ ID NO: 28; a CDRH2 region comprising the amino acid sequence of SEQ ID NO: 29, a CDRH3 region comprising the amino acid sequence of SEQ ID NO: 30; a CDRL1 region comprising the amino acid sequence of SEQ ID NO: 33; a CDRL2 region comprising the amino acid sequence of SEQ ID NO: 34; and a CDRL3 region comprising an amino acid sequence of SEQ ID NO: 35.

7. The method of claim 1, wherein the antibody comprises a heavy chain variable region comprising the amino acid sequence of SEQ ID NOs: 350, 14, 15, 19, 27, 31, 36 or 38 and a light chain variable region comprising the amino acid sequence of SEQ ID NOs: 351, 16, 18, 23, 32, 40, 42, 44 or 46.

8. The method of claim 7, wherein said antibody comprises a heavy chain variable region comprising the amino acid sequence of SEQ ID NO: 350 and a light chain variable region comprising the amino acid sequence of SEQ ID NO: 351.

9. The method of claim 7, wherein said antibody comprises a heavy chain variable region comprising the amino acid sequence of SEQ ID NO: 14 and a light chain variable region comprising the amino acid sequence of SEQ ID NO: 16.

10. The method of claim 7, wherein said antibody comprises a heavy chain variable region comprising the amino acid sequence of SEQ ID NO: 14 and a light chain variable region comprising the amino acid sequence of SEQ ID NO: 18.

11. The method of claim 7, wherein said antibody comprises a heavy chain variable region comprising the amino acid sequence of SEQ ID NO: 15 and a light chain variable region comprising the amino acid sequence of SEQ ID NO: 16.

12. The method of claim 7, wherein said antibody comprises a heavy chain variable region comprising the amino acid sequence of SEQ ID NO: 15 and a light chain variable region comprising the amino acid sequence of SEQ ID NO: 18.

13. The method of claim 7, wherein said antibody comprises a heavy chain variable region comprising the amino acid sequence of SEQ ID NO: 19 and a light chain variable region comprising the amino acid sequence of SEQ ID NO: 23.

14. The method of claim 7, wherein said antibody comprises a heavy chain variable region comprising the amino acid sequence of SEQ ID NO: 27 and a light chain variable region comprising the amino acid sequence of SEQ ID NO: 32.

15. The method of claim 7, wherein said antibody comprises a heavy chain variable region comprising the amino acid sequence of SEQ ID NO: 31 and a light chain variable region comprising the amino acid sequence of SEQ ID NO: 32.

16. The method of claim 7, wherein said antibody comprises a heavy chain variable region comprising the amino acid sequence of SEQ ID NO: 36 and a light chain variable region comprising the amino acid sequence of SEQ ID NO: 40.

17. The method of claim 7, wherein said antibody comprises a heavy chain variable region comprising the amino acid sequence of SEQ ID NO: 36 and a light chain variable region comprising the amino acid sequence of SEQ ID NO: 42.

18. The method of claim 7, wherein said antibody comprises a heavy chain variable region comprising the amino acid sequence of SEQ ID NO: 36 and a light chain variable region comprising the amino acid sequence of SEQ ID NO: 44.

19. The method of claim 7, wherein said antibody comprises a heavy chain variable region comprising the amino acid sequence of SEQ ID NO: 36 and a light chain variable region comprising the amino acid sequence of SEQ ID NO: 46.

20. The method of claim 7, wherein said antibody comprises a heavy chain variable region comprising the amino acid sequence of SEQ ID NO: 38 and a light chain variable region comprising the amino acid sequence of SEQ ID NO: 40.

21. The method of claim 7, wherein said antibody comprises a heavy chain variable region comprising the amino acid sequence of SEQ ID NO: 38 and a light chain variable region comprising the amino acid sequence of SEQ ID NO: 42.

22. The method of claim 7, wherein said antibody comprises a heavy chain variable region comprising the amino acid sequence of SEQ ID NO: 38 and a light chain variable region comprising the amino acid sequence of SEQ ID NO: 44.

23. The method of claim 7, wherein said antibody comprises a heavy chain variable region comprising the amino acid sequence of SEQ ID NO: 38 and a light chain variable region comprising the amino acid sequence of SEQ ID NO: 46.

24. The method of any one of the preceding claims, wherein the antibody is a monoclonal antibody.

25. The method of any one of the preceding claims, wherein the antibody is a humanized antibody.

26. The method of any one of the preceding claims, wherein the antibody is an IgG isotype.

27. The method of claim 26, wherein said antibody is an IgG1 isotype.

28. The method of claim 27, wherein amino acid residues at EU positions 325-328 of the CH2 domain of a IgG1 consist of an amino acid motif of SKAF (SEQ ID NO: 193).

29. The method of any one of the preceding claims, wherein the antibody is administered by inhalation, nasally, intravenously, subcutaneously, intramuscularly or any combination thereof.

30. The method of claim 29, wherein the antibody is administered intravenously.

31. The method of any one of the preceding claims, wherein the antibody is administered at a dose of about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, or about 25 mg/kg.

32. The method of any one of the preceding claims, wherein the antibody is administered at least one time, at least 2 times, at least 3 times, at least 4 times, at least 5 times, at least 6 times, at least 7 times, at least 8 times, at least 9 times or at least 10 times.

33. The method of any one of the preceding claims, wherein the antibody is administered one time, at a dose of about 15 mg/kg, intravenously.

34. The method of any one of the preceding claims, wherein a symptom of ARDS is acute onset of bilateral alveolar infiltrates, hypoxemia, acute hypoxemia, lobar collapse, lung collapse, productive cough, fatigue, fever, chest pain, shortness of breath, labored breathing, increased heart rage, low blood pressure, confusion, extreme tiredness, respiratory failure, pulmonary vascular leak, pulmonary edema, alveolar epithelial cell injury, alveolar endothelial cell injury, alveolar capillary membrane barrier disruption or any combination thereof.

35. The method of any one of the preceding claims, wherein the subject has a coronavirus infection, a viral infection, an influenza infection, sepsis, an aspiration pneumonitis, an infectious pneumonia, severe trauma, a fracture, a pulmonary contusion, an inhalation injury, a transfusion related injury, HSCT, pancreatitis, cytokine storm from cancer therapeutics, a collagen vascular disease, a drug effect from ingestants, a drug effect from inhalants, shock, acute eosinophilic pneumonia, immunologically mediated pulmonary hemorrhage and vasculitis, radiation pneumonitis or any combination thereof.

36. The method of any one of claims 1-34, wherein the subject has a coronavirus infection.

37. The method of any one of claims 1-34, wherein the subject is suspected of having a coronavirus infection.

38. The method of any one of claims 1-34, wherein the subject has been exposed to a coronavirus or wherein the subject is suspected to have been exposed to a coronavirus; and the subject has not developed a symptom of a coronavirus infection.

39. The method of any one of claims 36-38, wherein the coronavirus is 229E alpha coronavirus, NL63 alpha coronavirus, OC43 beta coronavirus, HKU1 beta coronavirus, Middle East Respiratory Syndrome beta coronavirus (MERS-CoV or MERS), Severe Acute Respiratory Syndrome beta corona virus (SARS-CoV or SARS), a B1.1.7 variant of SARS-CoV, a B.1.351 variant of SARS-CoV, a P.1 variant of SARS-CoV, a B.1.427 variant of SARS-CoV, a B.1.4 variant of SARS-CoV, or novel coronavirus that causes coronavirus disease 2019 (SARS-CoV-2 or COVID-19).

40. The method of any one of claims 36-39, wherein a symptom of the coronavirus infection is shortness of breath, dyspnea, dry cough, fever, runny nose, nasal congestion, anosmia, loss of smell, muscle aches, muscle pains, fatigue, respiratory sputum production, headache, vomiting, hemoptysis, sore throat, myalgia, diarrhea or any combination thereof.

41. The method of claim any one of the preceding claims, further comprising administering an antiviral drug, an ACE inhibitor, or immune booster drug, a corticosteroid or any combination thereof.

42. The method of claim 41, wherein the antiviral drug is Remdesivir, bamlanivimab, etesevimab, casirivimab, imdevimab or a monoclonal antibody targeting the virus.

43. The method of claim 41, wherein the ACE inhibitor is hydroxychloroquine or a soluble recombinant ACE2.

44. The method of claim 41, wherein the immune booster drug is an anti-IL6 antibody, an anti-IP-10 antibody, an anti IL-1 antibody or an anti-TNF antibody.

45. The method of claim 44, wherein the IL6 antibody is tocilizumab or sarilumab.

46. The method of claim 41, wherein the immune booster drug is atorvastatin or pravastatin.

47. The method of claim 41, wherein the corticosteroid is dexamethasone.

48. An injectable pharmaceutical formulation comprising about 10 mg/mL of an antibody that binds specifically to TLR4, about 1.88 mg/mL of L-Histidine, about 2.70 mg/mL of L-Histidine monohydrochloride, monohydrate, about 68.46 mg/mL of sucrose and about 0.05 mg/mL of polysorbate 80.

49. The injectable pharmaceutical formulation of claim 48, wherein the antibody comprises a CDRH1 region comprising the amino acid sequence of SEQ ID NO: 1; a CDRH2 region comprising the amino acid sequence of SEQ ID NO: 2, a CDRH3 region comprising the amino acid sequence of SEQ ID NO: 3; a CDRL1 region comprising the amino acid sequence of SEQ ID NO: 4; a CDRL2 region comprising the amino acid sequence of SEQ ID NO: 5; and a CDRL3 region comprising an amino acid sequence of SEQ ID NO: 6.

50. The injectable pharmaceutical formulation of claim 48, wherein the antibody comprises a heavy chain variable region comprising the amino acid sequence of SEQ ID NO: 350 and a light chain variable region comprising the amino acid sequence of SEQ ID NO: 351.

51. An injectable pharmaceutical formulation comprising about 150 mg/mL of an antibody that binds specifically to TLR4, about 5.24 mg/mL of L-Histidine monohydrochloride, about 40.15 mg/mL of L-Arginine monohydrochloride, about 1.65 mg/mL of L-Arginine and about 0.20 mg/mL of polysorbate 80.

52. The injectable pharmaceutical formulation of claim 51, wherein the antibody comprises a CDRH1 region comprising the amino acid sequence of SEQ ID NO: 1; a CDRH2 region comprising the amino acid sequence of SEQ ID NO: 2, a CDRH3 region comprising the amino acid sequence of SEQ ID NO: 3; a CDRL1 region comprising the amino acid sequence of SEQ ID NO: 4; a CDRL2 region comprising the amino acid sequence of SEQ ID NO: 5; and a CDRL3 region comprising an amino acid sequence of SEQ ID NO: 6.

53. The injectable pharmaceutical formulation of claim 51, wherein the antibody comprises a heavy chain variable region comprising the amino acid sequence of SEQ ID NO: 350 and a light chain variable region comprising the amino acid sequence of SEQ ID NO: 351.
